(19) United States
(12) Patent Application Publication
(10) Pub. No.: US 2008/0269596 A1
(43) Pub. Date: Oct. 30, 2008

(54) Orthpaedic Monitoring Systems, Methods, Implants and Instruments

(76) Inventors: Ian Revie, North Yorkshire (GB); Alan Ashby, North Yorkshire (GB); Thorsten Burger, Muenchen (DE); Stefan Vilsmeier, Kufstein (AT); Assaf Govari, Haifa (IL); Dudi Reznick, Shimshit (IL); Pesach Susel, Haifa (IL); Avi Shalgi, Tel Aviv (IL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/598,626

(22) PCT Filed: Mar. 10, 2005
(86) PCT No.: PCT/GB05/00933

§ 371 Date: Oct. 23, 2007

Related U.S. Application Data
(60) Provisional application No. 60/575,389, filed on Jun. 1, 2004.

(30) Foreign Application Priority Data
Mar. 10, 2004 (EP) 04251371.3

(51) Int. Cl. A61B 5/00; G06Q 50/00; A61B 19/00

(52) U.S. Cl. 600/424; 606/130; 705/28

(57) ABSTRACT

Orthopaedic operating systems, operating rooms, computer aided surgical methods, trackable implants, instruments and surgical planning and IGS software applications are described. The integrated surgical system is for use in an orthopaedic operating room to enable a surgeon to carry out a computer aided surgical procedure on a subject. A subject support, wireless magnetic tracking system, registration system, at least a first display device, a control system and a surgeon interface operable by the surgeon to control operation of the plurality of parts of the integrated surgical system are provided. The method comprises determining the position of at least a first marker being wirelessly tracked by a wireless magnetic tracking system, registering the position of the body part of the subject with an image of the body part of the subject, displaying a registered image of the body part of the subject and at least an image representative of an implant, and receiving a command from a surgeon interface operable by the surgeon.

The present invention relates generally to systems and methods for use in carrying out surgical procedures, and in particular to an integrated orthopaedic surgery system and methods of use thereof, and implants, instruments, computer program code and computer programs for use therein.

Computer aided surgery typically provides for the display of images of body parts and the positions of navigated tools so that the surgeon can use the images to guide them while carrying out the surgical procedure. However, it is typically required to register the image of the patients body part with the actual position of the body part.

Markers detectable by a tracking system can be attached to a body part so that the position of the body part can be tracked, e.g. during a surgical procedure. Such markers are sometime referred to as fiducial markers. A variety of marker types can be used depending on the nature of the tracking system and how signals are generated by the marker and communicated to the tracking system. However, markers are typically provided on some kind of support structure by which the marker is mounted on the body part, such as on the skin, or anchored to bone or another subcutaneous body part or anatomical structure.

For example a surgical sensor is described in U.S. Pat. No. 6,499,488 (Hunter et al.) in which a sensor, which sends signals to a surgical guidance system, is provided in a housing mounted on a surgical screw, or in a hollow part of the screw in lieu of the housing. The surgical screw can be screwed into a bony anatomical structure. Hence, the sensor is attached to a bony anatomical structure by the screw. However, the sensor is still supported by the screw and the sensor is not itself located in the bony structure. Further, an incision is still required in order to attach the sensor to the body part

As indicated above, various methods and systems can be used to track the position of a medical probe or implant inside the body of a subject.

For example, U.S. Pat. Nos. 5,391,199 and 5,443,489 to Ben-Haim, whose disclosures are incorporated herein by reference, describe systems wherein the coordinates of an intrabody probe are determined using one or more field sensors, such as a Hall effect device, coils, or other antennae carried on the probe. Such systems are used for generating three-dimensional location information regarding a medical probe or catheter.

PCT Patent Publication WO 96/05768, and the corresponding U.S. patent application Ser. No. 09/414,875, to Ben-Haim et al. (also published as U.S. Patent Application Publication US 2002/0065455 A1, whose disclosures are incorporated herein by reference, describe a system that generates six-dimensional position and orientation information regarding the tip of a catheter. This system uses a plurality of sensor coils adjacent to a locatable site in the catheter, for example near its distal end, and a plurality of radiator coils fixed in an external reference frame. These coils generate signals in response to magnetic fields generated by the radiator coils, which signals allow for the computation of six location and orientation coordinates.

U.S. Pat. No. 6,239,724 to Doron et al., whose disclosure is incorporated herein by reference, describes a telemetry system for providing spatial positioning information from within a patient's body. The system includes an implantable telemetry unit having (a) a first transducer, for converting a power signal received from outside the body into electrical power for powering the telemetry unit; (b) a second transducer, for receiving a positioning field signal that is received from outside the body; and (c) a third transducer, for transmitting a locating signal to a site outside the body, in response to the positioning field signal.

U.S. patent application Ser. No. 10/029,473 to Govari, published as U.S. Patent Application Publication 2003/0120150, describes apparatus for tracking an object. The apparatus includes a plurality of field generators, which generate electromagnetic fields at different, respective frequencies in a vicinity of the object, and a radio frequency (RF) driver, which radiates a RF driving field toward the object. A wireless transponder is fixed to the object. The transponder includes at least one sensor coil, in which a signal current flows responsive to the electromagnetic fields, and a power coil, which receives the RF driving field and conveys electrical energy from the driving field to power the transponder. The power coil also transmits an output signal responsive to the signal current to a signal receiver, which processes the signal to determine coordinates of the object.

Registration procedures typically require images of the patient to have been acquired previously and so multiple medical procedure at multiple sites are required in order to allow the surgical procedure to be carried out.

Also, different practitioners may be involved in capturing the images and/or carrying out the surgical procedure. Therefore, some of the images that the surgeon would want may not actually have been captured and therefore would not be available to the surgeon. Also the images may be capture some time before the surgery and so may not accurately reflect the current status of the patient.

Further, the surgical practitioner may have little or no control over the information that can be used during the surgical procedure and that information although existing may not be instantly available to the surgeon in the form most useful at any time during the surgical procedure.

Therefore, the present invention addresses deficiencies in surgical systems and method for allowing computer aided surgery to be carried out.

According to a first aspect of the invention, there is provided an integrated surgical system. The integrated surgical system can be used in an orthopaedic operating room to enable a surgeon to carry out a computer aided surgical procedure on a subject or patient.

The integrated surgical system can include a subject support and/or a wireless magnetic tracking system and/or a registration system configured to register the position of the body part of the subject with an image of the body part of the subject and/or a display device and/or a control system which integrates the functionalities of parts of the surgical system and/or a surgeon interface operable by the surgeon to control operation of the integrated surgical system.

The tracking system can generating a magnetic field defining a working volume of the tracking system. The subject support can be located at least partially within the working volume. The tracking system can include a tracking control system configured to track the position of a marker detectable by the tracking system within the working volume and generate a signal indicative of the position of the marker within a reference frame of the tracking system.

The display device can be configured to display a registered image of the body part, or bone, of the subject and/or an image representative of a trackable implant during a computer aided surgical procedure.

The system can comprise a further wireless tracking system. The further wireless tracking system can be an infrared wireless tracking system. The further tracking system can be in communication with the control system and can be configured to generate a signal indicative of the position of a tracked element in the reference frame of the further wireless tracking system.

The display device can be a part of a tracking system control system. The display device can be a touch sensitive display. The display device can be a part of the surgeon interface. A plurality of such display devices can be provided. A separate display device can be provided for each tracking system. Preferably a single display device is provided as a part of the control system for a plurality of tracking systems.

The surgeon interface can include an orientation sensitive device operable by a surgeon to enter control commands. The orientation sensitive device can be a wireless device. The device can be a gyromouse.

The surgeon interface can include a heads up display. The heads up display can be wearable by the surgeon. The heads up display can be configured to display at least a one of the images selected from the group comprising: a captured image of the body part; an image of a model of the body part; a registered image of the body part; a video image of the body part; a representation of an implant; a representation of an instrument; an indication of the planned position of an implant, instrument or incision; and any combination or overlay of the preceding.

The system can further comprise a wall display unit. The wall display unit can be configured to provide a plurality of image regions and/or a single image region. The or each image region can be capable of displaying a different image and/or the image can be a combination of images.

The different images can be selected from the group comprising: a captured image of the body part; an image of a model of the body part; a registered image of the body part; a video image of the body part; a representation of an implant; a representation of an instrument; an indication of the planned position of an implant, instrument or incision; and any combination or overlay of the preceding.

The system can further comprises a surgical site display device. The surgical site display device can be movable. The surgical site display device can include an image display portion and a support. The image display portion can be positionable over the surgical site of the patient in use. The surgical site display device can include an image capturing device having a field of view including the surgical site. The device can generating a surgical site image and the surgical site image can be displayed in the image display portion in registration with the surgical site. The image capturing device can be a video camera. The surgical site image can be, or include, a real time video, or still, image of the surgical site. A further image can be overlayed on the surgical site image. The further image and the surgical site image can be displayed in the image display portion at the same time. The further image can be in registration with the surgical site. The further image can be selected from the group comprising: a captured image of the body part; an image of a model of the body part; a registered image of the body part; a video image of the body part; a representation of an implant; a representation of an instrument; an indication of the planned position of an implant, instrument or incision; and any combination of the preceding.

The system can further comprise an image capturing device which captures real time video, or still, images. The real time video, or still, images can be displayed in real time on at least one display device of the system. Preferably the images are displayed in real time in a one of the image regions of an image wall.

The system can further comprise a surgical light. The surgical light can be suspended and be movable to different positions and orientations with respect to the operating table. An image capturing device can be provided as a part of the surgical light.

A one or a plurality of the parts of the system can be suspended. This reduces the amount of floor space taken up by parts of the system, thereby providing easier and freer access to the patient by the surgeon and other surgical staff.

The system can further comprising an image storage device storing a plurality of captured images of the body part of the subject. The images of the body part can be selected from the group comprising: X-ray images; CT scan images; and X-ray fluoro images. The storage device can be remote or local. A remote storage device can be in communication with the system over a network.

The system can include a model body part storage device. A plurality of generic 3-d models of different body parts, virtual body parts or representations of body parts can be stored. The body parts can be bones. The bones can be selected from the group comprising: a femur; a part of a femur; a femoral head; a pelvis; a part of a pelvis; an acetabulum of a pelvis; a tibia; a part of a tibia; a knee joint; a hip joint; a vertebra; an ankle; fibula; a part of a fibula; a shoulder; a wrist; and an elbow. The storage device can be local or remote. The storage device can be in communication with the system over a network.

An implant image storage device can be provided. The storage device can store 3d images, virtual implants or representations of a plurality of implants useable in the computer aided surgical procedure. The implants can be selected from the group comprising; femoral implants; tibial implants; pelvic implants; spinal implants; prosthetic ankles; prosthetic knees; prosthetic hips; prosthetic shoulders; prosthetic elbows; prosthetic wrists.

An instrument image storage device can be provided. The instrument storage device can store 3d images, virtual instruments or representations of a plurality of instruments useable in the computer aided surgical procedure.

The registration system can include an X-ray or X-ray fluoroscopy registration system. A first and/or second x-ray source can be provided and respective first and/or second detectors associated with the sources can be provided. A source or sources and/or a detector or detectors can be moveable. The source(s) and/or detector(s) can be movable so as to capture images from at least two different directions.

The registration system can be configured to capture at least a first image and a second image of the body part from different directions with the patient on the operating table.

The registration system can includes a first x-ray source and a second x-ray source, a first detector positioned to capture the first image of the body part resulting from the first x-ray source and a second detector positioned to capture the second image of the body part resulting from the second x-ray source. The detectors can be x-ray detectors which generate a digital image or x-ray fluoroscopy detectors.

The first detector and the second detector can be positioned above the subject support the first and second detectors can be suspended. The first x-ray source and the second x-ray source can be positioned below the subject support. The x-ray sources can be located within a floor.

The control system can include a registration control part. The control system can include computer program instructions executable to generate a 3d image of the body part from the first image and second image, to determine the position of the body part in the reference frame of the tracking system and to register the 3d image of the body part with the position of the body part in the reference frame of the tracking system.

The tracking system can include a magnetic field generating subsystem. The position of the magnetic field generating subsystem and/or subject support can be movable so as to change the position and/or orientation of the working volume relative to the subject support. Hence the surgical site can more easily be located within the working volume. A part of the subject support can be movable and/or a part of the magnetic field generating subsystem can be movable. A reference frame on which magnetic field generating coils are mounted can be moved relative to the patient support. The patient support can be moved relative to a reference frame on which magnetic field generating coils are mounted.

The first x-ray source and the second x-ray source can be provided on, in, within or under a floor on which the subject support is located.

The system can include an image handling sub-system. The system can include a video mixing and control subsystem which controls the format, type and display of images on a plurality of different image display parts of the system and/or which receives images from a plurality of different image sources. The image sources can include an endoscope, a video camera, a still camera, a digital camera, an image store, a surgical planning application, a surgical workflow application, an IGS application, and a tracking system or systems. The display devices can include a tracking control system display or displays, an image wall, a heads up display, a surgical site display.

The control system can include computer program instructions providing an orthopaedic surgery workflow program and/or an orthopaedic planning program and/or an image guided surgery program. The image guided surgery program can be configured to implement an orthopaedic procedure at least partially planned by the orthopaedic planning program.

The tracking system can pass or provide data indicating the identity of a marker, or of each of a plurality of markers, being tracked by the tracking system to the control system.

The control system can determine the nature of the element with which the marker is associated. The or each marker can be associated with a bone, an implant, an instrument, or a part of the surgical system, e.g. a part of the registration system or the surgical site display.

The system can further comprise a marker, or a plurality of markers, wirelessly trackable by the tracking system. The marker or markers can be attached to an implant or implants. The marker or markers can be attached to an instrument or instruments. The marker or markers can be attached to a bone or bones. The marker or markers can be attached to a part of the surgical system.

The or each marker can have a housing including a bone anchor for retaining the marker within the bone of the subject. The marker can be hermetically sealed in the housing. The housing can be configured to be percutaneously implantable within the bone of a subject. The or each marker can have a housing and the marker can be hermetically sealed in the housing. The housing can be configured to be secured within or to an implant or part of an implant.

The system can further include a prosthetic joint, or part of a prosthetic joint. The prosthetic joint can comprise a first orthopaedic implant bearing a first marker wirelessly trackable by the tracking system and/or a second orthopaedic implant bearing a second marker wirelessly trackable by the tracking system. A marker can be provided in a wall, stem, pin, peg or bone anchoring part of the orthopaedic implant.

The prosthetic joint can be a knee joint, an ankle joint, a hip joint, an elbow joint, a wrist joint, a hip joint, a shoulder joint, or a spinal joint.

The prosthetic joint can be a prosthetic knee joint, and the first orthopaedic implant can be a femoral component and the second orthopaedic component can be a tibial component. The femoral component can includes a locating pin and the first marker can be located at least partially within the locating pin. The tibial component can includes a keel or anchor and the second marker can be located at least partially within the keel or anchor.

The prosthetic joint can be a prosthetic hip joint, the first orthopaedic implant can be an acetabular component and the second orthopaedic component can be a femoral component. The acetabular component can be a cup and the first marker can be located within a wall of the cup. The marker can be at an apex of the cup. The femoral component can have a body and the second marker can be located at least partially within the body. The second marker can be located at a shoulder of the body or at the tail or stem of the body.

The system can include a plurality of markers wirelessly trackable by the wireless magnetic tracking system. A first of the markers can be configured to be powered by RF induction. The first marker can be implantable in the bone of the subject. A second marker can be configured to be powered by RF induction. The second marker can be attachable to an orthopaedic implant. A third marker can be battery powered. The third is marker can be attachable to an instrument. The instrument can be configured for use in the surgical procedure to prepare for implanting the orthopaedic implant, or for implanting the orthopaedic implant in the body of the subject.

According to a second aspect of the invention, there is provided a dummy or virtual body part for use in training a surgeon to carry out an orthopaedic surgical procedure on a surgical site. The dummy body can comprising an outer layer, an inner volume and a three dimensional formation surrounded by the inner volume. The an outer layer can be of a first material which mimics skin. The inner volume can be of a second material within the outer layer. The second material can mimics interior body tissues, and in particular tissues or structures associated with a joint. The three dimensional formation can be of a third material which mimics bone. The outer layer, inner volume and formation are can be arranged to correspond to a joint of a human body.

The dummy body part can have a first three dimensional formation corresponding to a knee joint and a second three dimensional formation corresponding to a hip joint.

The first material can be a polyurethane elastomer and/or the second material can be a polyurethane elastomer and/or the third material can be a solid foam.

According to a third aspect of the invention, there is provided a method for operating an integrated surgical system to enable a surgeon to carry out a computer aided surgical procedure. The method can include determining the position of at least a first marker being wirelessly tracked by a wireless magnetic tracking system. The position of the body part of the subject can be registered with an image of the body part of the subject. A registered image of the body part of the subject can be displayed on a display device. An image representative of an implant at a current position of the implant relative to the body part can also be displayed on the display device. The images can be displayed during the computer aided surgical procedure. A command can be received from a surgeon interface. Operation of a part of the integrated surgical system can be controlled responsive to the command.

The wireless magnetic tracking system can generates a magnetic field defining a working volume of the tracking system within which the subject support is at least partially located. The position of the marker can be within a reference frame of the tracking system.

The body part and image of the body part can be registered within the reference frame of the tracking system.

The method can further comprise determining the position of a second marker being wirelessly tracked by an infrared wireless tracking system. The position of the second marker can be within a reference frame of the infrared wireless tracking system. The method can further comprising determining the position of the second marker in the reference frame of the wireless magnetic tracking system.

The method can further comprise determining the position of an element to which the marker is attached in the reference frame of the magnetic wireless tracking system. The element can be an instrument, a bone, an implant or a part of the surgical system, such as a part of a registration system or a surgical site display.

The method can further comprise generating an image for display on a heads up display. The image can be supplied to the heads up display. The image can be selected from the group comprising: a captured image of the body part; an image of a model of the body part; a registered image of the body part; a video image of the body part; a representation of an implant; a representation of an instrument; an indication of the planned position of an implant, instrument or incision; and any combination and/or overlay of the preceding.

The method can further comprise generating a plurality of different images for display on a wall display unit. A one of the plurality of images can be supplied for display in an image region of the wall display unit. A different one of the plurality of images can be displayed in each of a plurality of image regions.

The different images can be selected from the group comprising: a captured image of the body part; an image of a model of the body part; a registered image of the body part; a video image of the body part; a representation of an implant; a representation of an instrument; an indication of the planned position of an implant, instrument or incision; and any combination and/or overlay of the preceding.

The method can further comprise capturing a surgical site image of a surgical site. The surgical site image can be supplied to a display device. The display device can be positionable over the surgical site of the patient in use. The surgical site image can be displayed in registration with the surgical site. The surgical site image can be a real time video or still image of the surgical site.

The method can further comprise registering a further image with the position of the surgical site. The further image can be overlayed on the surgical site image. The further image is selected from the group comprising: a captured image of the body part; an image of a model of the body part; a registered image of the body part; a video image of the body part; a representation of an implant; a representation of an instrument; an indication of the planned position of an implant, instrument or incision; and any combination of the preceding.

The method can further comprising capturing real time video images of a surgical site. The real time video images can be supplied for display in real time on at least one display device of the system.

The method can further comprise retrieving and/or receiving an image from an image storage device. The an image can be a one of a plurality of captured images of the body part of the subject. The images of the body part can be selected from the group comprising: X-ray images; CT scan images; ultrasound; and X-ray fluoroscopy images.

The method can further comprise selecting a one of a plurality of generic 3d models of different body parts stored in a storage device. Selecting the 3-d model can be based on a measure of the patient's body part derived from a captured image of the body part. The selected one of the plurality of generic 3d models can be morphed to more closely match the body part of the subject. An image derived from the morphed generic 3d model, or the morphed generic 3d model, can be displayed.

The method can further comprise selecting and/or retrieving a one of a plurality of stored 3d images of a plurality of implants useable in the computer aided surgical procedure. The current orientation and/or position of an implant corresponding to the selected implant can be determined. Selecting the implant image can be based on determining the identity of a marker attached to the implant corresponding to the selected implant image. An image can be generated from the selected 3d image of the implant. The image can correspond to a surgeon's view of the implant for the current orientation of the implant. The image can be displayed at the current position of the implant. The displayed implant image can be registered with a displayed registered image of the body part.

The method can further comprise selecting a one of a plurality of stored 3d images or representations of a plurality of instruments useable in the computer aided surgical procedure. Selecting the instrument image can be based on determining the identity of a marker attached to the instrument corresponding to the selected instrument image. The current orientation and/or position of an instrument corresponding to the selected implant can be determined. An image can be generated from the selected 3d image of the instrument. The image can corresponding to a surgeon's view of the instrument for the current orientation of the instrument. The image can be displayed at the current position of the instrument. The displayed instrument image can be registered with a displayed registered image of the body part.

The method can further comprising capturing a first x-ray or x-ray fluoroscopy image of the body part for a first direction and a second x-ray or x-ray fluoroscopy image of the body part for a second direction, different to the first direction. A 3d image of the body part can be generated from the first image and second image. The position of the body part in the reference frame of the tracking system can be determined. The 3d image of the body part can be registered with the position of the body part in the reference frame of the tracking system.

The position and/or orientation of a captured image of the body part in the reference frame of the tracking system can be used to register the 3d image of the body part and the position of the body part. The position and/or orientation of a captured image can be determined by detecting the position of an image capturing device in the reference frame of the tracking system. The position and/or orientation of a captured image can be determined from a fixed positional and/or orientational relationship of the image capturing device with the reference frame of the tracking system.

The method can further comprise controlling images from different sources and displaying images from different sources on different image display parts of the system.

The method can further comprise displaying a user interface for an orthopaedic surgery workflow program and receiving and processing commands entered via the user interface.

The method can further comprised displaying a user interface for an orthopaedic planning program and receiving and processing orthopaedic planning commands entered via the user interface. At least a part of a surgical plan can be saved. Implant type, implant size and/or implant position selection commands can be received and/or processed.

The method can further comprise displaying a user interface for an orthopaedic image guided surgery program. Commands entered via the user interface can be received and processed to control the image guided surgery procedure.

The method can further comprise generating and displaying images to guide the surgeon to carry out surgical steps. A, some or all of the surgical steps can have been planned by the orthopaedic planning program. The steps can be planned pre-operatively or intra-operatively. Pre-operative planning can be entirely virtual.

The method can further comprising determining the identity of each of a plurality of markers being tracked by the tracking system. The nature of an element with which the marker is associated can be determined for each or all of the plurality of markers.

The nature of the element can be selected from the group comprising: a bone; an implant; an instrument; a tool; and a part of the surgical system.

The method can further comprising determining the current position of a trackable instrument, or all trackable instruments, in the reference frame of the tracking system. Only the current position of an instrument or instruments located within the working volume can be determined.

The method can further comprise determining the current position of a bone, or all bones, in the reference frame of the tracking system. Only the current position of a bone or bones located within the working volume can be determined. The or each bone can have a marker implanted therein.

The method can further comprise determining the position in the reference frame of the tracking system of a first orthopaedic implant bearing a first marker wirelessly trackable by the tracking system. The position in the reference frame of the tracking system of a second orthopaedic implant bearing a second marker wirelessly trackable by the tracking system can be determined. The position in the reference frame of the tracking system of all marked orthopaedic implants can be determined. Only the current position of an implant or implants located within the working volume can be determined

The first orthopaedic implant can be a femoral component of a prosthetic knee joint and/or the second orthopaedic component can be a tibial component of a prosthetic knee joint. The first orthopaedic implant can be an acetabular component of a hip joint and/or the second orthopaedic component can be a femoral component of a hip joint.

According to a fourth aspect of the invention, there is provided computer program code executable by a data processing device to provide the method of the third aspect of the invention. There is also provided a computer readable medium bearing computer program code according to the fourth aspect of the invention.

According to a fifth aspect of the invention, there is provided a wirelessly trackable prosthetic joint. The prosthetic joint can comprise a first component bearing a first wirelessly trackable marker and/or a second component bearing a second wirelessly trackable marker. The first wirelessly trackable marker and/or the second wirelessly trackable marker can each be hermetically sealed.

The first and/or second wirelessly trackable marker can be configured to be powered by RF induction.

The first wirelessly trackable marker and/or the second wirelessly trackable marker can each be hermetically sealed in an encapsulant and/or in a housing. The housing can include at least a ceramic part.

The first and/or second wirelessly trackable marker can be magnetically wirelessly trackable.

The first and/or second wirelessly trackable marker can be located within a wall, stem, locating formation, pin, keel or anchor part of an implant component. The marker can be enclosed within any of the preceding parts of the implant component.

The first and/or second wirelessly trackable marker can be wirelessly trackable with the first component and/or the second component implanted subcutaneously in the body of a subject. That is the makers can be trackable through the patient's skin after the surgical wound has been closed and without the marker being exposed by the skin.

The joint can be a prosthetic knee, a prosthetic hip, a prosthetic ankle, a prosthetic wrist, a prosthetic elbow, a prosthetic shoulder or a prosthetic spinal part or joint.

The joint can be a prosthetic knee. The joint can be a uni-condylar prosthetic knee. The first component can be a femoral component. The femoral component can have a femur engaging surface and a bearing surface corresponding to a single condyle of the femur. The second component can be a tibial component. The tibial component can have a tibia engaging surface and a bearing on an opposed side. The bearing can be configured to engage with a single condyle bearing surface only of the femoral component as the prosthetic knee is articulated.

The femoral component can includes a location pin. The location pin can extend from the femur engaging surface. The location pin can have a cavity therein in which the marker is partially located or wholly located. The marker can be enclosed within the location pin.

The femoral component can be configured with at least a first sensor coil of the marker aligned or parallel with a principal axis of the body part. The principal axis can be the longitudinal axis of the femur.

The tibial component can include a keel or anchor part for engaging in the tibia in use. The marker can be located at least partially in the keel or anchor part.

The tibial component can be configured with at least a first sensor coil of the marker aligned with a principal axis of the body part. The principal axis can be an anterior-posterior axis or direction of the tibia.

The joint can be a hip joint. The first component can be an acetabular component. The second component can be a femoral component. The femoral component can be or include a stem part.

The first marker can comprise a housing defining a cavity and a marker located within the cavity. The cavity can have three parts. A first part can receive a sensor coil. A second part can receive control circuitry. A third part can receive an RF power induction coil.

The acetabular component can have a wall and the acetabular marker can be located within the wall of the acetabular component.

The housing can have a convex outer surface and a concave inner surface. The acetabular component can have a convex outer surface and a concave inner surface. The outer surface of the housing can smoothly continues the outer surface of the acetabular component. The inner surface of the housing can smoothly continue the inner surface of the acetabular component. The inner surfaces of the housing and/or acetabular component can be highly polished to provide an articulate surface.

The femoral component can defines a cavity therein and the second marker can be located partially or wholly in the cavity. The marker can be enclosed in the cavity.

According to a sixth aspect of the invention, there is provided a kit of parts for use in a computer aided orthopaedic surgical procedure. The kit includes a first percutaneously implantable marker for implanting in a first bone associated with a joint to be replaced and a prosthetic joint according to the fifth aspect of the invention. A second percutaneously implantable marker for implanting in a second bone associated with the joint to be replaced can also be provided.

The kit can further comprise an instrument or instrument assembly for injecting the first and/or second markers through the skin of the patient so as to implant the markers in the bone or bones of the patient.

According to a seventh aspect of the invention, there is provided a computer implemented method for carrying out an orthopaedic surgical procedure. The procedure can include implanting a first orthopaedic implant bearing a first marker magnetically wirelessly trackable by a tracking system and/or a second orthopaedic implant bearing a second marker magnetically wirelessly trackable by the tracking system in a body of a subject. The method can include creating a surgical plan defining the intended implantation positions for the first and/or second orthopaedic implants. An image of a part of the body of the subject can be registered with the position of the part of the body of the subject in the reference frame of the tracking system. The surgical plan can be registered with the tracking system. The current positions of the first and/or second orthopaedic implants are determined. A first image representing the part of the body of the patient, a second image representing the current position of the first orthopaedic implant and/or a third image representing the current position of the second orthopaedic implant can be displayed. An indication of the planned positions of the first and/or second orthopaedic implants derived from the surgical plan can also be displayed.

According to a eighth aspect of the invention, there is provided a method for carrying out an orthopaedic computer aided surgery procedure on a body of a subject in an operating room. The method can include planning the intended position of a first orthopaedic implant wirelessly magnetically trackable by a tracking system having a reference frame. A part of the body of the subject in the operating room can be registered. An image guided surgery system can be used to determine an implantation position of the first orthopaedic implant in the part of the body. The orthopaedic implant can be implanted at the implantation position.

The method can further comprise percutaneously implanting at least a first sensor wirelessly magnetically trackable by the tracking system in a bone of the part of the body.

The first sensor can be implanted prior to locating the body in the operating room.

The first sensor can be implanted with the body in the operating room.

The first sensor can be implanted prior to planning the intended position of the first orthopaedic implant.

Registering a part of the body can occur before planning the intended position of the first orthopaedic implant. Registering a part of the body can occur after planning the intended position of the first orthopaedic implant.

Planning the intended position can be carried out virtually.

The method can further comprising taking first and second x-ray, or x-ray fluoroscopic, images of the part in the operating room from different directions. The intended position of the first orthopaedic implant can be planned using a 3d model of the body part derived from the first and second images. Preferably the first and second images are from directions approximately 90E apart.

The first and second x-ray, or x-ray fluoroscopic, images of the part can be taken without moving the patient in the operating room. The method can include moving an x-ray source and/or an x-ray, or x-ray fluoroscopy, detector.

The method can further comprise visually assessing the performance of the implanted first orthopaedic implant in the operating room by viewing a real time representation of the position of the implant or implants and/or the part of the body immediately after implantation and before or after closing the surgical wound.

The method can further comprise percutaneously removing a marker wirelessly magnetically trackable by the tracking system from within a bone of the body part.

Preferred features of a one of the aspects of the invention can also be counterpart preferred features of other aspects of the invention mutatis mutandis.

The present invention will be more fully understood from the following detailed description of the embodiments thereof, taken together with the drawings in which:

FIG. 1 shows a schematic block diagram illustrating an orthopaedic operating room according to the invention;

FIG. 2 shows a perspective view of the orthopaedic operating room illustrated in FIG. 1;

FIG. 3 shows a schematic block diagram of the orthopaedic operating room shown in FIG. 2;

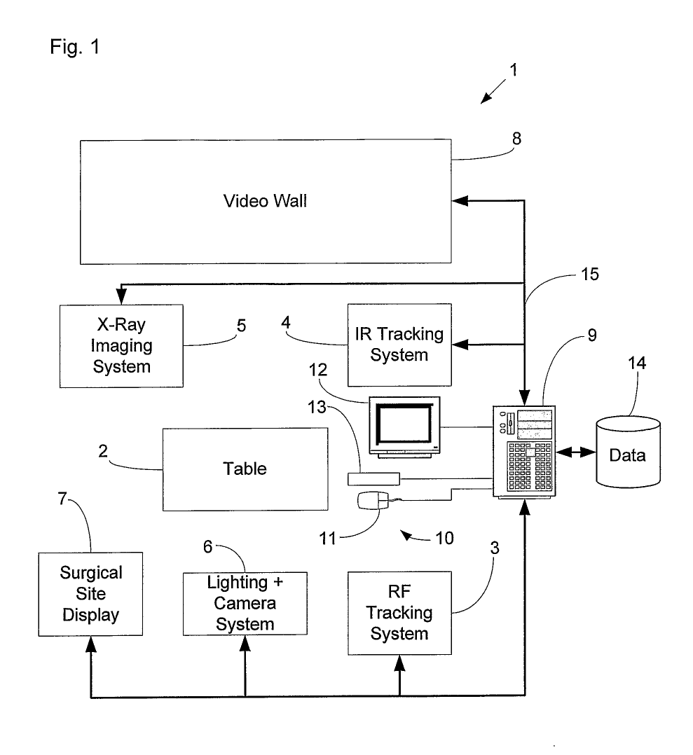

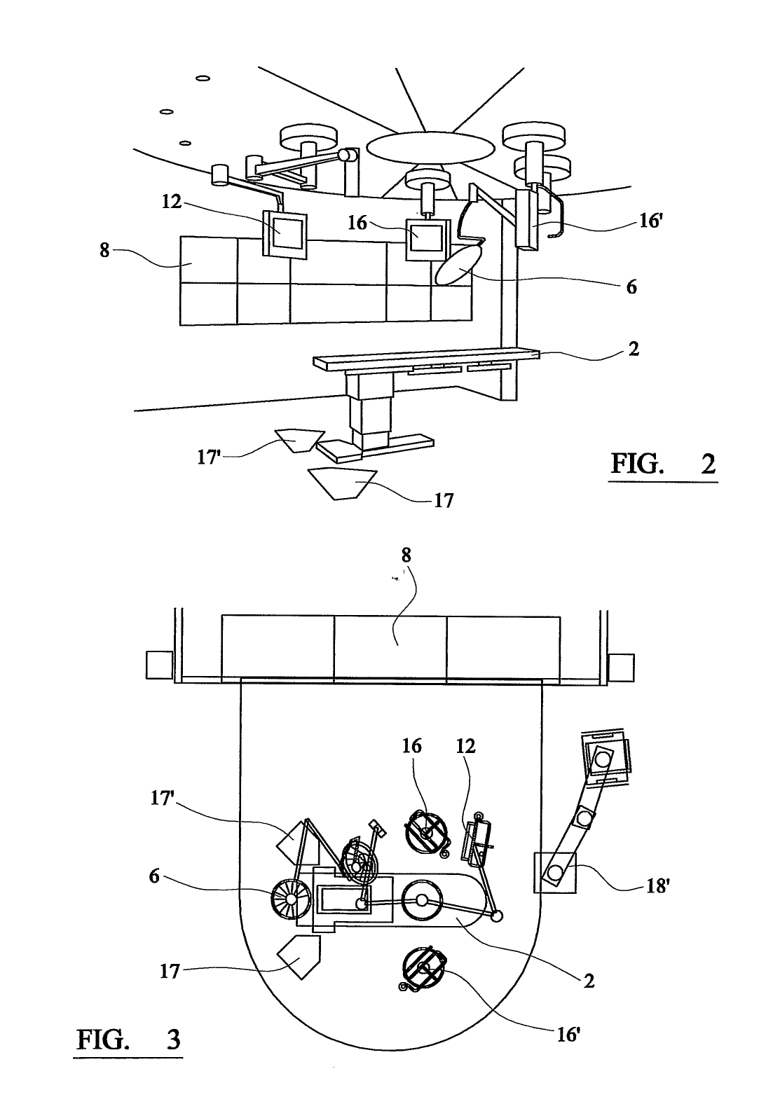

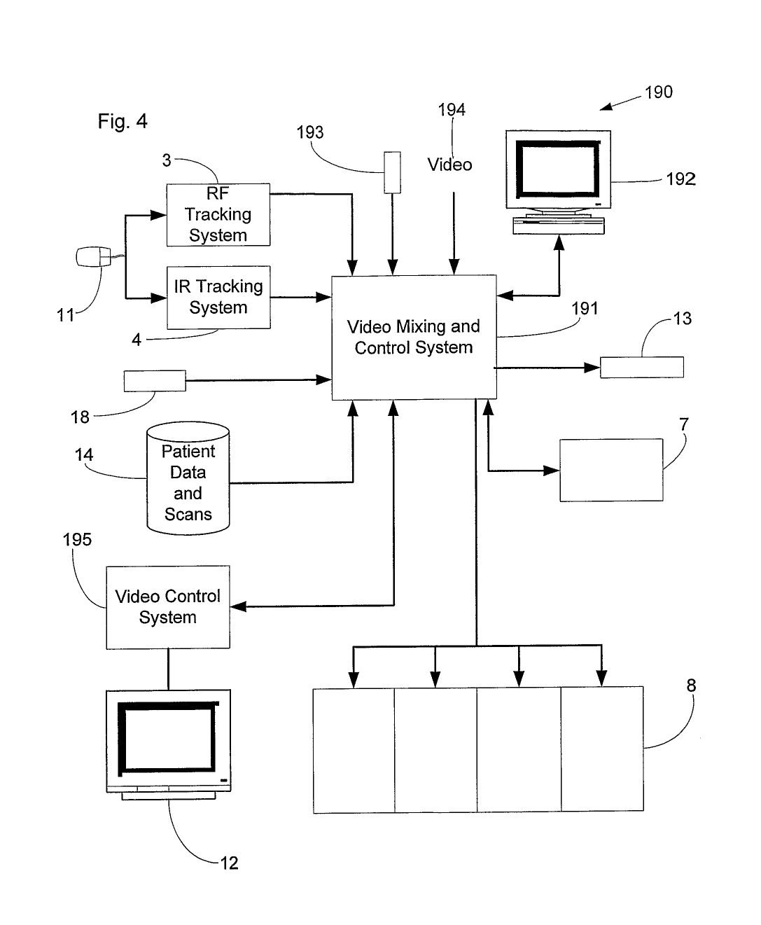

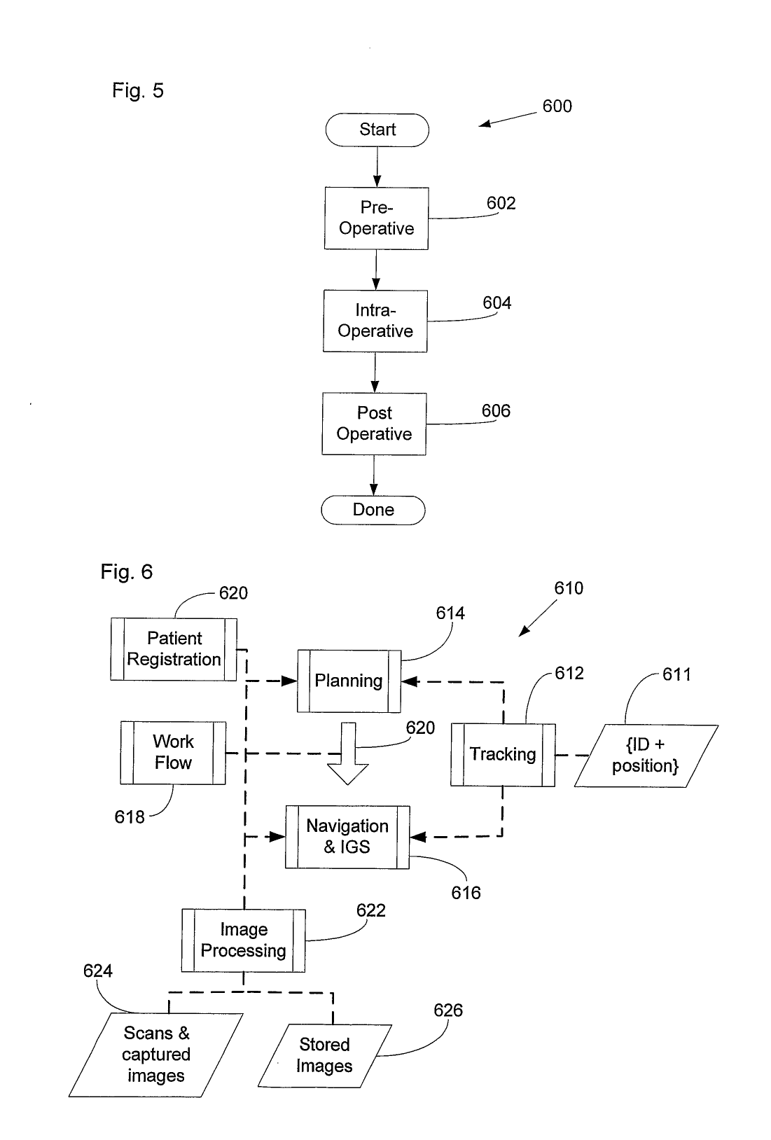

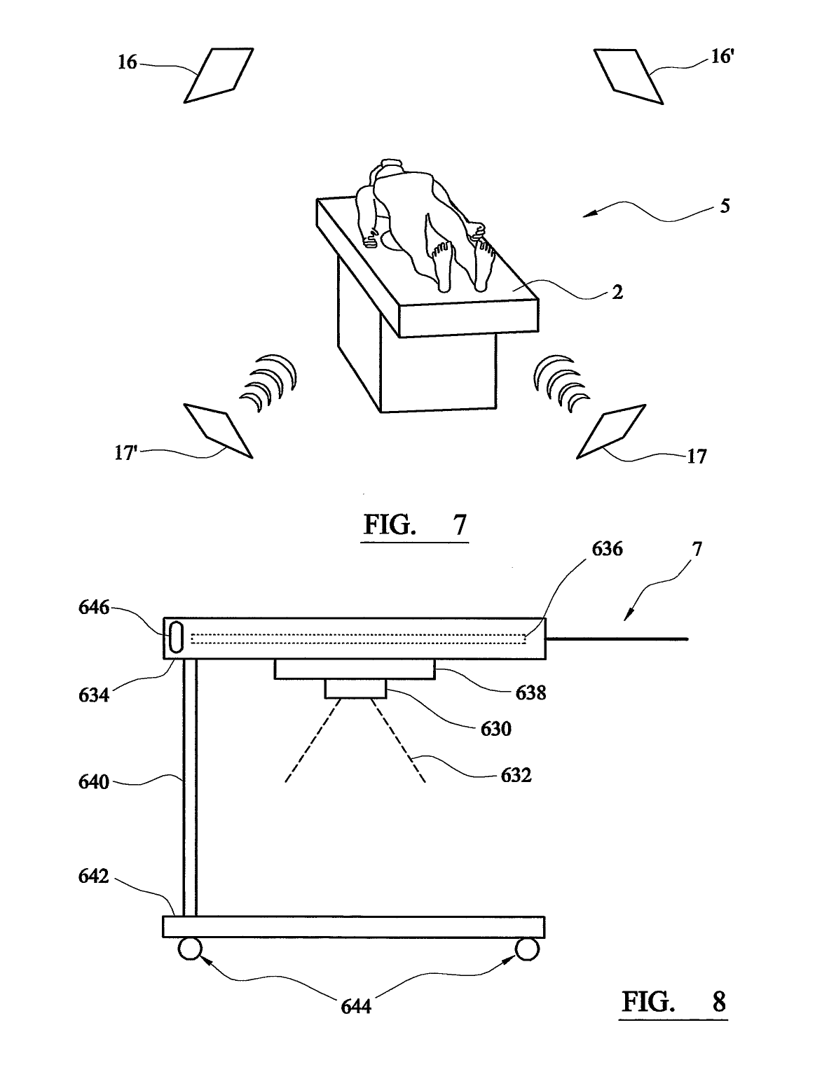

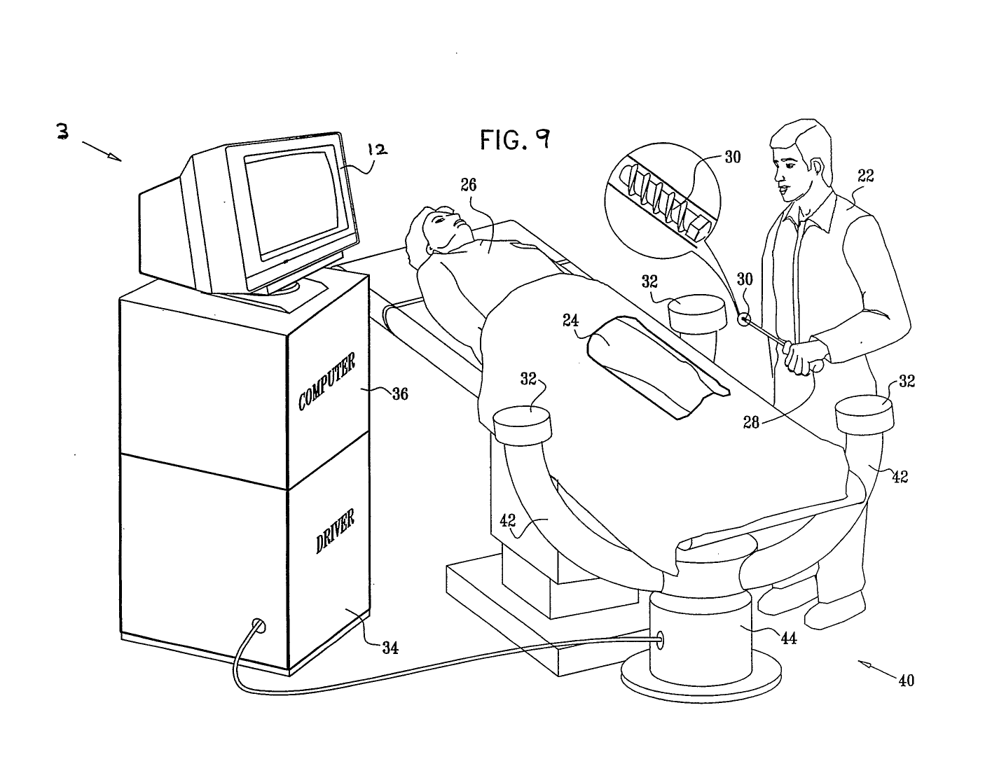

FIG. 4 shows a schematic block diagram of an image control subsystem of the operating room;

FIG. 5 shows a high level flow chart illustrating phases of use of the orthopaedic operating room;

FIG. 6 shows a schematic representation of a software architecture of the orthopaedic operating room;

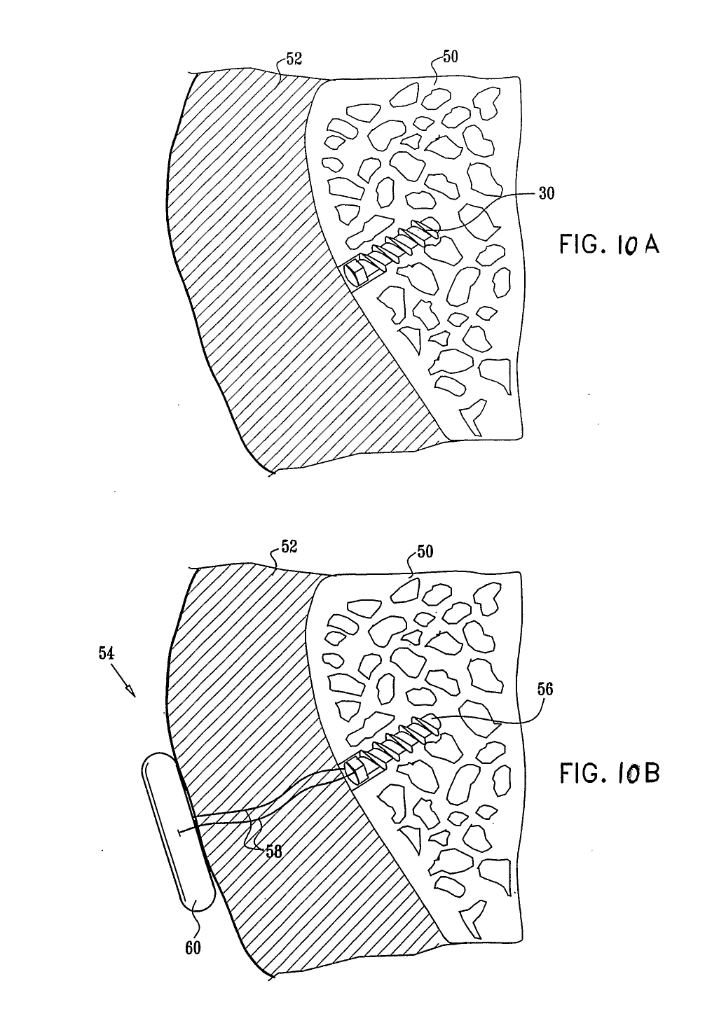

FIG. 7 shows a perspective view of an X-ray imaging part of the orthopaedic operating room;

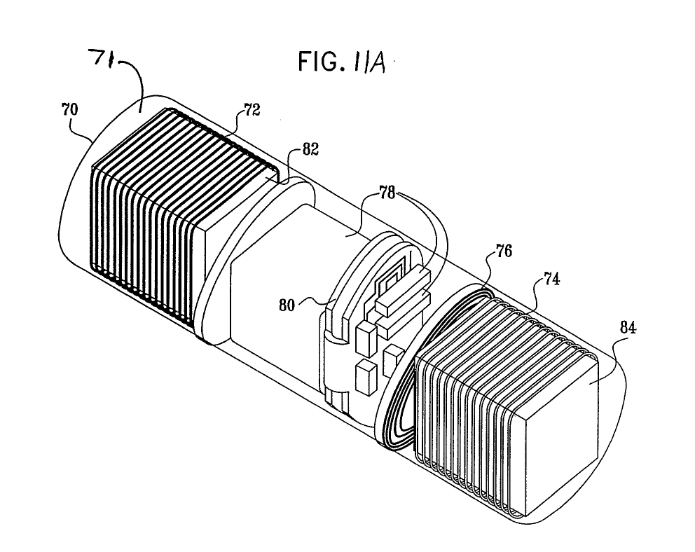

FIG. 8 shows a schematic view of a real time surgical site display part of the orthopaedic operating room;

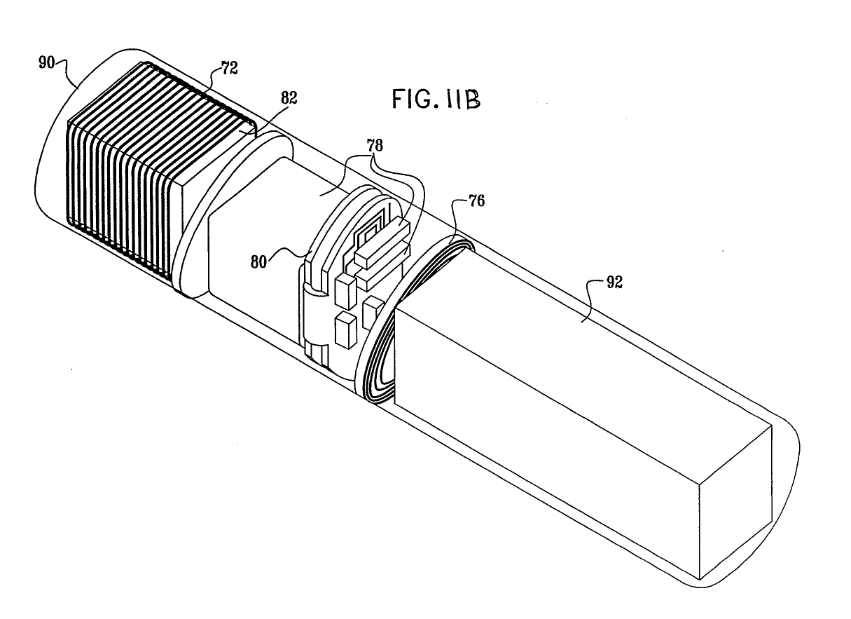

FIG. 9 shows a schematic, pictorial illustration including a magnetic tracking sub system of the orthopaedic operating room;

FIGS. 10A and 10B are schematic, partly sectional illustrations, showing insertion of an embodiment of an implantable marker in the bone of a patient to be treated in the orthopaedic operating room;

FIGS. 11A and 11B are schematic, pictorial illustrations showing details of wireless position sensor or marker parts of an implantable marker, an instrument marker and an implant marker;

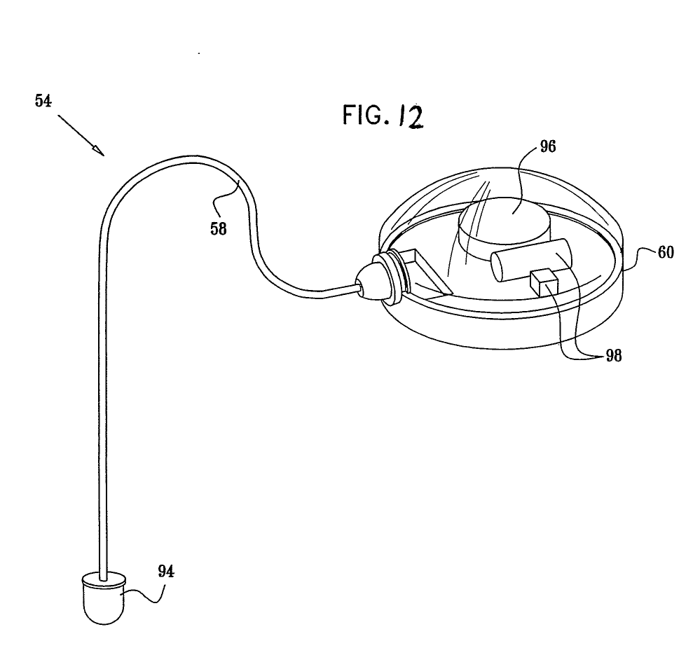

FIG. 12 is a schematic, pictorial illustration showing details of a two-part position sensor or marker;

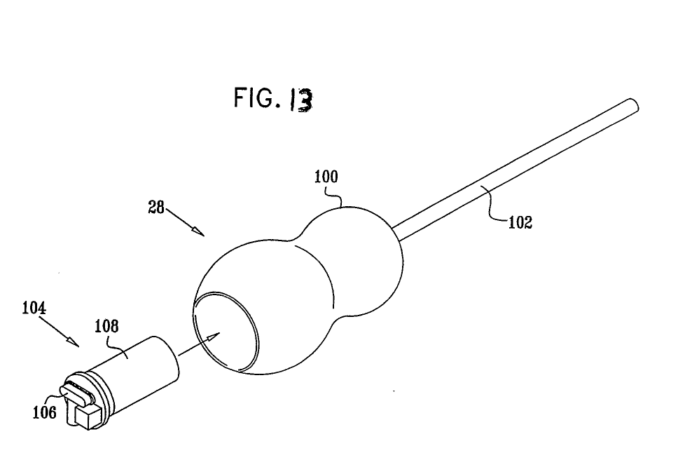

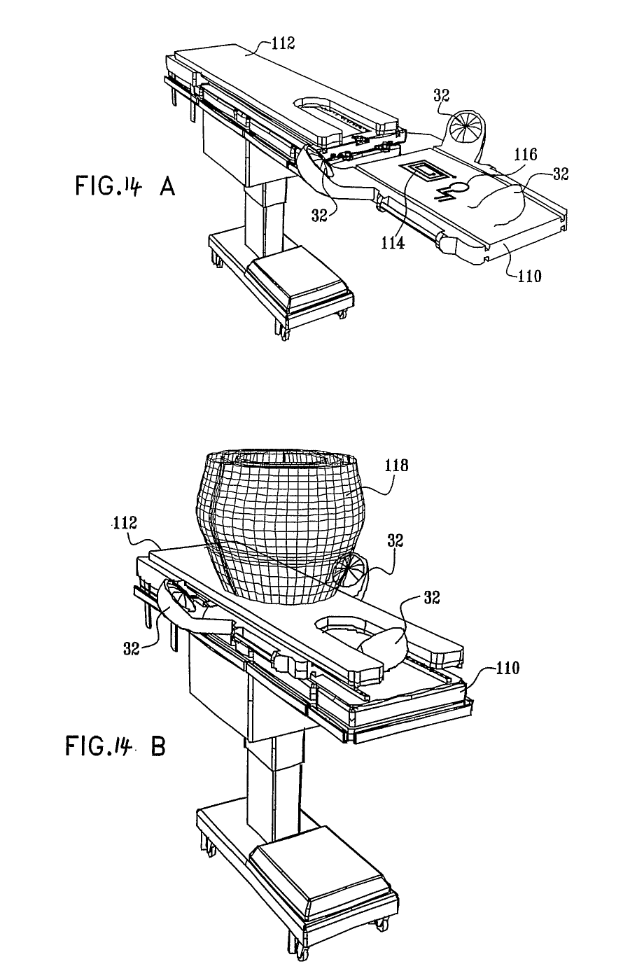

FIG. 13 is a schematic, pictorial illustration showing a surgical tool and a marker used to track coordinates of the tool in the orthopaedic operating room;

FIG. 14A is a schematic, pictorial illustration showing an operating table and a location

FIG. 14B is a schematic, pictorial illustration showing the location pad of FIG. 14A after insertion into the operating table, and showing the working volume of the location pad;

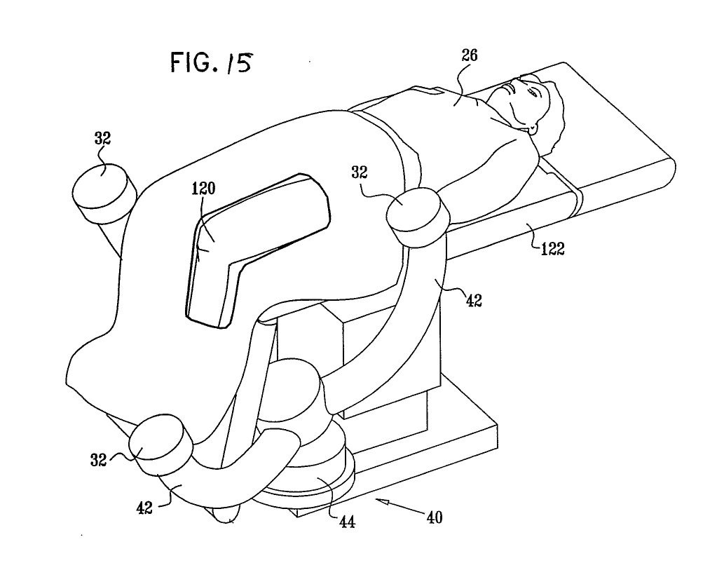

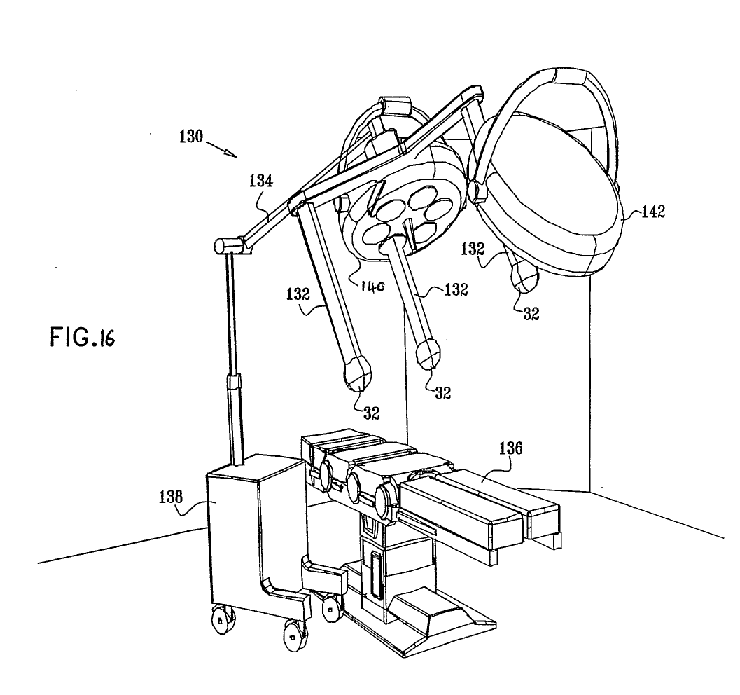

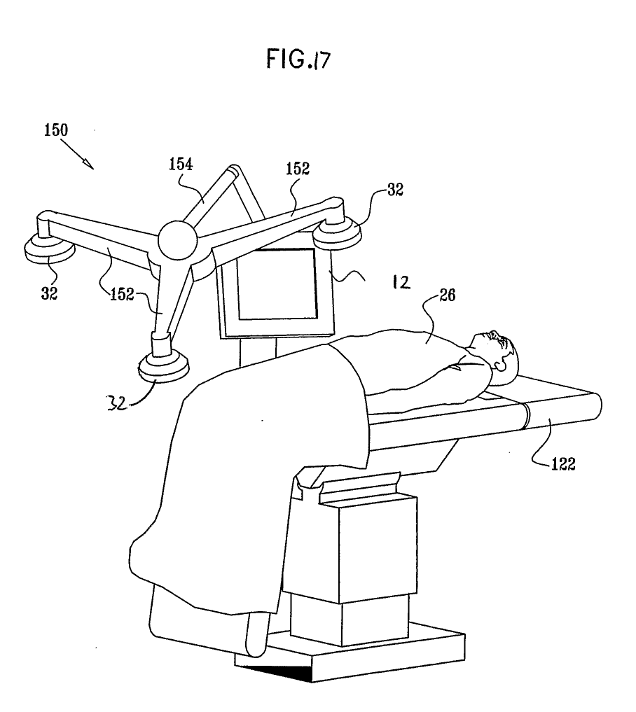

FIG. 15 is a schematic, pictorial illustration showing adjustment of a part of the magnetic tracking system for use in a knee operation;

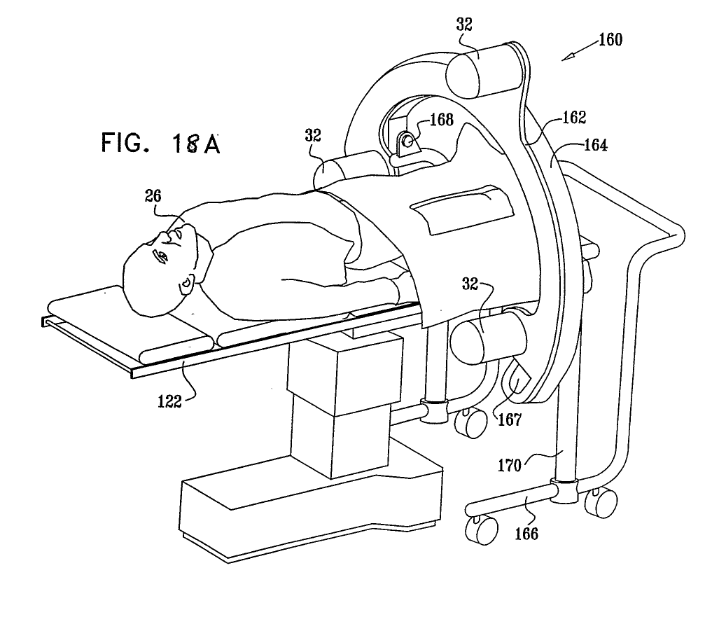

FIG. 16 is a schematic, pictorial illustration of a further magnetic tracking subsystem part and operating table part of the orthopaedic operating room;

FIG. 17 is a schematic, pictorial illustration of a further magnetic tracking subsystem and operating table part of the orthopaedic operating room;

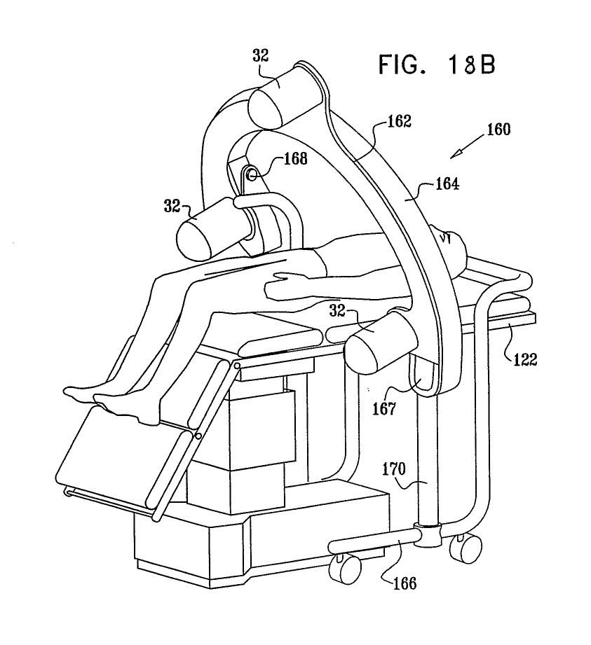

FIGS. 18A and 18B are a schematic, pictorial illustrations of a further magnetic tracking subsystem and operating table part of the orthopaedic operating room;

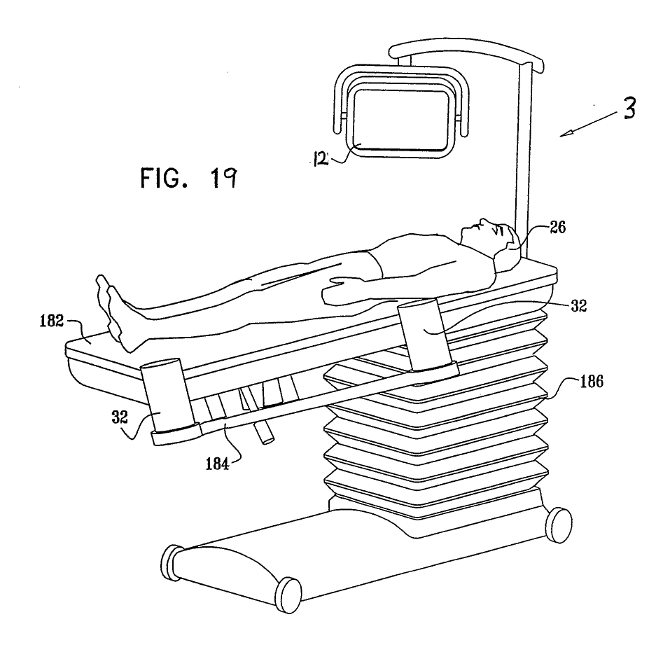

FIG. 19 is a schematic, pictorial illustration of a further magnetic tracking subsystem and operating table part of the orthopaedic operating room;

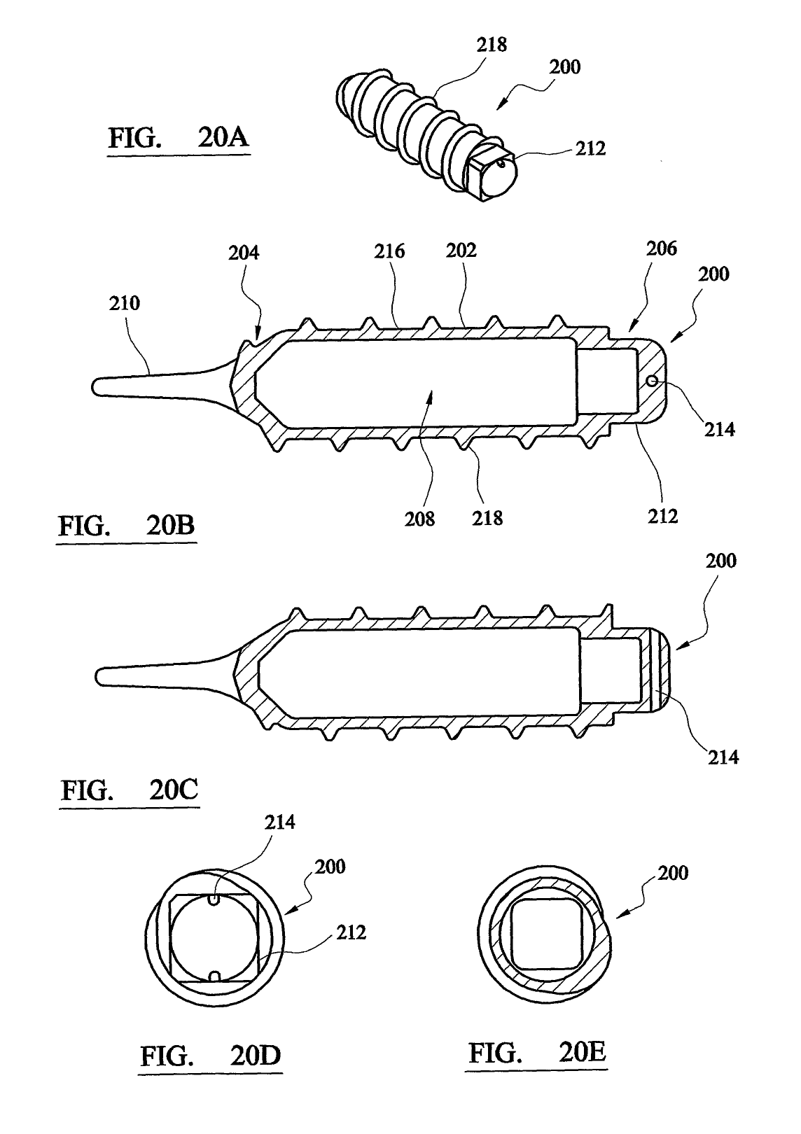

FIGS. 20A, 20B, 20C, 20D and 20E respectively show a perspective view, two longitudinal cross sectional views, a first end view and a transverse cross sectional view of a housing part of an implantable marker for use with the magnetic tracking subsystem of the orthopaedic operating room;

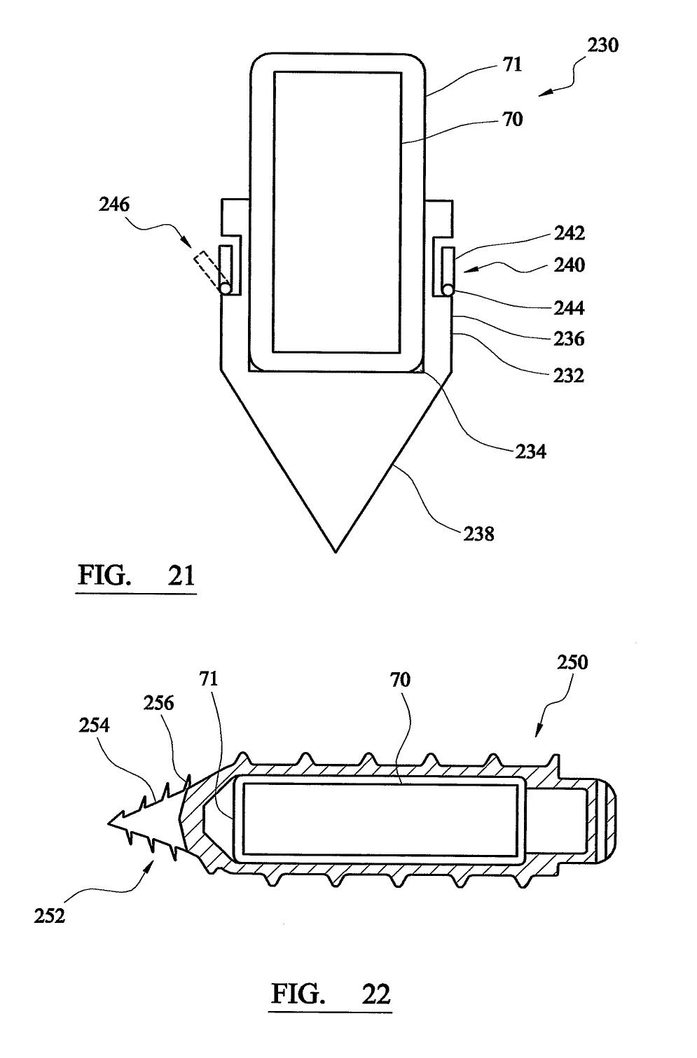

FIG. 21 shows a schematic cross sectional view of a further implantable marker;

FIG. 22 shows a schematic cross sectional view of a further implantable marker;

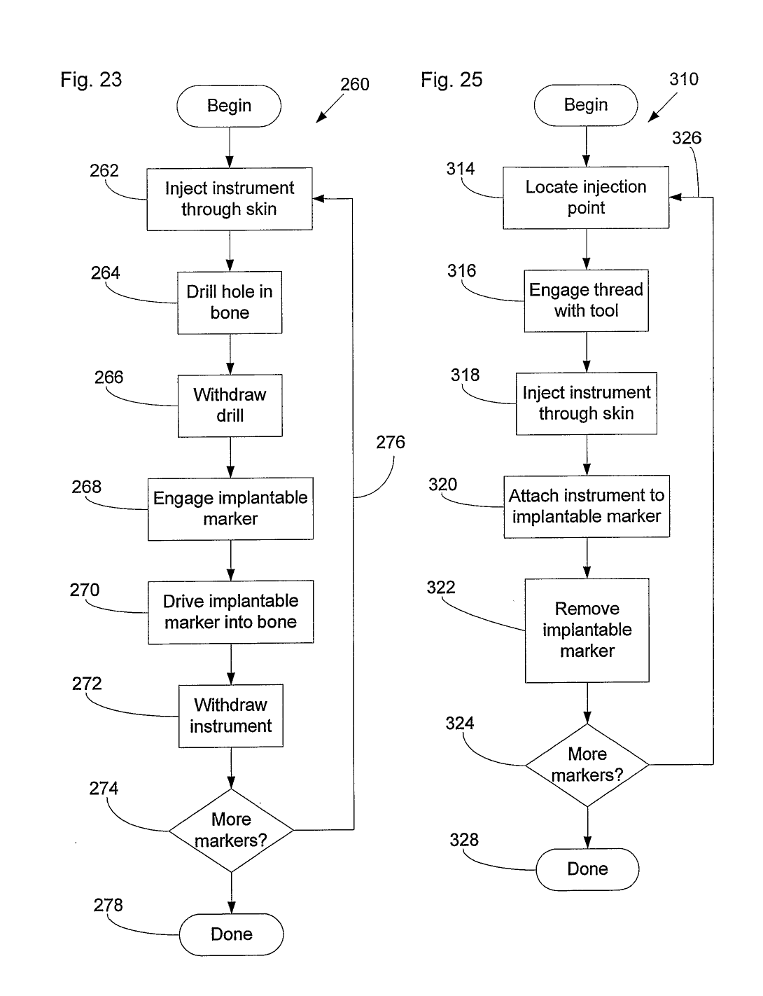

FIG. 23 shows a flow chart illustrating a pre-operative method for implanting the implantable marker through the skin of a patient to be treated in the orthopaedic operating room;

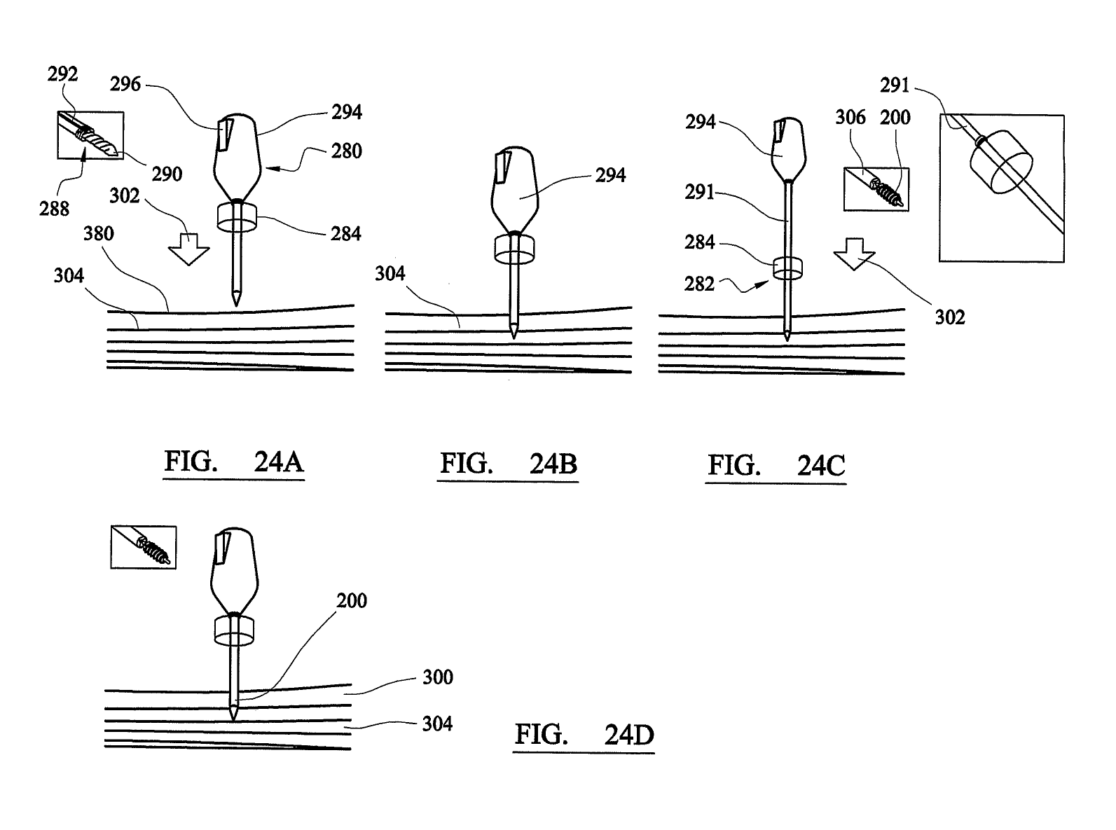

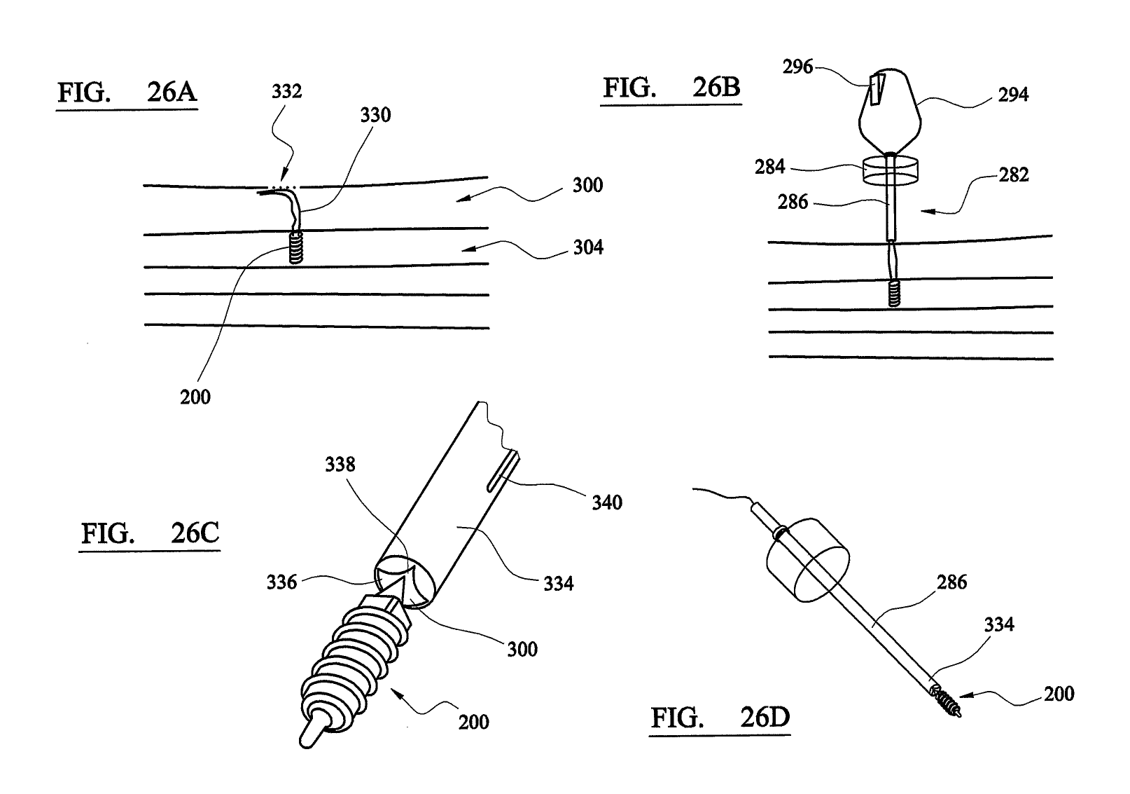

FIGS. 24A-24D show pictorial representations illustrating parts of the method of FIG. 23;

FIG. 25 shows a flow chart illustrating a post-operative method for removing an implantable marker through the skin of the patient;

FIGS. 26A-26D show pictorial representations illustrating parts of the method of FIG. 25;

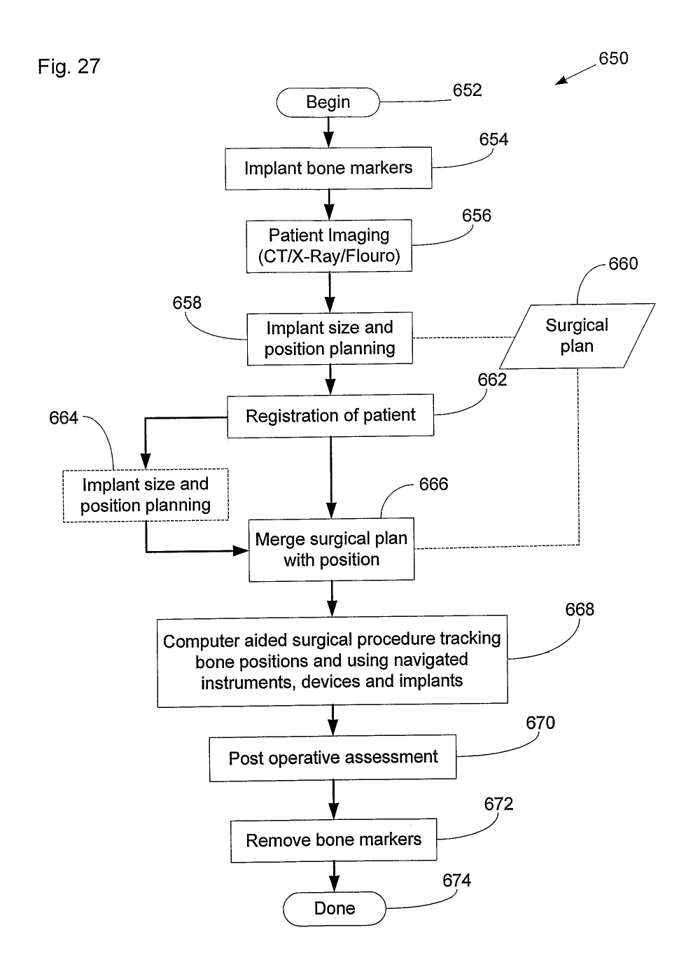

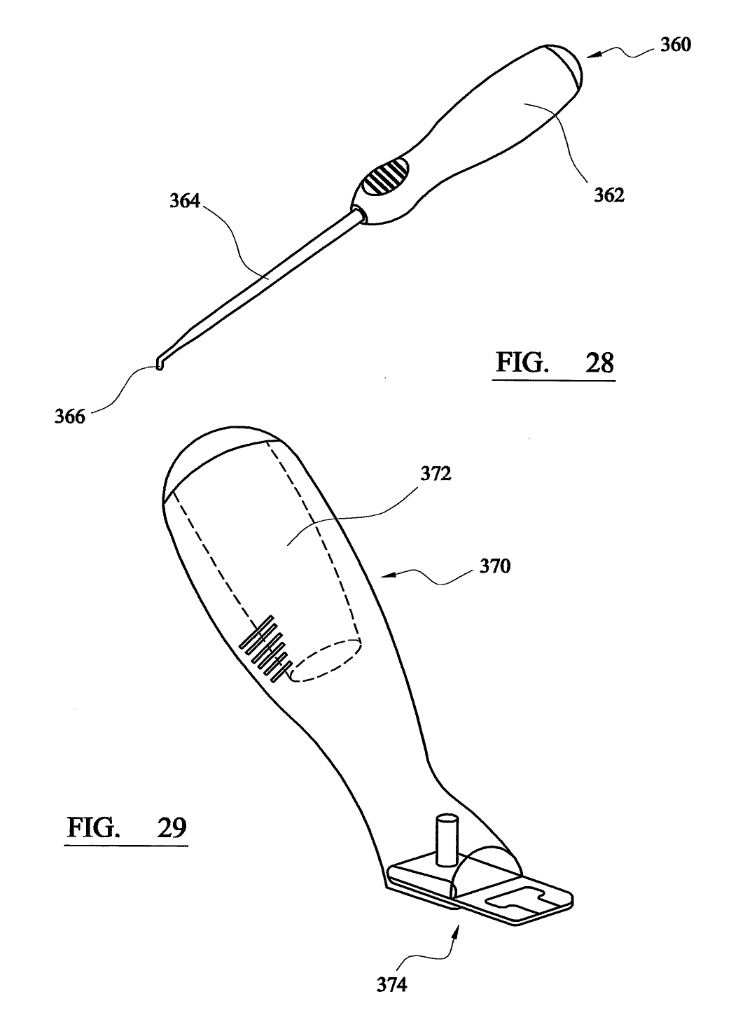

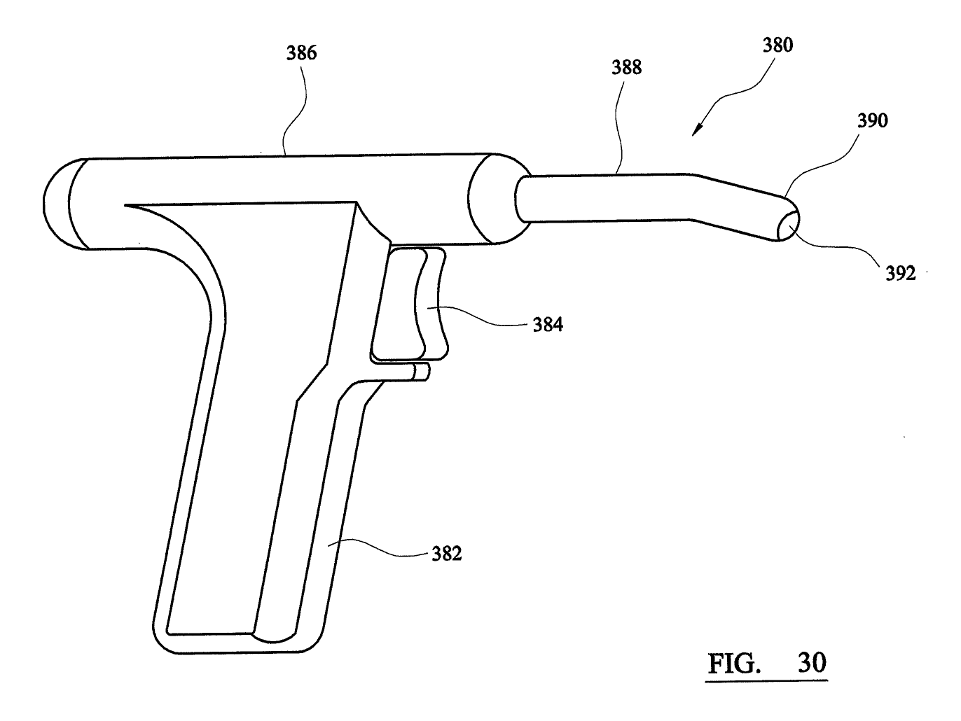

FIG. 27 shows a high level flow chart illustrating a computer aided surgical method according to an embodiment of the invention and a method of using the orthopaedic operating room according to an embodiment of the invention;

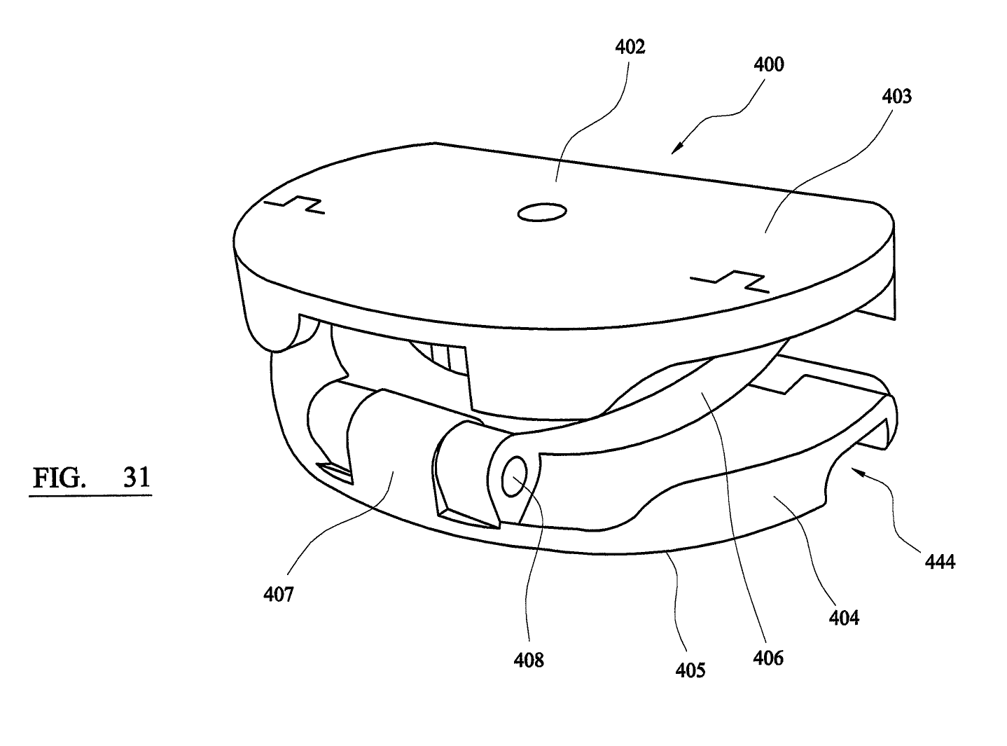

FIG. 28 shows a schematic perspective view of a marked pointer tool for use in the orthopaedic operating room;

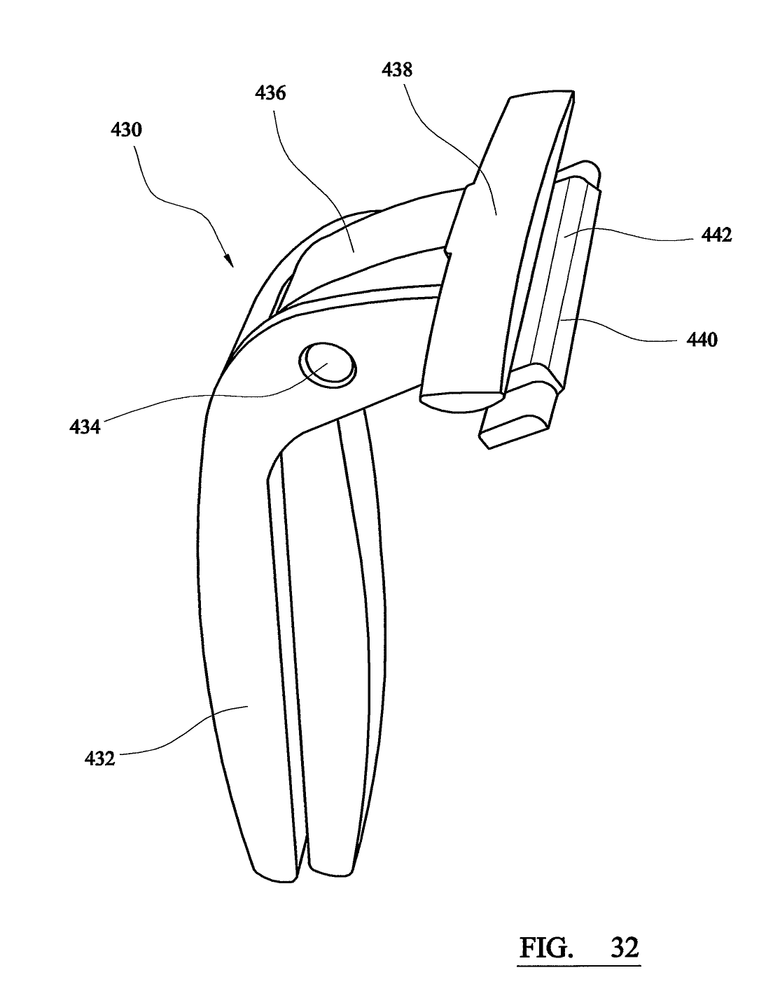

FIG. 29 shows a schematic perspective view of a marked plane tool for use in the orthopaedic operating room

FIG. 30 shows a schematic perspective view of a marked burr tool for use in the orthopaedic operating room;

FIG. 31 shows a schematic perspective view of a tensor device for use in the orthopaedic operating room;

FIG. 32 shows a schematic perspective view of a compression tool for use with the tensor device shown in FIG. 31 in the orthopaedic operating room;

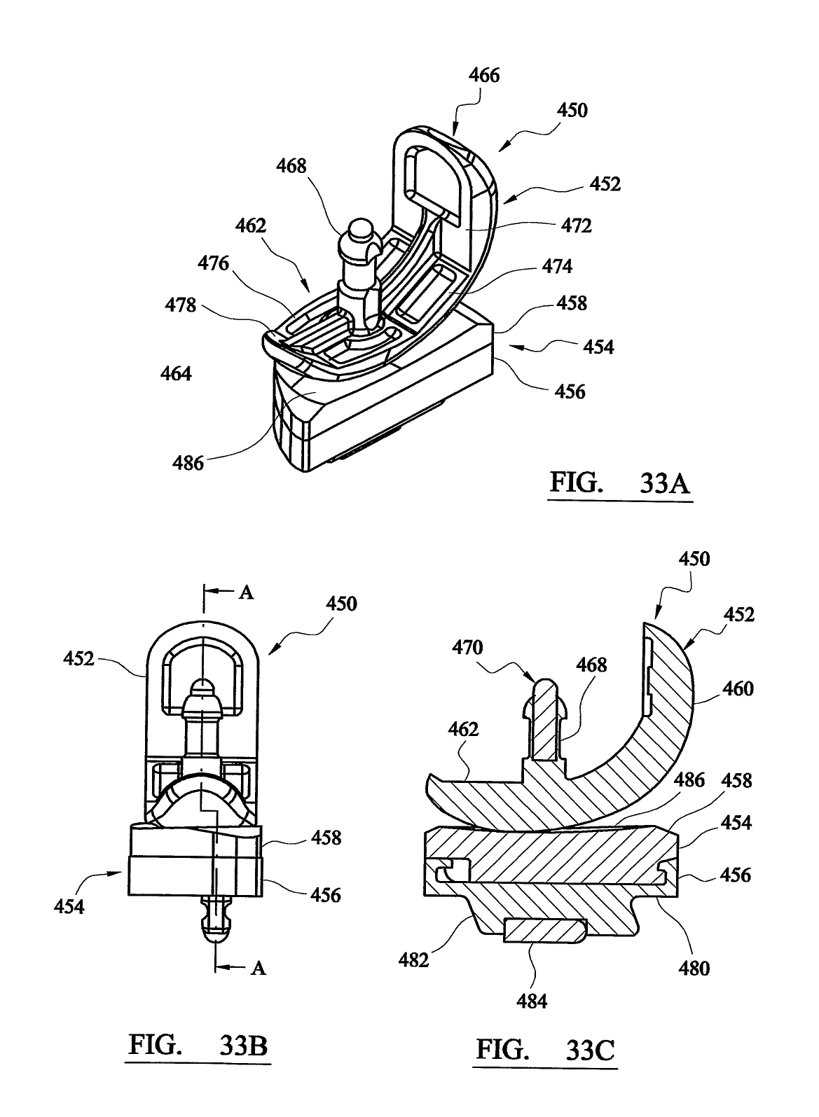

FIGS. 33A, 33B and 33C respectively show a perspective, an end and a cross sectional view along line AA of FIG. 33B of a navigable unicondyle prosthetic knee implant according to an embodiment of the invention;

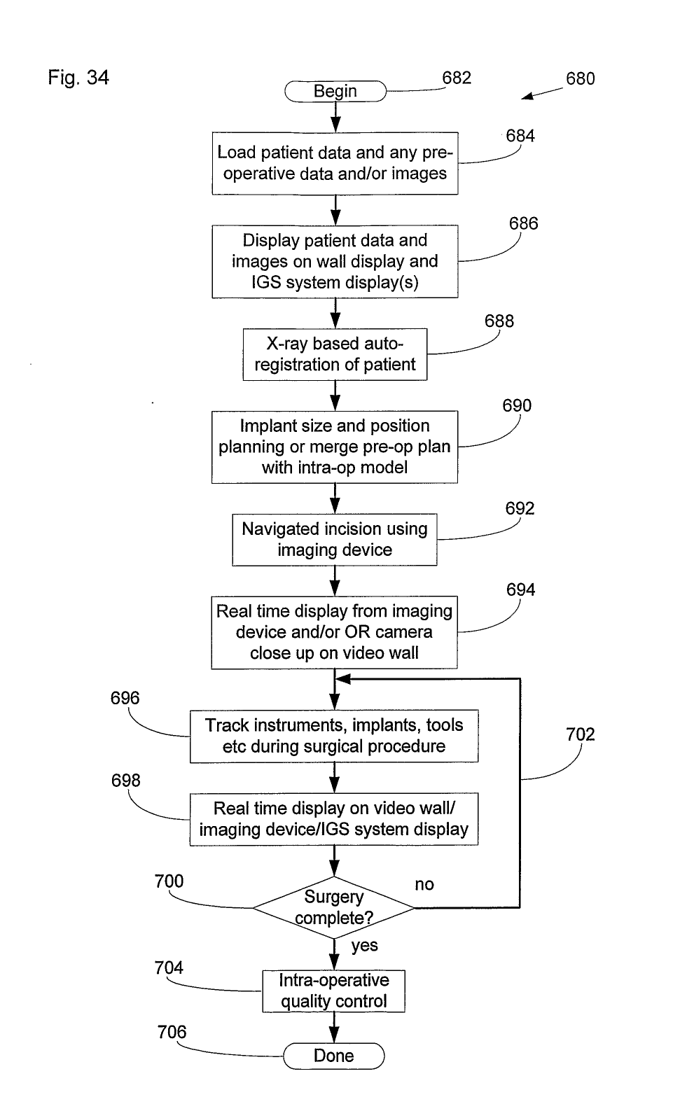

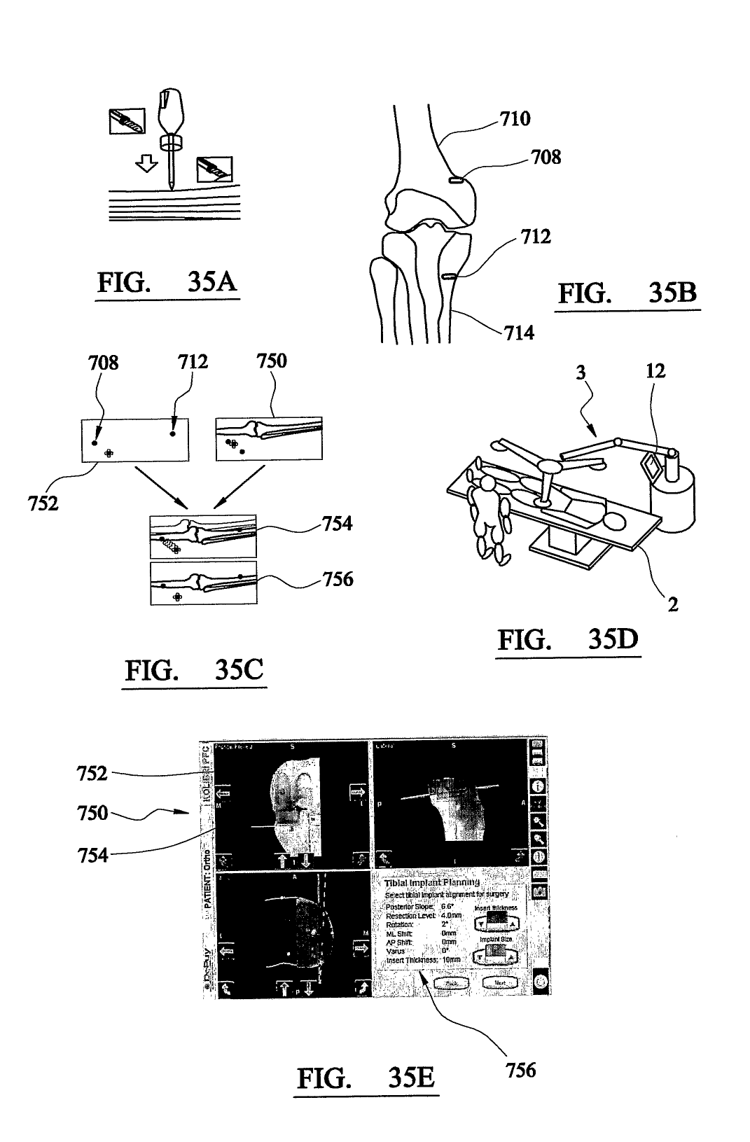

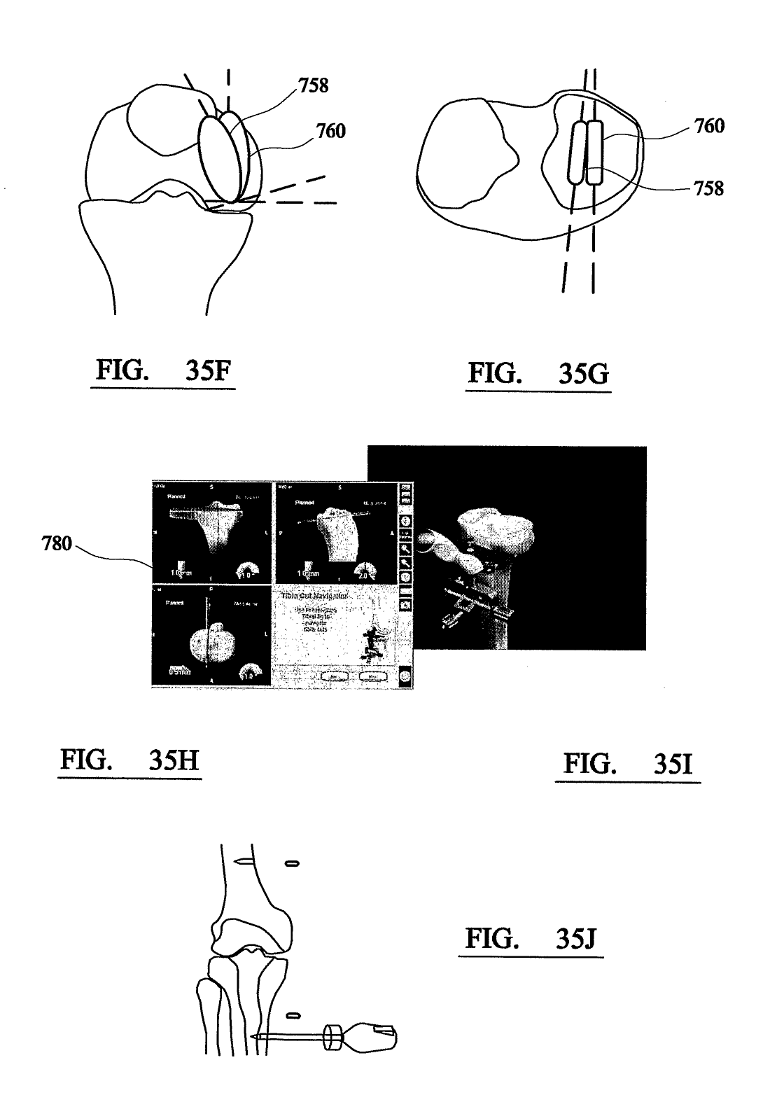

FIG. 34 is a flow chart illustrating a computer aided surgical method for carrying out a knee replacement operation using the orthopaedic operating room according to an embodiment of the invention;

FIGS. 35A-34J are pictorial representations of some of the steps carried out in FIG. 34;

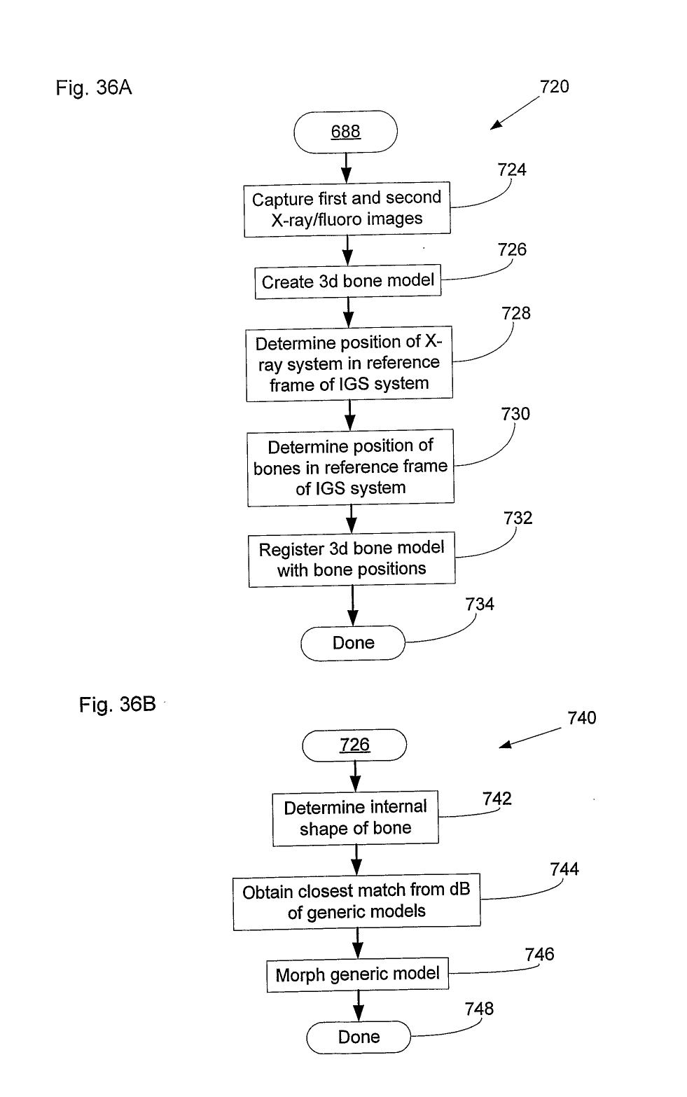

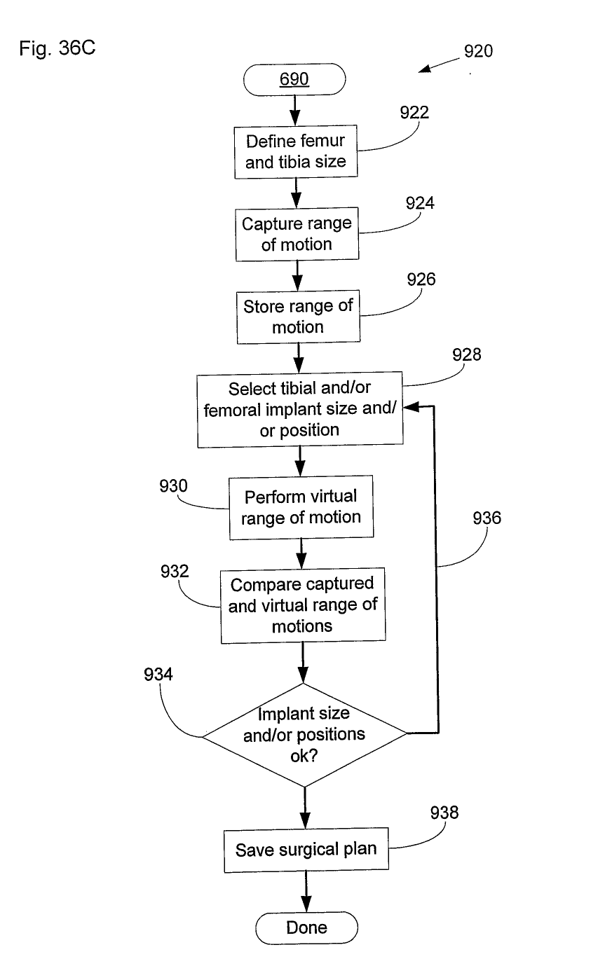

FIG. 36A shows a flow chart illustrating an X-ray based auto-registration method part of the method shown in FIG. 34;

FIG. 36B shows a flow chart illustrating a 3d model creation part of the method illustrated in FIG. 36A;

FIG. 36C shows a flow chart illustrating a computer aided orthopaedic planning method part of the method shown in FIG. 34;

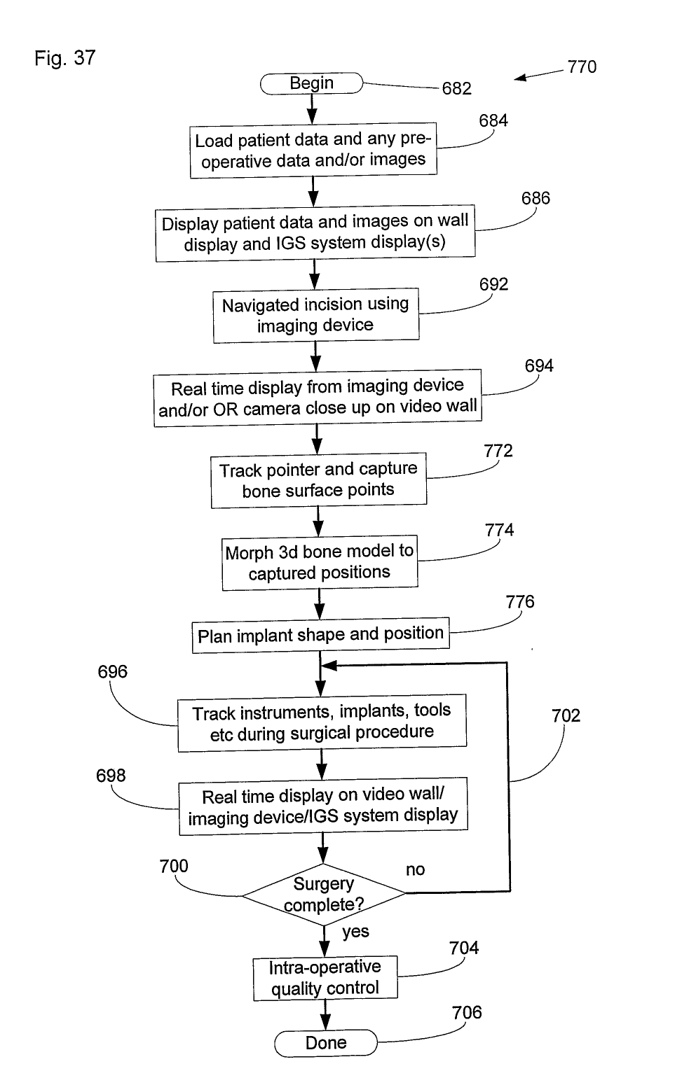

FIG. 37 is a flow chart illustrating a captured body image free version of the computer aided surgical method illustrated in FIG. 34;

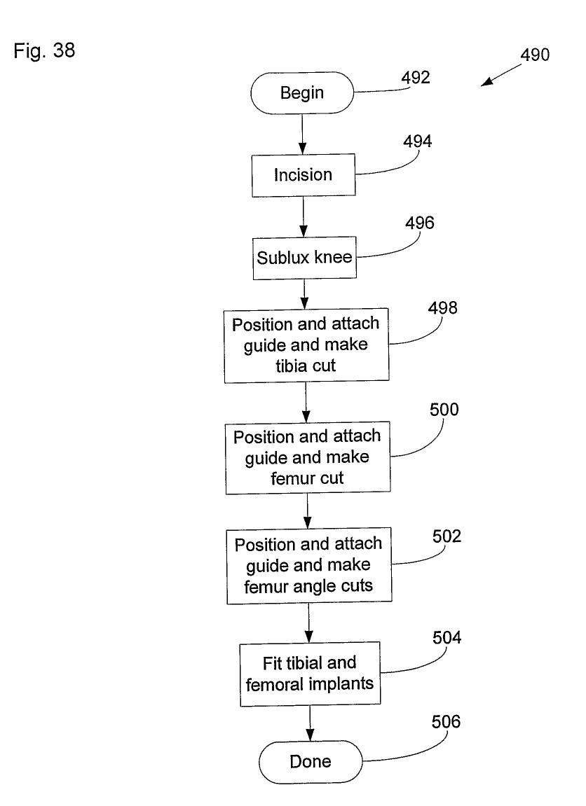

FIG. 38 shows a flow chart illustrating a computer aided surgery part of the method of FIG. 34 for implanting the implant shown in FIGS. 33A-33C according to an embodiment of the invention;

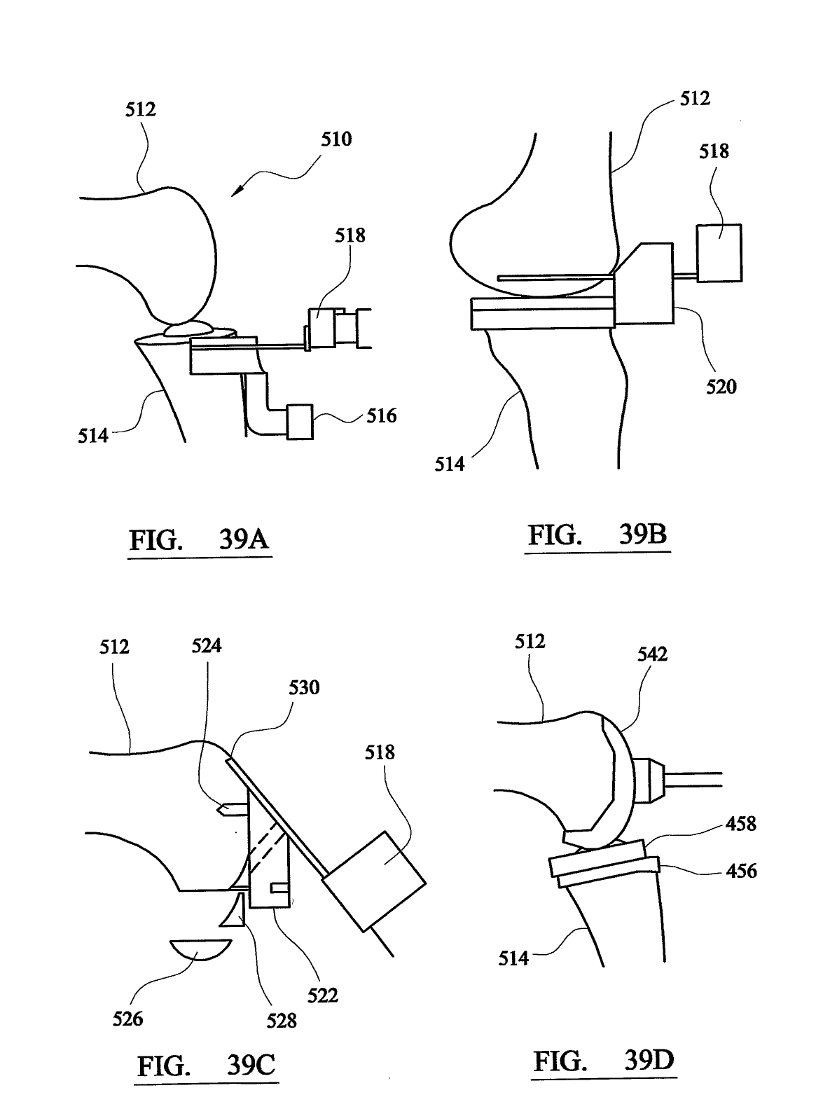

FIGS. 39A to 39D show pictorial representations of a knee having an implant fitted and illustrating parts of the method of FIG. 38;

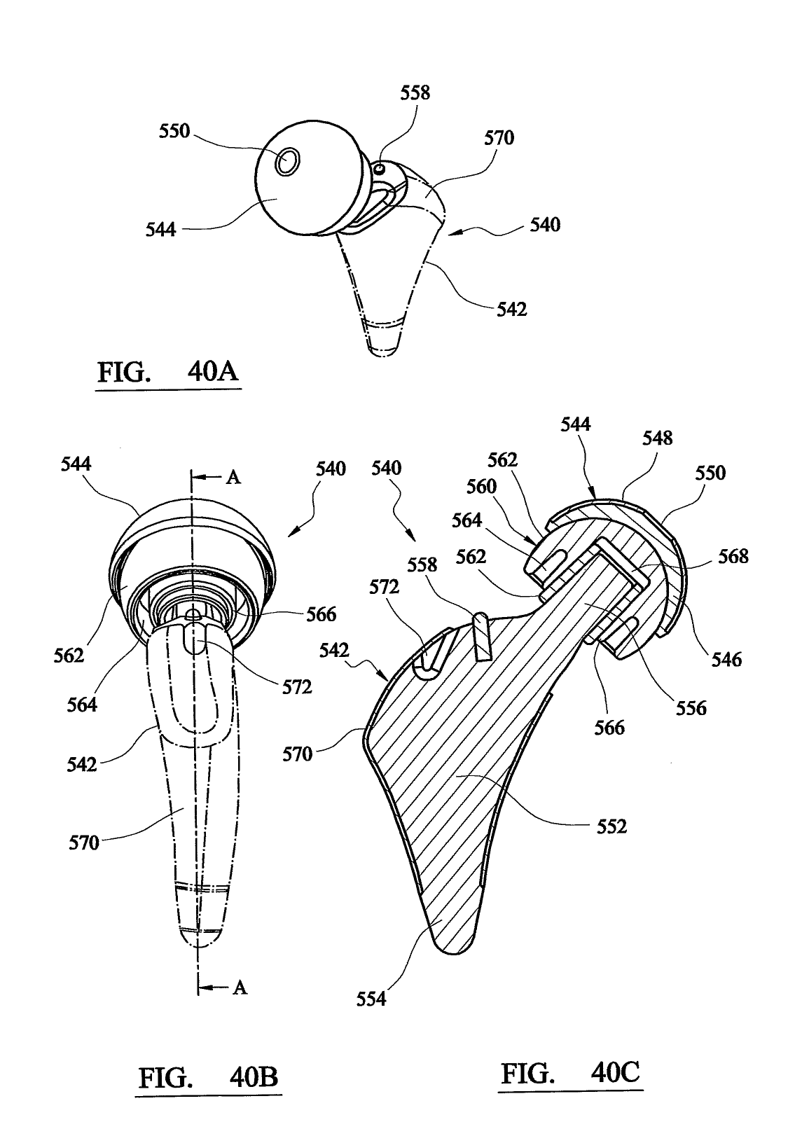

FIGS. 40A, 40B and 40C respectively show a perspective, an end and a cross sectional view along line AA of FIG. 40B of a navigable prosthetic hip implant according to an embodiment of the invention;

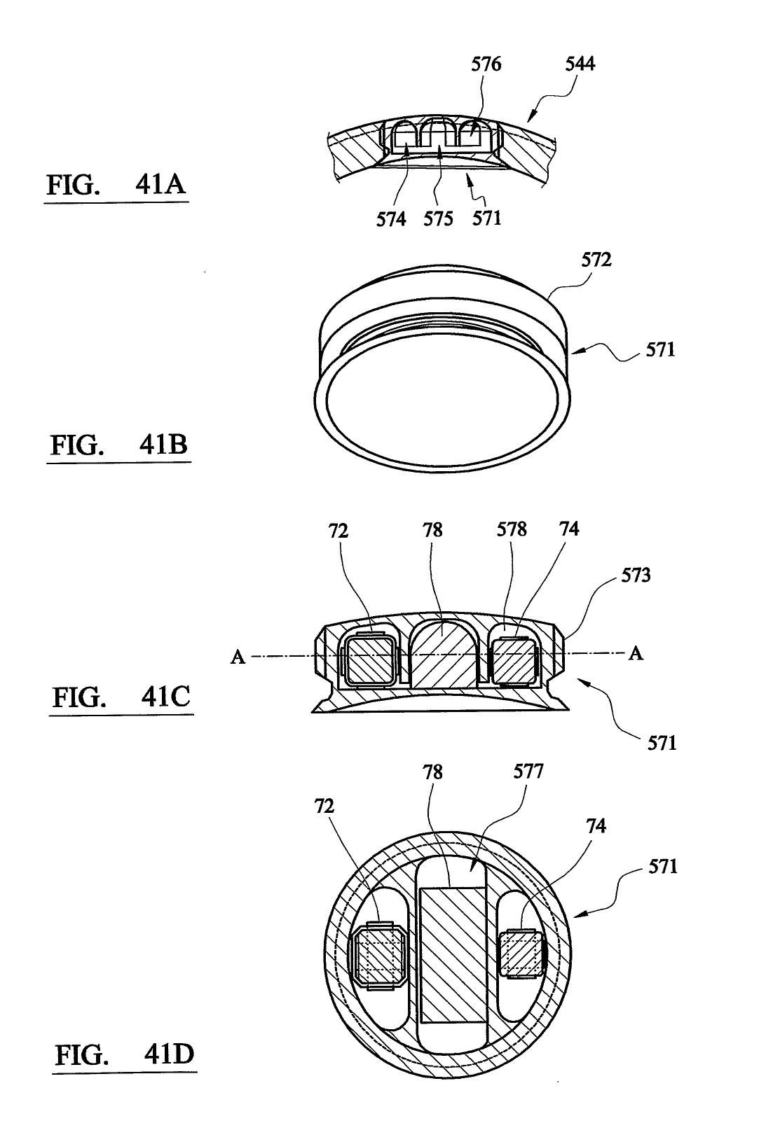

FIGS. 41A, 41B, 41C and 41D respectively show a cross sectional, perspective, longitudinal cross sectional and a transverse cross sectional view along line AA of FIG. 41C of an acetabular implant marker part of the acetabular implant part of the prosthetic hip shown in FIGS. 40A-40C;

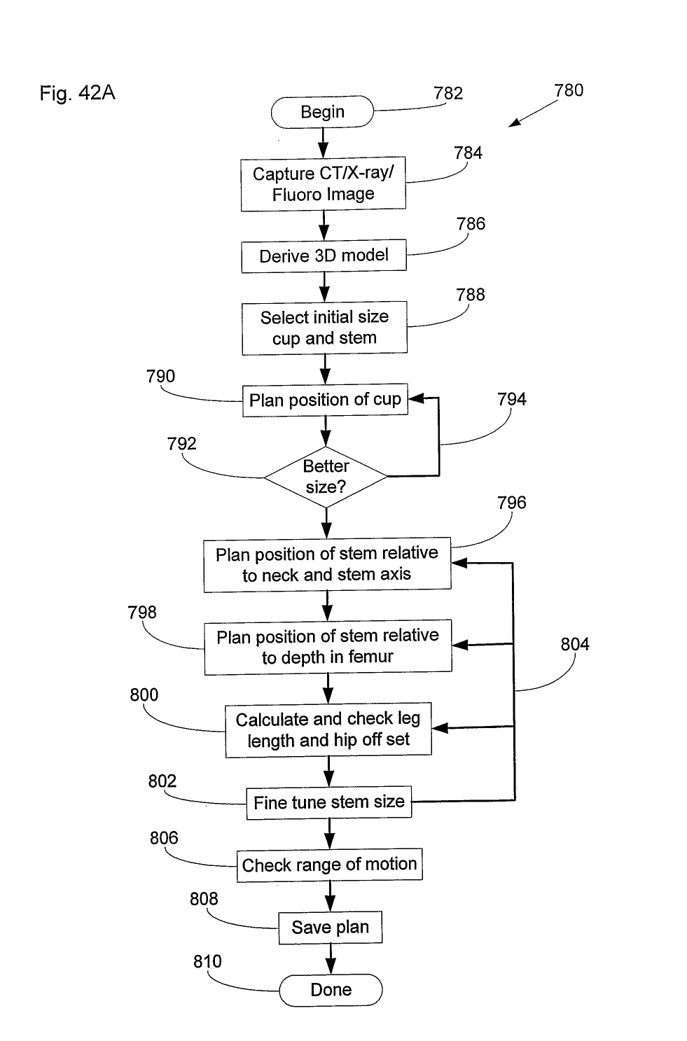

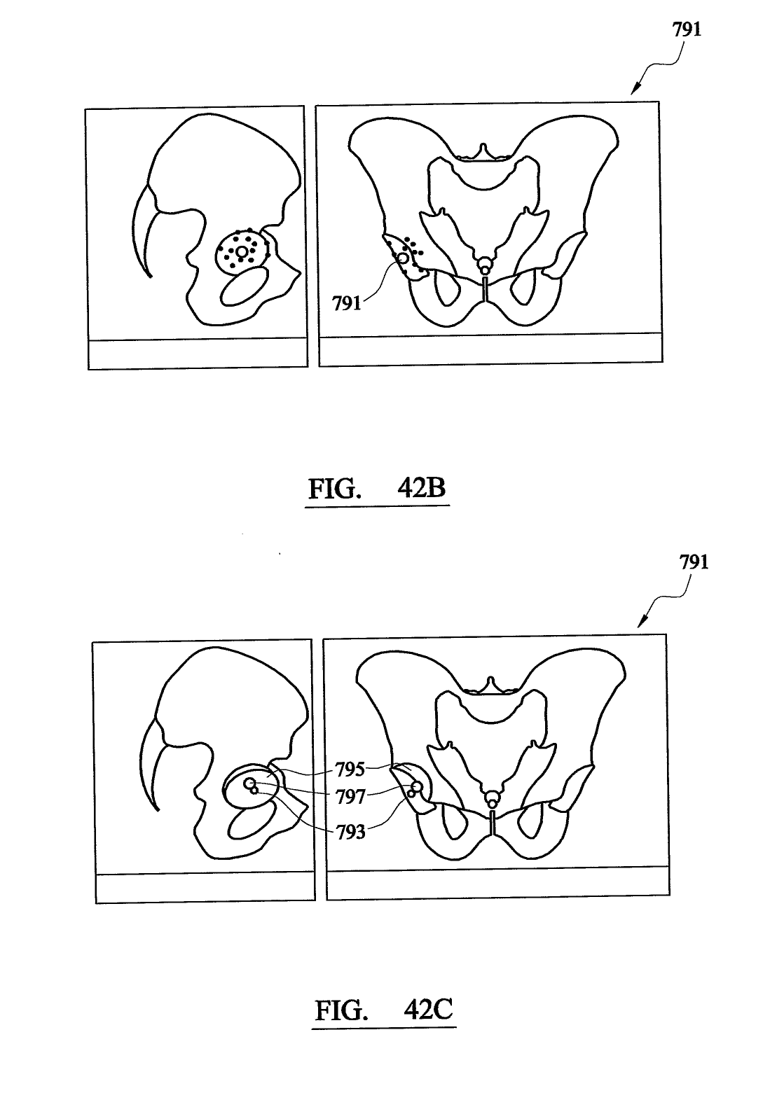

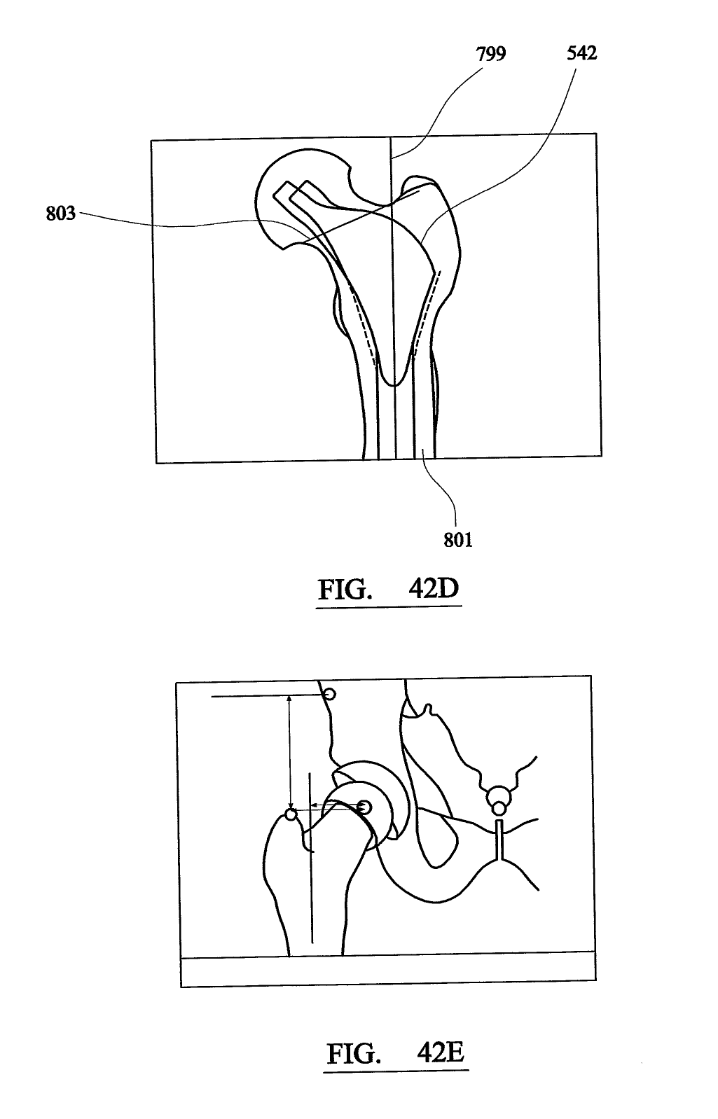

FIG. 42A shows a flow chart illustrating a planning part of a computer aided surgical method for carrying out a hip replacement operation using the orthopaedic operating room according to an embodiment of the invention;

FIGS. 42B to 42E show respective pictorial representations of various steps of the method of FIG. 42A;

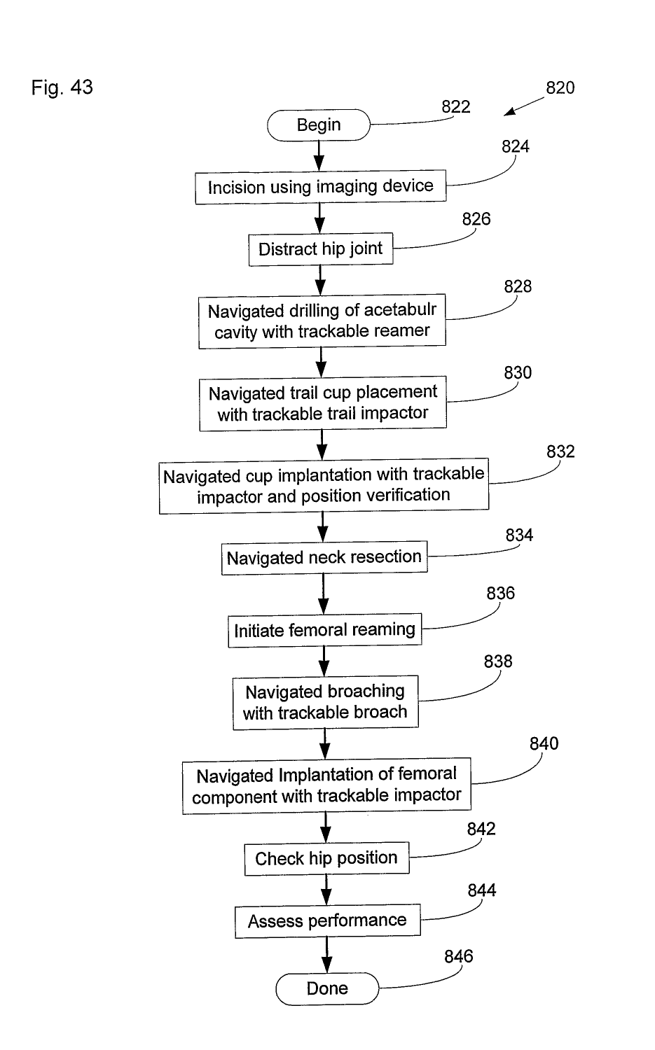

FIG. 43 shows a flow chart illustrating a computer aided surgery method for implanting a navigable prosthetic hip being part of the overall computer aided surgical method for carrying out a hip replacement operation according to an embodiment of the invention;

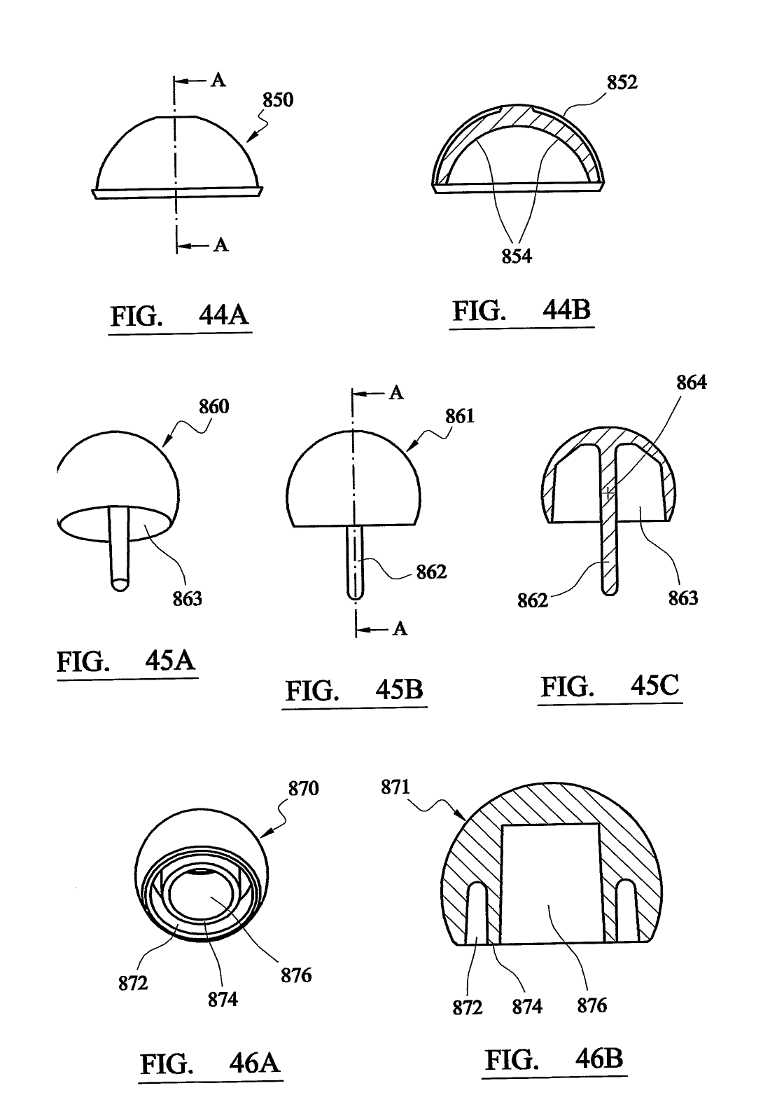

FIGS. 44A and 44B respectively show a side view and a cross sectional view of a further acetabular implant according to an aspect of the invention;

FIGS. 45A, 45B and 45C respectively show perspective, side and cross sectional views of a femoral head implant according to an aspect of the invention;

FIGS. 46A and 46B respectively show perspective and cross sectional views of a further femoral head implant according to an aspect of the invention;

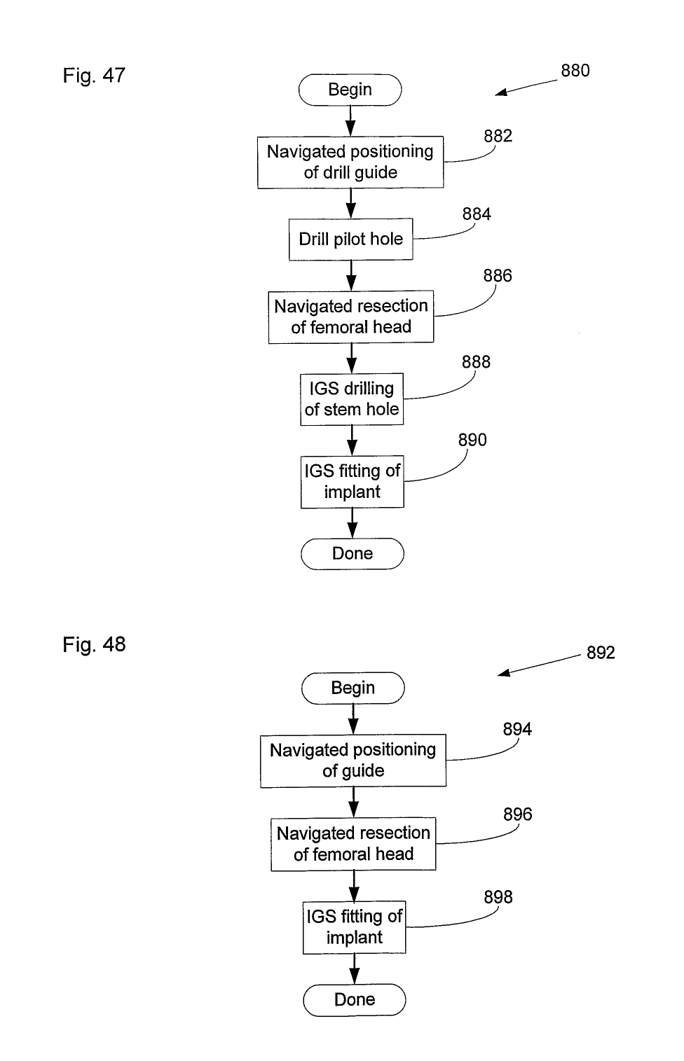

FIG. 47 shows a flow chart illustrating a computer aided surgery part of a further method for fitting the femoral head implant shown in FIGS. 45A-45C according to an embodiment of the invention;

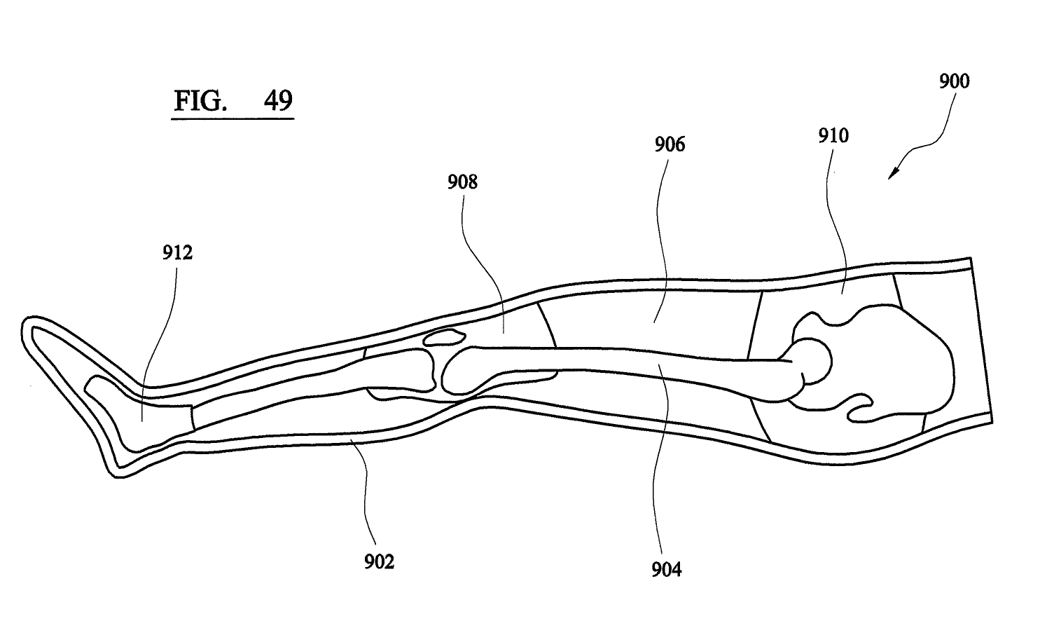

FIG. 48 shows a flow chart illustrating a computer aided surgery part of a further method for fitting the femoral head implant shown in FIGS. 46A and 46B according to an embodiment of the invention;

FIG. 49 shows a cross sectional view through a surgical teaching and training device according to an aspect of the invention and useable in the orthopaedic operating room; and

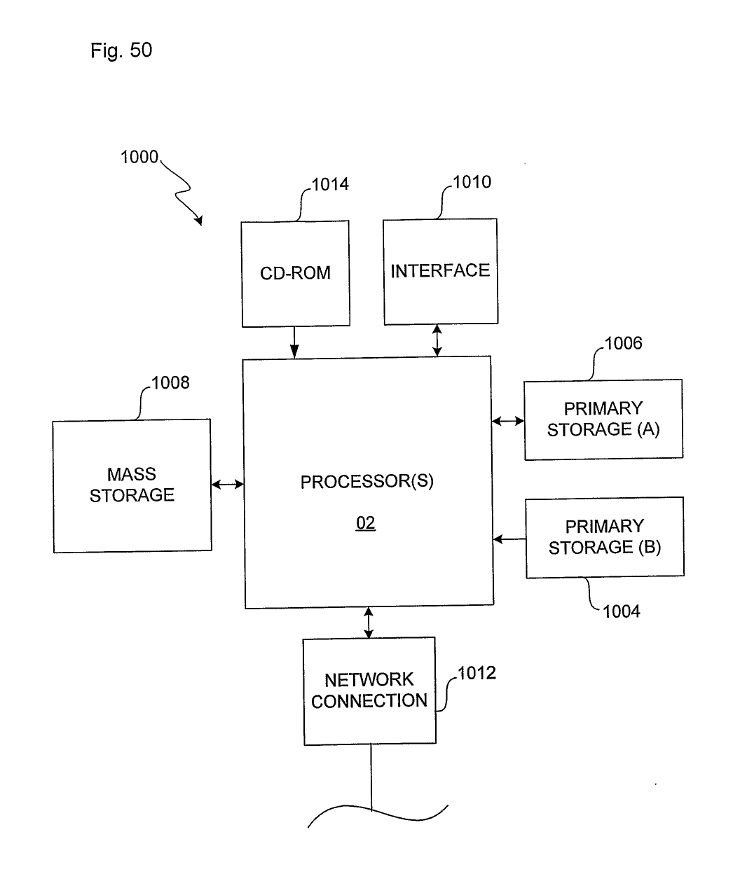

FIG. 50 shows a schematic diagram of a computer control part or parts of the orthopaedic operating room.

Similar items in different Figures generally have common reference numerals unless indicated other wise.

With reference to FIG. 1 there is shown a schematic block diagram of an integrated orthopaedic surgery system 1 which can be used to provide an orthopaedic operating room which itself provides an orthopaedic operating environment within which a surgeon or other medical practitioner can carry out a computer aided orthopaedic surgical procedure. FIG. 1 illustrates some of the major parts and sub-systems of the orthopaedic operating system 1 at a conceptual rather than physical level. That is, FIG. 1 illustrates the functionalities provided by the various parts and sub-systems of the overall system 1 and should not be construed as limiting the actual physical implementation of the functionalities illustrated in FIG. 1.

The orthopaedic operating system 1 includes an operating table 2 which acts as a patient support and on which a patient, or subject, on which an orthopaedic procedure is to be carried out can be located. Various embodiments of patient support 2 will be described in greater detail below with particular reference to FIGS. 14A to 19.

The system 1 also includes a first tracking system 3 and in other embodiments can also include a second tracking system 4. In one embodiment, the first tracking system 3 is a wireless, magnetic tracking system which can track the positions of sensors and provide an indication of the position and orientation of the magnetic sensors, also referred to herein as markers, within a working volume of the tracking system 3. The tracking system 3 has a reference frame, or co-ordinate frame, associated with it and which is also associated with the overall orthopaedic surgery system 1. The tracking system and markers will be described in greater detail below also.

A second tracking system 4 can also be provided and can be a wireless or wire line based tracking system. In one embodiment, the second tracking system can be based on detecting reflected or transmitted infrared radiation. A suitable infrared based tracking system is a suitably configured Vector Vision or Vector Vision 2 system as provided by BrainLab AG of Heimstetten, Germany. The infrared tracking system can include IR camera parts provided by Northern Digital Inc of Canada under the trade name Polaris. This system can also uses active tools or instruments which omit infrared radiation rather than merely reflecting infrared radiation.

The provision of two separate tracking systems allows greater flexibility in the surgeon's work procedures and allows differently marked tools, instruments, implants and reference arrays to be used in order to allow the position of various elements within the system to be determined. It will be appreciated that the infrared based tracking systems require a line of sight to be maintained between the tracked element and infrared detectors and therefore the magnetic field based tracking technology can be preferred as the surgeon does not need to be as mindful of maintaining the line of sight.

The orthopaedic operating system 1 also includes an X-ray or X-ray fluoroscopy based imaging sub-system 5 which can be used to capture images of the patient on the operating table 2 to either pre, intra or post-operatively. In one embodiment, the X-ray imaging system provides a part of an auto-registration feature of the orthopaedic operating system 1 as will be described in greater detail below. The X-ray imaging system can be an X-ray system or can be an X-ray fluoroscopy system.

A real time video imaging system 6 is also provided in the form of a surgical light with an integrated video camera 6. This system can be used to provide illumination of the surgical site and to provide wide filed of view or close up video images.

A surgical site display device 7 is also provided which can be used by the surgeon to display a real time image of the surgical site and on to which other images can be displayed and/or overlayed on the surgical site image. For example, an indication of the location of an incision, cut, an implant, a planned position or an instrument can be displayed as part of an image guided surgical process which will be described in greater detail below.

A large scale display 8 is also provided in the form of a video or image wall. The image display wall has a plurality of imaging regions on which various different images from various different image sources can be displayed in order to provide an immersive environment in which the surgeon can operate and to provide various sources of information to the surgeon in different formats on which to base his surgical activities and decisions. A suitable display wall is available from Barco N.V., based in Belgium.

A control system 9 is also provided which controls and integrates the overall functionality of the various parts and sub-systems of the orthopaedic operating system 1 so that the functionalities are integrated together rather than being disparate parts. The surgeon can control the operation of the operating room via a surgeon interface 10. The surgeon interface 10 can comprise a variety of input and output devices for entering instructions and commands and displaying information to the surgeon. The control system 9 is illustrated schematically by a single suitably programmed general purpose computer device. However in practice, the control system can be implemented by a number of devices so that the control function is distributed throughout the orthopaedic operating system 1. The invention should therefore not be considered to be limited to an implementation involving a single computer and indeed, as will be apparent from the following description, a number of interacting computing devices can be provided.

The surgeon interface 10 can include a gyromouse 11 which is an orientation sensitive input device whereby the surgeon can enter commands to the control system by moving the gyromouse 11 and/or changing its orientation and/or pressing buttons. In this way, the surgeon can control menus and move cursors in order to make selections and enter commands via a graphical user interface displayed on control system display unit 12. A suitable gyromouse is provided by Gyration, Inc of the USA.

Display unit 12 provides a user interface to the surgeon and also displays any number of a plurality of images to the surgeon and is a primary source of information and images available to the surgeon. Display unit 12 has a touch sensitive screen so that the surgeon can enter commands and select options via the screen of display device 12. A plurality of display devices like display 12 can be provided, one for each of the tracking systems, or alternatively a single display device can be used to control and display images from both the tracking systems. The latter option is preferred so as to minimise the number of components in the system.

The surgeon interface 10 can also include a heads up display unit 13 wearable by the surgeon and on which various images, combinations of images and overlays of images can be provided so as to further enhance the surgeon's immersion in the orthopaedic operating environment. A suitable heads up display is provided under the name MOSIS. Another suitable heads up display is the MD-06 as provided by MicroOptical Corporation.

As schematically illustrated in FIG. 1, control system 9 is also in communication with various stored data items and entities which are retrievable from data storage device 14. These data items and entities may be stored locally in the operating room or may be stored remotely and accessed via a network which itself may be wired or wireless. Various data items and entities can be provided in data store 14, such as patient information, patient records, images of patients' scans, models of body parts, instruments, implants, graphics files, workflow programs, orthopaedic surgical planning programs and image guided surgery programs all of which can be provided to control system 9 as required.

As also illustrated in FIG. 1, a communication system or bus 15 is provided over which data and control commands and instructions can be transmitted between the parts of the system so as to integrate the various functional parts of the orthopaedic operating system 1. While various data processing and control operations may be provided locally at the different parts of the system, all of the parts of the system are functionally integrated so that the surgeon can control and configure his working environment so as to optimise it for any specific procedure or any stage of a specific surgical procedure. The nature of the integration of the functionalities of the parts of the system will become apparent from the following description.

FIG. 2 shows a perspective view of a simulation of an orthopaedic operating room in which the orthopaedic operating system 1 has been integrated. FIG. 3 shows a plan view of the operating room illustrated in FIG. 2. As illustrated in FIG. 2, the image wall 8 can display a plurality of separate images in different regions, which images can be selected for display by the surgeon via the surgeon interface 10. The image wall 8 can be controlled to display a single large image or multiple images of different sizes depending on the surgeon's requirements. Various different images can be displayed on video wall 8, including patient scan images, such as X-rays, CT, fluoroscopy and ultrasound scan images, still or animated real time images of the surgical site, such as video images captured by camera and lighting system 6, images of models of patient body parts, images of real and virtual implants and instruments, images generated by surgical planning software and images generated by image guided surgery programs so as to guide the positioning of instruments and implants during a surgical procedure.

As also illustrated in FIGS. 2 and 3, the surgical lighting camera system 6, control system display device 12, X-ray detector parts 16, 16′ and an orbiter 18′ are all suspended from the ceiling of the operating room so as to provide a clear floor area around the operating table 2. Further, X-ray sources 17, 17′ of the X-ray imaging system 5 are integrated into or under the floor.

With reference to FIG. 4 there is shown a schematic block diagram of an image handling and control sub-system part 190 of the orthopaedic operating system 1. Image handling sub-system 190 is based around a video mixing and control system 191 which receives as input various images from various sources provided by various imaging parts of the system. The video mixing and control system, under control of a multimedia computer system 192 handles the formatting and direction of the images from the various sources and sends them to the appropriate display devices throughout the orthopaedic operating system 1.

The video mixing and control system 191 receives images of various tracked elements of the system from the magnetic tracking system 3 and/or the infrared tracking system 4. A video camera part 18 of the surgical lighting and camera system 6 provides a real time video input. Data store 14 can provide stored patient scan images and images of models of bones, instruments, implants and virtual representations of other parts of the orthopaedic operating system. An endoscope 193 can also be provided which acts as a further source of images which can be displayed. Any other sources of video 194 can also be supplied to the video mixing and control system. An image capturing part of the surgical site display device 7 can also provide an input to the video mixing and control system and can also receive images to overlay on a surgical site image, displayed to the surgeon.

The video mixing and control system also outputs images for display on the heads up display unit 13, for display on the different regions of the image wall 8 and also for display on the control system monitor 12. Control system monitor 12 is a touch screen device as indicated previously via which the surgeon can enter commands which are processed by video control system 195 in order to control or vary the sources of images to be displayed, the nature of the images to be displayed and the display devices on which the images are to be displayed. Examples of the types of images that can be displayed will become apparent from the following description.

With reference to FIG. 5 there is shown a flowchart illustrating at a high level a general method 600 of use of the orthopaedic operating system 1. The method 600 includes three general stages. The first stage 602 includes pre-operative procedures which can include capturing various images of the patient's body part, such as CT scans, ultrasound scans, X-ray scans and/or X-ray fluoroscopy images. Various other pre-operative operations can be carried out, such as an assessment of the orthopaedic performance of the patient so as to determine the appropriate surgical orthopaedic treatment.

Also during the pre-operative phase 602, markers detectable and trackable by the wireless magnetic tracking system 3 can be percutaneously implanted in the bones of the patient. The positions of the patient's bones can then be tracked so as to aid in the assessment of the orthopaedic performance of the patient. Also during the pre-operative phase 602, the planning of the orthopaedic surgical procedure can be carried out using a surgical planning software application. In some embodiments of the method, patient registration is carried out pre-operatively. A number of these operations can be carried out in the operating room or alternatively prior to the patient entering the operating room.

A second phase 604 corresponds to intra-operative preparations, that is, generally, operations between the beginning of surgery, i.e. the initial incision, and the end of surgery, i.e. closing the surgical wound. Intra-operative procedures can include the registration of the patient's body parts, in some embodiments, intra-operative surgical planning of the positions of implants, navigated and image guided surgical steps, including the preparation of bones and placement of implants, and immediate assessment of the orthopaedic performance of the implanted orthopaedic implants. It is also possible to capture images of the patient's body parts intra-operatively and use those images in the image guided or navigated surgical steps.

A third phase of the overall method 600 includes post-operative procedures, which can include an assessment of the orthopaedic performance of the patient, including viewing images of the kinematic performance of implanted orthopaedic implants, capturing images of the patient's body parts and implants and removing implanted bone markers. Some or all of these operations can be carried out in the operating room or subsequently in other medical facilities.

With reference to FIG. 6 there is shown a schematic block diagram of a software architecture 610 illustrating the major functional components used in the orthopaedic operating system 1. The software architecture is intended to be conceptual only and the individual blocks as merely to clarify the nature of the processes carried out and are not intended to limit the invention to the specific software architecture illustrated. Rather, a number of the functions will be distributed between different programs and execution of those programs will be distributed throughout various parts of the orthopaedic operating system 1.

As illustrated in FIG. 6, software architecture 610 includes a tracking module 612 which receives sets of identifier and positional data items 611 from each of the markers tracked by the tracking systems. The tracking module 612 continuously supplies an indication of the position and orientation of the element, e.g. bone, instruments, implants, associated with each of the tracked markers to a planning module 614 and a computer aided surgery module 616, and any other software component that needs access to the position of the trackable elements of the system. The planning module 614 includes a number of routines which can be used to plan the size and position of various orthopaedic implants so as to appropriately construct the joint of a patient. The computer aided surgery module 616 provides various procedures and routines by which surgical instruments and implants can be navigated and displays images which allow image guided surgical procedures to be carried out by on the surgical plan created using the planning module.

A general workflow module 618 provides a definition of the various steps to be carried out by the surgeon in planning and executing a particular surgical operation e.g. the steps involved in a hip replacement or knee replacement operation, and generally controls the overall process of registering the patient, planning the procedure and executing the procedure, as schematically illustrated by arrow 620.

A patient registration module 620 provides various routines and procedures allowing images of patient body parts and virtual images of various elements used in the orthopaedic operating room, e.g. body parts, instruments and implants, to be registered with the actual position of the elements in the reference frame of the orthopaedic operating room system. Various registration procedures can be used depending on the nature of the registration procedure to be used, e.g. captured patient image based or captured patient image free, and whether it is a pre-operative or intra-operative registration procedure. For example registration information may be required by the planning module 614 and/or by the computer aided surgery module 616 if an intra-operative registration procedure is used.

An image processing and handling module 622 is also provided and interacts with the planning and orthopaedic surgery modules to provide image handling, processing and display services. The image processing module has access to the data store 14 which includes patient body scan image data 624 and stored images 626 of various elements, and models of the elements, used and tracked in the orthopaedic operating room, such as generic bone shapes, instruments and implants. Using the stored image data, real time 3D representations of the patient's body parts, implants and instruments can be displayed in real time both during the planning and computer aided surgery stages of the overall method.

With reference to FIG. 7, there is shown a perspective view of parts of the X-ray imaging system 5. The X-ray imaging system includes a first X-ray source 17 located in the floor of the operating room and a second X-ray source 17′ also in the floor of the operating room. A first X-ray or X-ray fluoroscopy detector 16 is provided suspended from the roof of the operating room and is associated with the first X-ray source. A second X-ray or X-ray fluoroscopy detector 16′ is also suspended from the ceiling of the operating room and is associated with the second X-ray source 17′. The X-ray sources and X-ray detectors are positioned relative to the operating table 2 so as to be able to capture a first image from a first direction through the patient's body and a second image through a second direction through the patient's body. It is preferred if these images are taken in directions approximately 90^{B} apart. The captured X-ray images can be used subsequently as part of an automatic registration procedure as will be described in greater detail below. Alternatively, the X-ray imaging system can merely be used in order to generate pre, intra or post-operative X-ray images of the body parts of the patient.

Further the position of the X-ray sources and detectors are known to the tracking and navigation systems. Bone markers implanted in the patient that also show on the X-rays, can provide one mechanism by which the patient anatomy can automatically be registered by the navigation system. The X-ray system can be controlled by the surgeon via the surgeon interface and the acquired image can be displayed on the image wall 8 screens.

In one embodiment no preoperative scan or X-ray is taken of the patient and instead 2D fluoroscopy images are captured using the X-ray based imaging system and from these a 3D model of the patient's bones is built. Orthogonal X-ray shots are taken and the X-ray image data is used to morph a generic 3D model of the bone to customise the model for that specific patient. With this digital model all aspects of the optimal implant position can be planned virtually, e.g. component size, leg length, offset, stem anteversion and cup position for a hip implant. There are a number of advantages to this approach. This technique is particularly useful in revision arthroplasty when a CT scan would not be possible but when an accurate 3 dimensional model will enable restoring joint anatomy even when significant bone erosion has occurred and landmarks have been destroyed. The technique can also be used for trauma and spinal applications.

With reference to FIG. 8 there is shown a schematic view of the surgical site imaging and display device 7. The surgical site imaging and display device 7 includes an image capturing device 630 in the form of a video camera having a field of view schematically indicated by dashed lines 632. A display part 634 of the device includes a display element 636 in the form of a TFT display. Electronic control circuitry 638 is also provided which interfaces between the image capturing device 630 and display device 636. The upper display part 634 of the surgical site display device 7 is mounted on a support 640 attached to a base 642 having wheels or casters 644. A marker 646, trackable by a one of the tracking systems is also attached to the surgical site display device so that the position of the display device within the reference frame of the operating room can be determined.

In use, the surgical site display device is positioned with the field of view 632 covering the surgical site of the patient, e.g. the knee or hip. The current image of the image capturing device 630 is displayed in the display part 636 so that the surgeon can see the patient's body immediately below the surgical site display device and in registration with the surgical site.

The surgeon can then select to display in place of the image of the patient's body, or overlay on the display of the patient's body, visual representations of useful information, such as the planned position of an initial incision, the planned or navigated positions of instruments or tools, such as drill guides, and the planned positions of implants, and three dimensional images of the implants and body parts, e.g. the patient's bones. Also, scan images or images derived from patient's scans can also be displayed in the display screen 636, such as X-ray images, CT scan images or ultrasound images. Hence, the surgeon can concurrently display various visual forms of information concurrent with a current display of the surgical site of the patient.

There will now be described an embodiment of the wireless, magnetic based tracking system 3, various embodiments of operating room table 2, and various embodiments of wirelessly magnetically detectable and trackable markers for implanting in the bones of patients, for use with orthopaedic implants and for use with instruments and tools.

Unless the context indicates otherwise, in the following the terms “marker” and “sensor” or “position sensor” will be used interchangeably to refer to a device trackable by the tracking system, the position and/or orientation of which can be determined. An “implantable marker” will generally be used to refer to a marker that has been adapted so as to be implanted within the bone of a patient. The terms “implant”, “orthopaedic implant”, “prosthesis” or “prosthetic implant”, or variations thereof will generally be used to refer to a prosthetic orthopaedic implant for implanting in a body to replace a part of a joint or bone. Such an implant can bear or otherwise have a marker or sensor attached thereto, or therein, so as to provide a marked implant trackable by the tracking system.

FIG. 9 is a schematic, pictorial illustration of a magnetic tracking system 3 part of the orthopaedic operating system for use in computer aided surgery. In the pictured embodiment, a surgeon 22 is preparing to perform a procedure on a leg 24 of a patient 26. The surgeon uses a tool 28 to implant an implantable marker 30 in the form of a specially adapted bone screw in leg 24. Both the tool and the screw contain miniature, wireless markers or position sensors, which are described in detail hereinbelow. The bone screw provides a housing within which the wireless marker is hermetically sealed. Each sensor generates and transmits signals that are indicative of its location and orientation coordinates, in response to an external magnetic field produced by a set of field generator coils 32 (also referred to as radiator coils). Typically, multiple implantable markers, in the form of a screw with a position sensor therein, are implanted by surgeon 22 at key locations in the patient's bone.

Additionally or alternatively, position sensors or markers may be fixed to implants, such as a prosthetic joint or intramedullary insert, in order to permit the position of the implant to be monitored, as well. For example, the use of such position sensors in a hip implant is shown U.S. patent application Ser. No. 10/029,473.

Field generator coils 32 are driven by driver circuits 34 to generate electromagnetic fields at different, respective sets of frequencies {T₁}, {T₂} and {T₃}. Typically, the sets comprise frequencies in the approximate range of 100 Hz-30 kHz, although higher and lower frequencies may also be used. The sets of frequencies at which the coils radiate are set by a computer 36, which serves as the system controller for system 20. The respective sets of frequencies may all include the same frequencies, or they may include different frequencies. In any case, computer 36 controls circuits 34 according to a known multiplexing pattern, which provides that at any point in time, no more than one field generator coil is radiating at any given frequency. Typically, each driver circuit is controlled to scan cyclically over time through the frequencies in its respective set. Alternatively, each driver circuit may drive the respective coil 32 to radiate at multiple frequencies simultaneously.

For the purposes of tracking system 3, coils 32 may be arranged in any convenient position and orientation, so long as they are fixed in respect to some reference frame, and so long as they are non-overlapping, that is, there are no two field generator coils with the exact, identical location and orientation. Typically, for surgical applications such as that shown in the figures, coils 32 comprise wound annular coils about 15-20 cm in outer diameter (O.D.) and about 1-2 cm thick, in a triangular arrangement, wherein the centers of the coils are about 80-100 cm apart. The coil axes may be parallel, as shown in this figure, or they may alternatively be inclined, as shown, for example, in FIGS. 14A and 14B. Bar-shaped transmitters or even triangular or square-shaped coils could also be useful for such applications.

In orthopaedic and other surgical applications, it is desirable that coils 32 be positioned away from the surgical field, so as not to interfere with the surgeon's freedom of movement. On the other hand, the coils should be positioned so that the working volume of the tracking system includes the entire area in which the surgeon is operating. At the same time, the locations and orientations of coils 32 should be known relative to a given reference frame in order to permit the coordinates of tool 28 and implantable marker 30 to be determined in that reference frame.

In order to meet these potentially-conflicting requirements, coils 32 are mounted on a reference structure 40. In the embodiment of FIG. 9, structure 40 comprises multiple arms 42, which are fixed to an articulated base 44. Alternative reference structures and configurations are shown in the figures that follow. Arms 42 hold coils 32 in known relative positions. Base 44, however, is capable of tilting, turning and changing the elevations of arms 42, so as to enable surgeon 22 to position coils 32 in convenient locations. The movement of base 44 may be controlled by computer 36, so that the computer is also aware of the actual locations of coils 32.

Alternatively or additionally, an image registration procedure may be used to calibrate the positions of coils 32 relative to patient 26. An exemplary registration procedure, based on X-ray imaging, is described in U.S. Pat. No. 6,314,310 whose disclosure is incorporated herein by reference. Further alternatively or additionally, a reference sensor, fixed to patient 26 or to the operating table in a known location, may be used for calibration. The use of reference sensors for this purpose is described, for example, in U.S. Pat. No. 5,391,199.

The position sensors in implantable marker 30 and tool 28 typically comprise sensor coils, in which electrical currents are induced to flow in response to the magnetic fields produced by field generator coils 32. An exemplary arrangement of the sensor coils is shown in FIG. 11A below. The sensor coils may be wound on either air cores or cores of magnetic material. Typically, each position sensor comprises three sensor coils, having mutually orthogonal axes, one of which is conveniently aligned with the longitudinal axis of tool 28 or of the screw housing. The three coils may be concentrically wound on a single core, or alternatively, the coils may be non-concentrically wound on separate cores, and spaced along the longitudinal axis of the tool or screw housing. The use of non-concentric coils is described, for example, in the above-mentioned PCT Patent Publication WO 96/05768 and in the corresponding U.S. patent application Ser. No. 09/414,875. Alternatively, the position sensors may each comprise only a single sensor coil or two sensor coils. Further alternatively, screw housing and tool 28 may include magnetic position sensors based on sensing elements of other types known in the art, such as Hall effect sensors.

At any instant in time, the currents induced in the sensor coils comprise components at the specific frequencies in sets {T₁}, {T₂} and {T₃} generated by field generator coils 32. The respective amplitudes of these currents (or alternatively, of time-varying voltages that may be measured across the sensor coils) are dependent on the location and orientation of the position sensor relative to the locations and orientations of the field generator coils. In response to the induced currents or voltages, signal processing and transmitter circuits in each position sensor generate and transmit signals that are indicative of the location and orientation of the sensor. These signals are received by a receiving antenna (shown, for example, in FIG. 14A), which is coupled to computer 36. The computer processes the received signals, together with a representation of the signals used to drive field generator coils 32, in order to calculate location and orientation coordinates of implantable marker 30 and tool 28. The coordinates are used by the computer in driving display 12, which shows the relative locations and orientations of the tool, screw and other elements (such as prosthetic implants) to which markers or position sensors have been fixed.

Circuitry 78 also stores a unique identifier for marker 70 and the unique identifier is also transmitted to the tracking system, so that the tracking system can determine the identity of the marker from which positional data is being received. Hence the tracking system can discriminate between different markers when multiple markers are present in the working volume of the tracking system.

Although in FIG. 9, system 20 is shown as comprising three field generator coils 32, in other embodiments of the present invention, different numbers, types and configurations of field generators and sensors may used. A fixed frame of reference may be established, for example, using only two non-overlapping field generator coils to generate distinguishable magnetic fields. Two non-parallel sensor coils may be used to measure the magnetic field flux due to the field generator coils, in order to determine six location and orientation coordinates (X, Y, Z directions and pitch, yaw and roll orientations) of the sensor. Using three field generator coils and three sensor coils, however, tends to improve the accuracy and reliability of the position measurement.

Alternatively, if only a single sensor coil is used, computer 36 can still determine five position and orientation coordinates (X, Y, Z directions and pitch and yaw orientations). Specific features and functions of a single coil system (also referred to as a single axis system) are described in U.S. Pat. No. 6,484,118, whose disclosure is incorporated herein by reference.

When a metal or other magnetically-responsive article is brought into the vicinity of an object being tracked, such as implantable marker 30 or tool 28, the magnetic fields in this vicinity are distorted. In the surgical environment shown in FIG. 9, for example, there can be a substantial amount of conductive and permeable material, including basic and ancillary equipment (operating tables, carts, movable lamps, etc.), as well as invasive surgery apparatus (scalpels, scissors, etc., including tool 28 itself). The magnetic fields produced by field generator coils 32 may generate eddy currents in such articles, and the eddy currents then cause a parasitic magnetic field to be radiated. Such parasitic fields and other types of distortion can lead to errors in determining the position of the object being tracked.

In order to alleviate this problem, the elements of tracking system 3 and other articles used in the vicinity of the tracking system are typically made of non-metallic materials when possible, or of metallic materials with low permeability and conductivity. For example, reference structure 40 may be constructed using plastic or non-magnetic composite materials, as may other articles in this vicinity, such as the operating table. In addition, computer 36 may be programmed to detect and compensate for the effects of metal objects in the vicinity of the surgical site. Exemplary methods for such detection and compensation are described in U.S. Pat. Nos. 6,147,480 and 6,373,240, as well as in U.S. patent application Ser. Nos. 10/448,289, filed May 29, 2003 and 10/632,217, filed Jul. 31, 2003, all of whose disclosures are incorporated herein by reference.

FIG. 10A is a schematic, sectional illustration showing implantation of implantable marker 30 into a bone 50, such as the femur of patient 26, in accordance with an embodiment of the present invention. To insert this embodiment of the implantable marker 30, surgeon 22 can make an incision through overlying soft tissue 52, and then rotates the screw into bone 50 using tool 28, for example. Note that in this embodiment, implantable marker 30 has no wired connection to elements outside the body. Further, the sensor or marker within the housing is actually located within the bone of the patient and is not merely attached to the bone by a support structure. Typically, implantable marker 30 is between 5 and 15 mm long, and is about 2-4 mm in diameter. To avoid interfering with reception and transmission of signals by the sensor that it contains, screw housing typically comprises a non-magnetic material, which may comprise metals, alloys, ceramics, plastics or a combination of such materials. The configuration and operation of the circuits in implantable marker 30 are described hereinbelow with reference to FIGS. 11A and 11B.

FIG. 10B is a schematic, sectional illustration showing another implantable marker or position sensor device 54, in accordance with an alternative embodiment of the present invention. Device 54 comprises a marker in a screw housing, which is coupled by wires 58 to an external unit 60. The screw housing and marker are inserted into bone 50 in substantially the same manner as is implantable marker 30 (leaving wires 58 to pass out of the patient's body through soft tissue 52). In this case, however, because some elements of marker device 54 are contained in external unit 60, the implantable marker part 56 may generally be made smaller than implantable marker 30. For example, screw 56 may be between 5 and 10 mm long, and 2 and 4 mm in diameter. Again, the position sensitive part of the marker is actually located within the bone and not merely connected to the bone by a support. The reduced housing size is helpful in reducing trauma and possible damage to bone 50. Further details of device 54 are shown in FIG. 12.

FIG. 11A is a schematic, pictorial illustration of a marker or wireless position sensor 70 that is contained in screw housing to provide the implantable marker 30, in accordance with an embodiment of the present invention. Sensor 70 in this embodiment comprises three sets of coils: sensor coils 72, power coils 74, and a communication coil 76. Alternatively, the functions of the power and communication coils may be combined, as described in the above-mentioned U.S. patent application Ser. No. 10/029,473. Coils 72, 74 and 76 are coupled to electronic processing circuitry 78, which is mounted on a suitable substrate 80, such as a flexible printed circuit board (PCB). Details of the construction and operation of circuitry 78 are described in U.S. patent application Ser. No. 10/029,473 and in the above-mentioned U.S. patent application Ser. No. 10/706,298, which are incorporated herein by reference.

Although for simplicity, FIG. 11A shows only a single sensor coil 72 and a single power coil 74, in practice sensor 70 typically comprises multiple coils of each type, such as three sensor coils and three power coils. The sensor coils are wound together, in mutually-orthogonal directions, on a sensor core 82, while the power coils are wound together, in mutually-orthogonal directions, on a power core 84. Typically, each of the three power coils comprises about 30-40 turns of wire having a diameter of at least about 40:m, while power core 84 is a ferrite cube of about 1.5-2 mm on a side. Each of the three sensor coils typically comprises between about 700 and 3000 turns of 11:m diameter wire, while sensor core 82 is a ferrite cube of about 1.8-2.4 on a side. (It will be understood that these dimensions are given by way of example, and the dimensions may in practice vary over a considerable range.) Alternatively, the sensor and power coils may be overlapped on the same core, as described, for example in U.S. patent application Ser. No. 10/754,751, filed Jan. 9, 2004, whose disclosure is incorporated herein by reference. It is generally desirable to separate the coils one from another by means of a dielectric layer (or by interleaving the power and sensor coils when a common core is used for both) in order to reduce parasitic capacitance between the coils.

In operation, power coils 74 serve as a power source for sensor 70. The power coils receive energy by inductive coupling from an external driving antenna (shown, for example, in FIG. 14A). Typically, the driving antenna radiates an intense electromagnetic field at a relatively high radio frequency (RF), such as in the range of 13.5 MHz. The driving field causes currents to flow in coils 74, which are rectified in order to power circuitry 78. Meanwhile, field generator coils 32 (FIG. 9) induce time-varying signal voltages to develop across sensor coils 72, as described above. Circuitry 78 senses the signal voltages, and generates output signals in response thereto. The output signals may be either analog or digital in form. Circuitry 78 drives communication coil 76 to transmit the output signals to a receiving antenna (also shown in FIG. 14A) outside the patient's body. Typically, the output signals are transmitted at still higher radio frequencies, such as frequencies in the rage of 43 MHz or 915 MHz, using a frequency-modulation scheme, for example. Additionally or alternatively, coil 76 may be used to receive control signals, such as a clock signal, from a transmitting antenna (not shown) outside the patient's body. Although certain frequency ranges are cited above by way of example, those skilled in the art will appreciate that other frequency ranges may be used for the same purposes.

In another embodiment, not shown in the figures, sensor coils 72 are non-concentric. In this embodiment, each of the sensor coils typically has an inner diameter of about 0.5-1.3 mm and comprises about 2000-3000 turns of 11:m diameter wire, giving an overall coil diameter of 9 mm. The effective capture area of the coil is then about 400 mm². It will be understood that these dimensions are given by way of example only and the actual dimensions may vary over a considerable range. In particular, the size of the sensor coils can be as small as 0.3 mm (with some loss of sensitivity) or as large as 2 mm or more. The wire size of the sensor coils can range from 10-31:m, and the number of turns between 300 and more than 3000, depending on the maximum allowable size and the wire diameter. The effective capture area of the sensor coils is typically made as large as feasible, consistent with the overall size requirements. The sensor coils are typically cylindrical, but other shapes can also be used. For example, barrel-shaped or square coils may be useful, depending on the geometry of the screw housing.

FIG. 11B is a schematic, pictorial illustration of a marker or wireless position sensor 90, in accordance with another embodiment of the present invention. Sensor 90 differs from sensor 70, in that sensor 90 comprises a battery 92 as its power source, instead of power coils 74. In other respects, the operation of sensor 90 is substantially similar to that of sensor 70, as described above. Use of battery 92 has the advantages of supplying higher operating power to circuitry 78, while avoiding the need to irradiate patient 26 with an intense electromagnetic field in order to provide inductive RF power to the sensor. On the other hand, incorporating battery 92 in sensor 90 typically increases the length of the sensor, by comparison to sensor 70, and therefore may require the use of a longer screw housing to contain the sensor. In addition, the operating lifetime of sensor 70 is effectively unlimited, while that of sensor 90 is limited by the lifetime of battery 92. Sensor 90 is particularly suited for marking tools or instruments as the marker is available for replacement of the battery as required.

FIG. 12 is a schematic, pictorial illustration showing details of device 54, in accordance with an embodiment of the present invention. The external features of device 54 and its implantation in bone 50 were described above with reference to FIG. 10B. Device 54 comprises an internal marker or sensing unit 94, which is contained in a housing bearing a screw thread (not shown) to provide the implantable marker part 56. Typically, sensing unit 94 contains only sensor coils 72, and possibly elements of circuitry 78. This arrangement allows the size of the housing and hence the implantable marker to be minimized. External unit 60 typically contains a battery 96 and circuit elements 98, which comprise some or all of circuitry 78, as well as communication coil 76. The battery may thus be replaced when necessary, without removing marker 56 from the bone. On the other hand, whereas sensors 70 and 90 are contained completely enclosed within their housing, and thus leave no elements protruding outside the patient's body, device 54 can operate only when external unit 60 is connected outside the body to wires 58 that communicate with sensing unit 94.

FIG. 13 is a schematic, pictorial illustration showing details of a marked tool or instrument 28, in accordance with an embodiment of the present invention. Tool 28 comprises a handle 100 and a shaft 102. A tool marker or sensor 104 fits snugly into a suitable receptacle inside handle 100. Sensor 104 comprises sensing and communication circuits 106, which are powered by a battery 108. Typically, circuits 106 comprise three sensing coils, a communication coil and processing circuitry, as in sensor 90 (FIG. 11B). The sensing coils are similar to coils 72, and sense the location and orientation of sensor 104 relative to the magnetic fields generated by field generator coils 32 (FIG. 9). The communication coil conveys position signals to computer 36. The operation of circuits 106 is thus similar to that of the circuits in sensors 70 and 90, although elements of circuits 106 may be made larger and consume greater power than the corresponding elements in sensors 70 and 90.

Tool marker or sensor 104 may be permanently housed inside tool 28, or the sensor may alternatively be removable (to replace battery 108, for example). Because the geometry of tool 28 is known, the location and orientation of handle 100, as indicated by sensor 104, indicates precisely the location and orientation of the distal tip of shaft 102. Alternatively, the tool sensor may be miniaturized and may thus be contained inside shaft 102. Optionally, the tool sensor may be calibrated before use in order to enhance the precision with which the shaft position is measured.

FIGS. 14A and 14B are schematic, pictorial illustrations showing insertion of a location pad 110 into an opening in an operating table 112, in accordance with an embodiment of the present invention. Table 112, and other tables described below, are particular embodiments of the table 2 of the operating room. Pad 110 may be used as the reference structure in system 20 (FIG. 9), in place of structure 40. Pad 110 comprises an integral unit, which holds three field generator coils 32 in fixed positions. The field generator coils in this case are angled diagonally inward. In FIG. 14A pad 110 is shown prior to insertion into the table, while in FIG. 14B the pad has been slid into place.

Location pad 110 is also seen in FIG. 14A to comprise an optional power coil 114 and a communication coil 116. Power coil 114 is coupled by wires (not shown) to driver circuits 34, and generates an electromagnetic field to provide power inductively to power coils 74 in sensor 70 (FIG. 11A), as described above. (When a battery-powered sensor is used, the power coil is not required.) Communication coil 116 receives signals transmitted by communication coil 76 in sensors that are implanted in the patient's body, as well as from tool sensor 104. Communication coil 116 may also be used to transmit control signals, such as a clock signal, to the implanted sensors and tool sensor.

Communication coil 116 is coupled by wires (not shown) to computer 36. The computer processes the signals received from communication coil 116 in order to determine the locations and orientations of the sensors. Coils 114 and 116 may be printed on the surface of pad 110, as shown in FIG. 14A, or they may alternatively comprise printed circuit traces or wire-wound coils contained inside pad 110.

FIG. 14B schematically shows a working volume 118 created by field generator coils 32 when driven by driver circuits 34. The surface of the working volume represents the outer limit of the region in which tracking system 20 is able to determine sensor coordinates to within a certain accuracy. The required accuracy is determined by functional considerations, such as the degree of positioning precision required by surgeon 22 in performing the surgical procedure at hand. Typically, the outer surface of working volume 118 represents the limit in space at which tracking accuracy drops to the range of 1-2 mm. Tilting the field generator coils, as shown in FIGS. 14A and 14B, typically lowers the centroid of the working volume. Because pad 110 is rigid, it cannot be raised and lowered or tilted, as can structure 40 in FIG. 1. Pad 110 may, however, be slid in and out of table 112 in order to shift the position of working volume 118 along the table, so that the working volume intercepts the bone or portion of the bone on which the surgeon in to operate.

FIG. 15 is a schematic, pictorial illustration showing how reference structure 40 may be adjusted for use in surgery on a knee 120 of patient 26, in accordance with an embodiment of the present invention. The patient lies on an operating table 122, which folds as shown in the picture to give the surgeon convenient access to the patient's knee joint. Base 44 of structure 40 tilts accordingly, so that the working volume of field generator coils 32 encompasses the area of knee 120, while still permitting the surgeon unimpeded access to the area.

FIG. 16 is a schematic, pictorial illustration showing a reference structure 130 for supporting field generator coils 32, in accordance with another embodiment of the present invention. Structure 130 comprises arms 132, which hold coils 32. The arms are fixed to an articulated boom 134, which permits the height and angle of the field generator coils to be adjusted relative to the position of the patient on an operating table 136. Boom 134 may be carried by a wheeled cart 138, so that structure 130 can be positioned at either side of table 136 or at the foot or head of the table. Cart 138 may also contain computer 36 and/or driver circuits 34. To reduce clutter over operating table 136, structure 130 may be integrated with the overhead surgical lamp 140, as shown in the figure. In this configuration, lamp 140 illuminates the area of the working volume of coils 32. An additional suspended lamp 142 is shown for completeness. Either of lamps 140, 142 may correspond to lamp 6 of the operating room system.

FIG. 17 is a schematic, pictorial illustration showing a reference structure 150 supporting field generator coils 32, in accordance with yet another embodiment of the present invention. Structure 150 comprises an articulated boom 154, which holds arms 152 to which coils 32 are attached. In this embodiment, structure 150 is tilted and positioned over the area of the patient's knees, to provide functionality similar to that shown in FIG. 15.

FIGS. 18A and 18B are schematic, pictorial illustrations showing another reference structure 160, in accordance with a further embodiment of the present invention. Structure 160 comprises a semicircular holder 162 for field generator coils 32, which is mounted on a base 164. Whereas the reference structures in the embodiments shown above are configured to position coils 32 in a plane that is roughly parallel to the long axis of the bone to be operated upon (such as the femur or the fibula), the plane of structure 160 is roughly perpendicular to this axis. Typically, for proper positioning of the working volume, structure 160 is placed so that the bone axis passes through the circle defined by the positions of coils 32, i.e., so that holder 162 partly surrounds the bone axis.

Structure 160 may be mounted on a cart 166 with wheels, enabling it to be positioned either at the foot (FIG. 18A) or head (FIG. 18B) of table 122. An adjustment slot 167 or other mechanism in base 164 permits holder 162 to rotate about the patient. A hinge permits base 164 to tilt, while telescopic legs 170 permit the entire structure to be raised or lowered. Structure 160 may thus be positioned flexibly, at the convenience of the surgeon, depending on the type of procedure that is to be carried out. The configuration of FIG. 18A, for example, may be convenient for hip surgery, while that of FIG. 18B is convenient for knee surgery.

FIG. 19 is a schematic, pictorial illustration showing a magnetic tracking system 180 for use in surgery, in accordance with still another embodiment of the present invention. In this embodiment, the tracking system is integrated into an operating table 182. A reference structure 184 is fixed to the underside of table 182 by an articulated mount that permits structure 184 to be rotated, tilted, raised and lowered, so as to position field generator coils 32 as required for the surgical procedure in question. A telescopic base 186 of table 182 contains driver circuits 34 and computer 36. Positions and orientations of position sensors, implants, tools in planning and IGS software application GUIs are shown on display 12, which is likewise integrated with table 182. System 180 thus permits the surgeon to operate with only minimal added encumbrance due to the use of magnetic position tracking.

Although the embodiments described hereinabove relate specifically to tracking systems that use time-varying magnetic fields, the principles of the present invention may also be applied, mutatis mutandis, in other sorts of tracking systems, such as ultrasonic tracking systems and tracking systems based on DC magnetic fields.

As illustrated in FIGS. 11A and 11B, the marker 70 is hermetically sealed by encapsulation in a sealant or encapsulant material 71. Preferably the sealant provides any, some or all of the following shielding properties: mechanical shock isolation; electromagnetic isolation; biocompatiblility shielding. The sealant can also help to bond the electronic components of the marker together. Suitable sealants, or encapsulants, include USP Class 6 epoxies, such as that sold under the trade name Parylene. Other suitable sealants include epoxy resins, silicon rubbers and polyurethane glues. The marker can be encapsulated by dipping the marker in the sealant in a liquid state and then leaving the sealant to set or cure.

With reference to FIGS. 20A to 20E there is shown a housing part 200 of a further embodiment of an implantable marker part of the present invention. Housing 200 has a generally right cylindrical body portion 202 with a distal end 204 and a proximal end 206. The housing 200 has a cavity 208 defined therein for receiving an encapsulated marker 70 to provide an implantable marker. This embodiment of the implantable marker is percutaneously implantable. The implantable marker can be implanted in a patient's bone by injection through the skin of the patient, without requiring a preliminary incision.

The distal end 204 has a generally tapered shape and includes a tip 210 for self-locating the implantable marker in a hole in a bone in use as will be described in greater detail below.

The proximal end 206 of the housing has a substantially square shaped formation 212 which provides a connector for releasably engaging with an insertion tool as will be described in greater detail below. The proximal end 206 has a bore 214 passing there through for receiving a thread or suture which can assist in removal of the implantable marker as will also be described in greater detail below. It will be appreciated that the connector formation 212 can have other shapes which allow an instrument to be releasably connected thereto so as to impart rotational drive to the implantable marker.

For example the connector can have any polygonal shape, such as triangular or star shaped, and can also have a curve shape, such as an oval or elliptical shape. In alternate embodiments, the connector can also be in the form of a slot, rib or lip for engaging with a matching connector formation on the end of insertion tool. As illustrated in FIG. 20A, the corners of the connector formation 112 are preferably chamfered in order to facilitate engagement of the connector and insertion tool.

The self-locating tip 210 can be provided as an integral part of housing 200 or can be provided as a separate part which is subsequently attached to housing 200. For example tip 210 can be moulded on to the distal end 214 of housing 200, mechanically fixed thereto or attached using an adhesive or any other suitable techniques, depending on the materials of the tip 210 and distal end 204 of housing 200. Tip 210 can be made of a resorbable material so that the tip is resorbed into the bone of a patient over time. In one embodiment, the resorbable material is polylactic acid although other resorbable materials can be used. In some embodiments, the tip can be made of a biodegradable material.

Housing 200 has an outer surface 216. A screw thread 218 is provided on the outer surface and extends along substantially the entire length of the housing body. Screw thread 218 interacts with surrounding bone in use to anchor the implantable marker in the bone material so as to retain the implantable marker securely in place when implanted.

In one embodiment, the profile of the thread is selected so as to be not too sharp and not too blunt. It has been found that too sharp a thread profile, while providing a good cutting action into the bone, can cause the bone to retreat from the thread thereby reducing the retention of the implant in the bone. A blunter thread profile does not provide as good a cutting action as a sharper profile, but provides improved retention of the implant in the bone, as the surrounding bone has a reduced tendency to resorb from the more rounded thread. As best illustrated in FIGS. 20B and 20C, which show cross sections along the longitudinal axis of the housing 200, the cross sectional shape or profile of the thread has a rounded or flattened apex and can be considered to have a generally rounded trapezoidal cross section. In one embodiment, the radius of curvature where the thread joins the body can be of order 100:m. In one embodiment, the thread profile can vary along the length of the body. The thread can have a sharper profile toward the distal end of the housing so as to provide a good initial cutting action. The thread profile towards the proximal end of the housing can have a more rounded, flatter profile, so as to provide a better anchoring mechanism. The thread profile can vary continuously along the longitudinal axis of the housing or alternatively, can vary discretely and multiple different thread profiles can be provided in order to balance the requirements of a good cutting action and good anchoring and retention of the implantable marker.

The housing 200 can be made of a variety of materials and can be constructed in a variety of ways. In one embodiment, the housing is made of an X-ray opaque material so that the implantable marker will be easily identifiable in X-ray images. It is also preferred if the material of the housing is easily visualisable in CT and/or MRI scan images. The housing can be made of ceramic materials, e.g. zirconium, alumina or quartz. The housing can be made of metals, e.g. titanium and other bio-compatible metals. The housing can be made of alloys, e.g. Ti₆Al₄V. The housing can be made of plastics materials, e.g. epoxy resins, PEEKs, polyurethanes and similar. Also, the housing can be made of combinations of the above materials and the housing can be made of component parts made of different types of materials, selected from the above mentioned materials at least. The component parts can be joined together using any suitable technique, such as brazing, welding or by using suitable glues or adhesives.

In one preferred construction, the housing is assembled from three elements, in which the distal end 204 is in the form of a titanium cap, a portion of the body 202 is in the form of a titanium collar and the proximal end 206 is in the form of a ceramic end cap. The titanium collar is joined to the ceramic proximal end portion by brazing, the encapsulated marker is inserted within the body and finally the distal end cap is assembled over the end of the marker and laser welded to the titanium collar. The marker is positioned with the RF power antenna toward the proximal end and the sensor coils toward the distal end of the housing.

In another embodiment, the housing is made from two ceramic parts which are then laser welded together along a joint extending along the longitudinal axis of the housing. In other embodiments, the housing can be provided by moulding the housing around the encapsulated marker for example by moulding a plastics material around the marker. The internal shape of the mould can be used to define the outer shape of the housing. Alternatively, the outer shape of the housing can be defined by subsequently machining the material moulded around the marker.

Housing 200 wholly encloses the marker and further hermetically seals the encapsulated marker. It is preferred if a small volume, e.g. approximately 1 mm³ of air is provided as free space in the hermetically sealed housing so as to allow for expansion owing to changes in temperature. It is also preferred to include a small amount, e.g. 1 mm³ of hygroscopic material to absorb moisture from the internal atmosphere of the housing. Suitable materials include MgS and silica gel.

The housing can have a length in the range of approximately 10 to 16 mm and a diameter in the range of approximately 3 to 6 mm. In one embodiment the housing 200 (without tip 210) has a length of approximately 14 mm and an outer diameter of approximately 3.6 mm (4.5 mm from the thread tips).

In the embodiment illustrated in FIGS. 20A to 20E, the thread 218 provides a bone anchor. The bone anchor can be provided by other mechanisms. The bone anchor can be provided by other formations on the surface of the housing. The bone anchor can also be provided by the surface of the housing and/or the surface of any formations on the housing, by suitably treating or otherwise configuring the surface of the housing so as to promote bone on growth on to the outer surface and/or formations of the housing. Examples of bone anchor formations, include screw threads, barbs, ridges, ribs and other large scale formations which can be provided on the outer surface of the housing.

In other embodiments, a rough outer surface can provide a bone anchor and a rough outer surface can be realised by using a mould having a roughened inner surface so that the outer surface of the moulded housing is roughened. In other embodiments, the surface finish of the housing can be used to provide a bone anchor e.g. by blasting the surface with titanium to provide approximately 12 micron roughness. The material with which the surface of the housing is blasted can vary and is typically the same material as the material of which the housing is made. For example a ceramics housing can be blasted with ceramics materials to provide enhanced roughness to promote or otherwise facilitate bone on growth.

In another embodiment, the surface of the housing can be treated to promote bone on growth by sintering small balls or particles of material on to the outer surface of the housing. For example, balls of approximately 250 micron diameter metal particles can be sintered to the outer surface of the housing. Such a surface coating is provided under the trade name Porocoat by DePuy International Limited of Leeds, the United Kingdom. In other embodiments, a mesh can be provided on the outer surface of the housing to promote bone on growth. In other embodiments, a hydroxy apatite coating can be provided on the outer surface of the housing. Other forms of coating can also be provided so as to promote or otherwise facilitate bone on growth.

A further embodiment of the marker includes a transducer or other sensor for detecting a property in the region or area around where the marker has been implanted. Transducer or sensor generates an electrical signal representative of the local property of the body and the signal is processed by circuitry 78 for transmission back to the tracking system using antenna 76. In other embodiments, the signal from the transducer can be transmitted back to the tracking system using a wire line system, e.g. a electrical conductor or optical conductor, such as a fibre optic cable.

The transducer or sensor can be of many types, depending on the property to be measured. For example the body transducer 380 can be a pressure transducer, a stress transducer, a temperature sensor, which provides a measure of the local temperature, a biological activity sensor, which provides an indication of a biological activity (e.g. osteoblast activity) or a chemical sensor, which provides an indication of a local chemical property (e.g. pH). Other types of sensors for different kinds of properties can of course be used also.

The marker can be wholly encapsulated by encapsulant material and/or a housing, or apertures may be provided in the encapsulant and/or housing in appropriate places to allow any sensor or detector parts of the transducer to have access to the local region of the body that it is intended to measure.

With reference to FIG. 21 there is shown a schematic cross section of a further embodiment of an implantable marker 230. In this further embodiment, the implantable marker comprises encapsulated marker 70 and housing 232. Encapsulated marker 70 is secured within a cavity 234 defined by a body part 236 of housing 232. A distal end 238 of the housing 232 is provided in the form of a self-cutting, bone penetrating tip which is sufficiently sharp to cut through soft tissue and penetrate into bone. The self-cutting tip 238 can be in the form of a trocher or other sharp shape capable of penetrating bone.

The encapsulated marker is not wholly enclosed in this embodiment and a part of the marker, including the power coil and antenna is exposed. The sensor coil part of the marker is located within the cavity of the housing. This way, when the implantable marker is implanted within a bone, the sensing coils are located within the bone and surrounded by bone so that the position indicated by the sensing coils corresponds to a position within the bone adjacent to the surface of the bone.

Implantable marker 230 has a bone anchor in the form of a plurality of barbs 240 located around the periphery of the housing 232. Each barb is in the form of a rigid member 242 mounted by a pivot 244 to the body of the housing. Pivot 244 includes a spring, or other resilient biasing device, which biases the member 242 away from the stowed state illustrated in FIG. 13 and toward a deployed state as illustrated by dashed lines 246. In the deployed state, the element 242 acts as a barb which resists movement of the housing out of the bone so as to retain the implantable marker within the bone. Bone anchor 240 can be provided in other forms. For example the bone anchor can be provided as a continuous part of housing 232, in the form of a leaf spring which is biased towards the deployed state so as to act as a barb. Alternatively, the bone anchor can be in the form of teeth, serrations or other barbed formations on the outer surface of housing 232 which are permanently in a “deployed” state and which do not have a stowed state.

The implantable marker 230 is particularly suited for use in a “push fit” insertion method as will be briefly described below.

With reference to FIG. 22, there is shown a further embodiment of an implantable marker 250. Implantable marker 250 has a housing similar to that shown in FIGS. 12A to 12E, but the distal end 252 has a tip 254 bearing a self-tapping screw thread 256. Self-tapping screw thread 256 allows this embodiment of the implantable marker to be used in a self-tapping implantation method as will be described briefly below.

With reference to FIG. 23, there is shown a flowchart illustrating an embodiment of a method 260 for percutaneously implanting an implantable marker according to an aspect of the invention. FIGS. 24A to 24D show various instruments and tools suitable for use in the percutaneous implantation method 260.

Instrument assembly 280 includes a guide instrument 282 having a housing 284 and an elongate guide tube 286 having a guide channel extending along a longitudinal axis thereof. There is also provided a drill instrument having an elongate body with a circular cross-section and having a drill bit 288 at a distal end having a skin piercing tip 290 with a trochar form. FIG. 24A shows the distal end of the drill instrument extending from a distal end 292 of guide tube 286 in greater detail. A drive mechanism 294 is attached to a proximal end of the drill body and includes a powered drive, e.g. electrical motor, and a switch or button 296 operable by a user to impart rotational motion, in either direction to the body of the drill.

At step 262, the instrument assembly 280 is pushed through the skin 300 of the patient by a user pushing on the instrument assembly in the direction indicated by arrow 302. The skin piercing tip 290 of the drill bit penetrates the outer surface of the skin and allows the drill and guide tube 286 to be inserted through the patient's skin. The drill can move in the guide channel relative to the guide tube 296 and the guide tube is pushed towards the bone until the distal end 292 of the guide tube engages with the outer surface of the bone 304 of the patient. The distal end of the guide tube 292 bears teeth or other serrated formations which can be pushed into the bone so as to pliably position the guide tube and so as to prevent rotation of the guide tube 286.

Then at step 264, as illustrated in FIG. 24B, a hole is drilled in the bone 304 by the user operating switch 296. Then at step 266, after a hole has been drilled in the bone 304, the drill is withdrawn along the guide tube until the drill bit is located within housing 284 of the guide instrument 282. This configuration of the instrument assembly 280 is illustrated in FIG. 16C. FIG. 24C shows an enlarged view of housing 284 and the body of drill instrument 291 extending there from. Within housing 284, there is provided a cartridge, or magazine, including a plurality of implantable markers 200. The drill instrument is removed from the housing 284 and an adapter, or connector, is attached over the end of the drill bit 288. The adapter has an end with a square recess therein for releasably engaging with connector 212 of the implantable marker housing. With the adapter attached over the drill bit, an insertion tool is provided. In alternate embodiment, a separate insertion tool is provided corresponding generally to the drill described, but rather than having a drill bit at the distal end, a connector is provided which can releasably engage with the connector 212 of the implantable marker housing. In a further alternate embodiment, a plurality of assemblies of implantable markers and prospective adapters are provided in housing 284.

Irrespective of whether a separate insertion tool is provided or whether the adapter and drill provide the insertion tool, at step 268, the end of the insertion tool/adapter is engaged with a one of the implantable markers in housing 284. FIG. 24C shows an enlarged view of the distal end of the insertion tool/adapter with the implantable marker 200 releasably connected thereto. The insertion tool is pushed along the guide channel of the guide instrument 282 as indicated by arrow 302 and the implantable marker is driven into the pre-drilled hole by the user pressing the button 296. In an alternate embodiment, the implantable marker can be manually screwed into the pre-drilled hole, using a tool similar to tool 28 described previously. FIG. 24D illustrates the implantable marker 200 having been percutaneously implanted within a cortical region of bone 304.

At step 272, the instrument assembly is withdrawn from the patient's skin. At 274, the user can then percutaneously implant a further implantable marker if required, in the same manner, as indicated by line 276. For example, a first implantable marker may be implanted in the tibia and a second implantable marker may be implanted in the femur, so as to allow the positions of the tibia and fibula to be tracked during a computer aided surgical procedure. If it is determined at step 274 that no further implantable markers are required in the patient's bones, then the method ceases at step 278.

With reference to FIG. 25, there is shown a method 310 for removing an implanted implantable marker 200 from the bone 304 of a patient through the patient's skin 300. Steps of the method are illustrated in FIGS. 26A to 26D. As illustrated in FIG. 26A, the implantable marker 200 can have a length of suture 330 passing through channel 214 in the proximal end of the implantable marker housing. The length of suture can be used to close the point in the skin where the implantation instruments puncture the skin's surface. Stitches 332 in the skin 300 of the patient therefore approximately indicates the location of the implantable marker 200 in the bone 304.

Method 310 begins at step 312 and initially a user of the method locates the approximate position of the implantable bone marker at step 314. The stitches are undone 332 and the ends of the suture 330 are obtained.

As illustrated in FIG. 26B, a set of tools or instruments similar or the same as those used for implanting the implantable marker can be used to remove the implantable marker. Either an insertion tool or a drill bearing an adapter to provide the insertion tool can be used. FIG. 26C shows the end of the insertion tool or drill bearing an adapter 334. As can be seen in FIG. 26C, the end of the insertion tool/adapted drill 334 includes a square cross-section recess 366 having an aperture 338 in communication with a bore extending to a groove or channel 340 in the outer surface of the insertion tool. The free ends of suture 330 are passed through aperture 338 and out into channel 340 at step 316.

After the suture 330 has been engaged with the end of the insertion tool at step 316, then at step 318, the insertion tool assembly is pushed through the skin of the patient while applying tension to the free ends of the suture 330 so as to guide the instrument assembly toward the connector 214 on the proximal end of the implantable marker 200. At step 320, the distal end of the insertion tool is attached to the implantable marker and switch 296 can be operated so as to unscrew the implantable marker from the bone 304. The sutures 330 are kept under tension so as to keep the implantable marker connected to the distal end of the insertion tool. In an alternate embodiment, the implantable marker can be removed manually using a tool similar to tool 28 inserted through guide tube 286. At step 322, once the implantable marker has been unscrewed from the bone 304, the instrument assembly and implantable marker are withdrawn through the patient's skin 300. The user can then determine whether there are any further implantable markers to be removed at step 324, and if so, the further implantable markers can be removed using the same method, as indicated by line 326. When it has been determined that all the implantable markers have been percutaneously removed, then at step 328, the method of removal 310 ends.

The implantable markers described above are trackable by the tracking system and therefore once they have been percutaneously implanted in the patient's bones, the position of the patient's bones can be tracked and displayed during a computer aided surgical procedure. It will be appreciated that no invasive surgical steps are required in order to implant the markers and therefore the implantable markers can be implanted before a surgical procedure and so can be carried out as a clinical, or out-patient procedure. For example, the implantable markers can be percutaneously implanted in the patient's bones several days or weeks before the surgical procedure. IN other embodiments of the method, the markers are percutaneously implanted with the patient in the operating room but before any incision related to the orthopaedic surgical procedure has taken place.

With reference to FIG. 27 there is shown a flowchart illustrating a computer aided surgical procedure 650 according to the present invention. The method begins at step 652 and at step 654, bone markers are percutaneously implanted in the bones of the patient adjacent the body part on which the surgical procedure is to be carried out. For example, if a hip replacement operation is to be carried out, then a bone marker is implanted in the pelvis and a bone marker is implanted in the femur. If a knee replacement operation is to be carried out, then a bone marker is implanted in the femur and a bone marker is implanted in the tibia. More than one bone marker can be implanted in each bone, if appropriate. Percutaneous implantation of the bone markers can be carried out as an out patient procedure and so can be considered a pre-operative step which can be carried out days or weeks in advance or with the patient in the operating room. In other embodiments, the implantation of bone markers is not percutaneous and is carried out in the operating room via incisions in the patient's body.

At step 656, any pre-operative imaging of the patient can be carried out, such as CT scan, X-ray, ultrasound or X-ray fluoroscopy imaging. The patient image data 624 is stored in storage device 14 so as to be accessible subsequently. It will be appreciated that in some embodiments, pre-operative imaging 656 is not required and therefore in some embodiments, step 656 is optional.

At step 658, the surgeon can carry out pre-operative planning of the surgical procedure using a surgical planning software application. The surgical planning application allows the surgeon to determine the appropriate size of implant to use and the appropriate positions and orientations at which to fix the implant in order to provide appropriate orthopaedic performance of a patient. The results of the planning are saved as a surgical plan for subsequent use during the computer aided surgical procedure. In other embodiments, no pre-operative planning is carried out and instead an intra-operative plan is created and therefore 658, in some embodiments, is optional.

All or some of the above steps can be carried out outside the operating room in some embodiments. At step 662, the patient is registered with the reference frame of the orthopaedic operating room using a suitable registration procedure. A variety of different registration procedures can be used in order to register the position of the patient's body parts in the operating room with images of the patient's body parts. Various methods for registering the patient will be described in greater detail below. After the position of the patient has been registered, then at step 666 the stored surgical plan is merged with the registered patient position so that the surgical plan is now registered in the reference frame of the operating room.

In an alternate embodiment in which the pre-operative planning is not carried out, then at step 664, after the patient has been registered, surgical planning is carried out using the registered patient body position and so a registered surgical plan is provided at step 666.

Some registration methods can require access to the patient's bones and therefore in some embodiments, step 662 corresponds to an intra-operative procedure whereas in other embodiments, registration step 662 can be considered a pre-surgical operation procedure. At step 668, the surgical procedure is either begun or continued and, using the surgical plan, the surgeon carries out the surgical operation using various marked instruments, tools and implants with reference to the various display screens which provide a real time indication of the positions of the instruments, implants and body parts so as to provide an image guided surgical environment for carrying out the method.

While carrying out the computer aided surgical procedure, the surgeon can select to view various images on various of the display units provided throughout the operating room by the orthopaedic operating system 1 so as to access as much useful information in visualisable form as required in order to carry out the procedure. Navigation of the tools, instruments and implants can be carried out using the wireless magnetic tracking system and/or the infrared tracking system.

At step 670, immediately after completion of the implantation part of the surgical procedure, the surgeon can assess the success of the surgical procedure e.g., by comparing an actual image of the surgical site with an indication of the planned position of the implants, or by articulating the joint and comparing the behaviour of the patient's joint with a theoretic, planned or pre-operative joint behaviour. This post-operative assessment can be carried out either before or after the surgical wound has been closed.

In some embodiments, the bone markers can be left in the patient's bones to allow for future assessment of the orthopaedic performance of the patient's body. In other embodiments, at step 672, the implanted bone markers can be removed while the surgical wound is still open or alternatively percutaneously, using the instruments and methods previously described. The bone markers can be removed in the operating room, or alternatively, after the patient has been removed from the operating room in a clinical out patient procedure. The overall method 650 then ends at step 674.

Before describing a particular computer aided surgical procedure which can take advantage of the implantable bone markers described above, a number of trackable instruments and tools will be described. These instruments or tools bear on, or in, them a marker, similar to marker 90. Alternatively, they may include an inductively RF powered marker such as marker 70. The markers can be encapsulated in a specific encapsulant material or can be encapsulated, e.g. by being moulded into, a part of the instrument or tool. Alternatively, the marker is attached to the tool and located within a cavity of the tool, in a manner similar to that of tool 28 as illustrated in FIG. 13 above.

With reference to FIG. 28, there is shown a marked pointer tool, sometimes also referred to as a probe, 360. The pointer 360 has a handle 362 which incorporates the marker which is trackable by the tracking system. Handle 362 can be made of a plastics material such as PEEK. Handle 362 has a elongate, substantially straight pointer element 364 extending there from and having a curved tip part 366 at a distal end of the pointer 360. The pointer element 364 is can be made of a metal or alloy material such as 3/16. The curved tip 366 of pointer 360 makes the pointer ergonomically more useable by a surgeon so as to identify anatomical features of the body or parts of implants, or other instruments or tools.

Pointer 360 can be used so as to digitise the surface of a body part, e.g. a part of a bone as part of registering that bone with the coordinate frame of the tracking system. In one embodiment, the marker is positioned in the handle 362 with a set of sensor coils concentric with the longitudinal axis of the pointer element 364. In this way, the orientation of that set of sensor coils substantially corresponds to the orientation of the longitudinal axis of the pointer. The positional relationship between the free end of tip 366 and the position of the marker in the pointer 360 is stored in the tracking system. Therefore when the tracking system identifies the marker, using the transmitted marker ID information, the tracking system can automatically determine the position of the tip of the pointer 366 in the reference frame of the tracking system.

With reference to FIG. 29 there is shown a plane instrument 370 bearing a marker trackable by the tracking system. The marked trackable plane 370 includes a handle part 372 and a plane or cutting part 374. In one embodiment, handle part 372 is made from a plastics material, such as PEEK, or carbon fibre reinforced PEEK. A trackable marker is disposed within handle part 372. A motor is also provided in the handle part, having a switch operable by a user, so as to drive a cutting part of the plane so that the plane can be used to resect a bone and leave a flat resected bone surface.

With reference to FIG. 30, there is shown a burr removal tool 380. Tool 380 includes a marker so that the tool is trackable by the tracking system and so can be used in a navigated or image guided surgical procedure. The tool 380 includes a handle 382 similar to a pistol grip having a switch 384 operable by a user. A body part 386 of the tool has a kinked tubular member 388 extending there from with a tip 390 at a free end thereof. A rotatable or otherwise moveable cutting surface 392 is exposed at tip 390 and is driven by a drive mechanism within the tool 380. Tip 390 also includes a closure mechanism such as an iris or eyelid type shutter.

In use, tool 380 can be used to form a bone surface to a preferred shape or profile or to otherwise remove unwanted bone material. By operating switch 384, the cutting surface 392 is driven and can be played across the bone surface so as to cut the bone surface to the desired shape or profile. The tracking system identifies the marker within the tool using the transmitted marker ID data and the tracking system is pre-programmed with the positional and orientational relationship between the marker and the cutting surface 392. Using planning software, a preferred shape or form of a bone surface can be identified pre or intra-operatively. Then in order to generate that bone surface, the tool can be moved over the bone and the tracking system can detect the position of the tool and allow the tool to cut away the bone surface until the tracking system determines that the position of the cutting element 392 corresponds to the desired position of the bone surface at which time the shutter can be actuated so that the tool 380 no longer cuts the bone surface.

Hence, in this way, the tool can be used to allow the surgeon to easily cut the bone to a preferred shape or profile merely by running the tip of the tool 390 over the bone with the tracking system and computer aided surgical system starting or stopping the cutting action of the tool as appropriate. In another embodiment, no shutter or closure mechanism is provided and instead, driving power is no longer supplied to the cutting element 392 so as to provide the same effect.

With reference to FIG. 31 there is shown a perspective view of a tensioning device or tensor 400. The device 400 includes a top plate 402 and a bottom plate 404 made from a biocompatible metal, or high tensile polymer composite, such as a Ti alloy or stainless steel (for example Ti6AL4V or 300 series stainless steel). The top plate 402 has a femur engaging surface 403 and the bottom plate has a tibia engaging surface 405. A link arm 406 links the top and bottom plates and is connected to each plate by a pivot. The link arm is pivotally connected to the bottom plate 404 by a first pivot 407 including a pivot pin 408 (made from silver steel) passing through engaging pivot formation parts of a first end of the link 406 and the bottom plate. The link arm is also pivotally connected to the top plate 402 by a second pivot also including a pivot pin passing through engaging pivot formation parts of a second end of the link 406 and the top plate. Link arm 406 can be made of the same or similar materials to those of which the plates can be made.

The arm 406 links the top and bottom plates in such a way as to allow the top and bottom plates to separate relative to each other to a predetermined maximum distance. A single spring is fitted between the plates and engages interior surfaces of the plates. The spring provides a biasing mechanism to controllably force the tensor plates toward an open or expanded configuration in which the device is extended along the longitudinal axis of the knee joint when in flexion. A spring force in the range of from substantially 6 kg to 12 kg can be used.

The device is used is to distract the femur from the tibia to establish the correct mechanical loading across the knee joint. The device can be used in an image guided surgery uni-condylar knee replacement as will be described below. The device is introduced into the knee joint after the tibia has been cut and before the femur is cut using an introducer tool which closes or compresses the tensor, and which is then slowly released to contact both the tibia and the femur. The tensor device 400 is placed on a resected part of the tibia and is oriented with its longer dimension in an anterior-posterior direction and its shorter dimension in a lateral-medial direction and with the straight edge of the plates toward the middle of the knee. The bottom plate is placed in the same position as the tibial component will be positioned. The device provides a known force to gap relationship. The tensor device opens and closes with the force of the ligaments of the knee during flexion and extension. When in place, the tibia is flexed and extended and the femur to tibia distances are recorded using the image guided surgery software. From this information the surgeon can decide on and plan the femur cut height to restore the correct joint gap. Hence the device allows the knee joint to be restored having a more correct tension and femur to tibia rotation.

With reference to FIG. 32, there is shown a compression tool 430 for holding tensor device 400 in a compressed state. Compression tool 430 generally has the construction of a pair of forceps, or pliers, having a first arm 432 connected by a pivot 434 to a second arm 436. Compression tool 430 has an upper nose 438 and a lower nose 440. Lower nose 440 has a ridged formation 442 on an inner surface thereof for engaging in a recess or channel 444 in an under side of the bottom plate 404 of the tensor device 400.

The first handle part 432 and second handle part 436 are made from a suitable surgical material, such as aluminium 7075. The pivot 434 is also made of a suitable surgical material, such as silver steel. The upper nose 438 and lower nose 440 are also made from a suitable surgical material, such as an alloy, such as Ti6Al4V.

In use, the handles 432, 436 of compression tool 430 are displaced apart opening the mouth of the tool which is engaged about the tensor device 400 with ridge 442 engaging in channel 444. The handles 432, 436 are then closed by the surgeon and the mechanical advantage provided by the leveraged effect of the handles allows a significant compressive force to be applied to tensor device 400 so as to compress the tensor device 400 into a compressed configuration. The tensor device can then be inserted between the femur and resected tibial surface and positioned therein. The handles 432, 436 are then opened and the compression tool is slid away from the tensor device 400 at a direction generally along the axis of channel 444 leaving the tensor device 400 in situ between the femur and resected tibia.

With reference to FIGS. 33A to 33C there is shown a marked orthopaedic implant 450 providing a prosthetic part of a knee joint. Implant 450 is used to replace a single condyle of the femur and the corresponding bearing surface of the tibia. FIG. 33A shows a perspective view from the anterior of the uni-condylar implant 450, FIG. 33B shows an anterior elevation of the implant 450 and FIG. 33C shows a cross-section along line A-A of FIG. 33B.

The prosthetic implant 450 includes a femoral component 452 and a tibial component 454. Tibial component 454 includes a tibial tray part 456 and a bearing part 458 fixedly attached to the tibial tray 456 by retaining formations.

Femoral component 452 has a continuous smooth outer bearing surface 460. A keel 462 extends along the middle of the femoral component between a toe end 464 and a heel end 466. A hollow locating pin or peg 468 extends away from the heel 462 at a generally centrally location. Peg 468 has a cavity within it which receives a marker 70 so that the position and orientation of the femoral component can be tracked by the tracking system.

An inner bone contacting side of the femoral component has four segments 472, 474, 476, 478 each presenting a substantially flat surface to a suitably prepared femur. Peg 468 is received in a hole or cavity in the prepared femoral head and keel 462 is received in a anterior-posterior groove in the femur. Peg 468 helps to locate the femoral component and groove 462 helps to resist twisting of the femoral component relative to the femur.

As illustrated, uni-condylar implant 450 is for a lateral condyle of a right leg or medial condyle of a left leg and a mirror image implant is also provided for use in replacing the medial and lateral condyles of left and right legs respectively. As illustrated, the marker 470 is aligned with a one of its sensor coils aligned with the longitudinal axis of the femur. The marker can be encapsulated in an encapsulant material and/or partially or wholly enclosed in an outer housing before being secured within the cavity of peg 468. Preferably the marker is an RF induction powered marker to ensure that power can be supplied to the marker throughout the lifetime of the prosthetic implant.

Tibial tray 456 has a lower tibia engaging surface 480 with a keel member 482 extending downwardly there from and along the anterior posterior direction. Keel 482 has a cavity in which a further marker 484 is located. Marker 484 is similar to marker 470. At least a one of the sensor coils of marker 484 is aligned with the anterior/posterior axis of the tibial component 454.

Bearing 458 has an upper curved bearing surface 486 which substantially reproduces the shape of the top of the tibia of a normal knee joint. Bearing surface 486 has a generally slightly concave shape. In use, the outer surface 460 of femoral component 452 bears against bearing surface 486 as the knee joint is articulated.

The femoral component 452 and the tibial tray 456 can be made of any suitable bio-compatible materials. Typically, they are made of bio-compatible metals, including titanium and titanium based alloys, steels and cobalt-chromium based alloys. The tibial tray 458 can be made of plastics materials, such as polymeric materials and in particular ultra-high molecular weight polyethylene (UHMWPE).

As best illustrated in FIG. 33C, the femoral component extends around the anterior of the femur to a small extent with only a small toe part 464. The implant allows a large amount of the femoral bone to be preserved as only parts of a single condyle are removed and only relatively small amounts of bone are removed from that single condyle in order to fit the femoral component. Hence a large amount of the original bone material is removed while still providing good orthopaedic performance. In FIGS. 33A to 33C, the marked prosthetic knee implant 450 is shown in a configuration corresponding to the knee in extension.

With reference to FIG. 34 there is shown a flowchart illustrating an embodiment of a computer aided orthopaedic surgical procedure for implanting implant 450, generally designated 680. Various parts of method 680 correspond to various steps of method 650 illustrated in FIG. 27. FIGS. 35A to J are pictorial representations of various parts of method 680. Initially, corresponding to step 654 of method 650, and as illustrated in FIGS. 35A and 35B, a first implantable marker 708 is percutaneously implanted in the femur 710 of the patient. A second implantable marker 712 is percutaneously implanted in the tibia 714 of the patient. It is preferred to implant the implantable bone markers within a few centimetres, e.g. 5 cm, of the surgical site or body part to be treated, in this example, the knee joint.

At step 684, the surgeon uses the surgeon interface 10 to load patient data and any pre-operative data and/or patient scan data and/or images from the data storage device 14. At step 686, the surgeon can select various data items and patient images to be displayed on the wall display unit 8 and/or on the control system screen 12.

At step 688, an auto-registration procedure is carried out by the surgeon selecting this option and entering a command via surgeon interface 10. The auto-registration procedure will be described with reference to FIGS. 36A, 36B and FIG. 35C in particular.

FIG. 36A shows a flowchart illustrating a method 720 for automatically registering an image of the patient's bones with the actual position of the bones of the patient. Method 720 corresponds generally to step 688. The X-ray imaging system 5 is controlled to capture a first image of the patient's knee from a first direction and a second image of the patient's knee from a second direction. Either an X-ray or an X-ray fluoroscopy images can be captured. Then at step 726, a three dimensional model of the patient's bone is created from the two captured X-ray images.

FIG. 36B shows a method 740 for creating a three dimensional bone model corresponding generally to step 726 of method 720. At step 742, the internal shape and size of the patient's bone is determined. In one embodiment, this is done by processing the X-ray images of the patient's bones to determine a major and minor axis of an ellipse corresponding to the internal cross-sectional shape of the patient's bone. The major and minor axes of a plurality of ellipses positioned along the longitudinal axis of the patient's bone can be determined. Using this measure of the internal shape of the patient's bone, a database query is carried out at step 744 to select a generic model of the patient's bone most closely matching the measured shape.

Previously, a plurality of CT scans of a plurality of different bones is carried out and a plurality of generic models of bones of different sizes are created and stored in the database. In this embodiment, a plurality of generic femurs and tibias is created from CT scans of real femurs and tibias and saved in the database. Using the measure or metric indicative of the size of the patient's actual bone, a generic bone model most closely matching the patient's bone is selected from the database at step 744. Then at step 746, the selected generic bone model is morphed, i.e. its size and/or shape is scaled so as to more accurately correspond to the patient's actual bone shape and size. The customised three dimensional model is then used in the rest of the procedure to provide a more accurate model of the patient's bone.

Various methods for creating a 3D model of a patient's bone from 2D images can also be used. For example, methods are described in U.S. Pat. No. 5,951,475 and international patent application publication number WO 01/22368, which are incorporated herein by reference in their entirety for all purposes.

After method 740 has completed, process flow returns to step 728 at which the position of the X-ray system in the reference frame of the operating room is determined. This can be achieved in a number of ways. For example there can be a fixed positional relationship between the X-ray system and the operating room, in which case a calibration of the X-ray system can be carried out which results in a determination of the position of the imaging plane of the X-ray system in the reference frame of the operating room. Alternatively, a marker trackable by the tracking system 3 can be attached to each of the X-ray detectors. There is a known positional relationship between the imaging plane of the X-ray detectors and the markers.

The tracking system can therefore determine the position and orientation of the imaging plane in the reference frame of the tracking system. Therefore the position of the image of the patient's bone in the reference frame of the tracking system can be determined. Hence the position of the 3D image relative to the reference frame of the tracking system can be determined from the positions of the 2D images in the reference frame of the tracking system. FIG. 35C shows a pictorial representation of the 3d model of the patient's knee, derived from the 2D X-ray images, in the reference frame of the tracking system 750.

At step 730, the position of the patient's bones in the reference frame of the tracking system is determined. This is simply a matter of determining the current position of the bone markers 708, 712 in the patient's bones. FIG. 35C pictorially illustrates the positions of the bone markers in the reference frame of the tracking system 752.

At step 732, the 3d representation of the patient's bone is then mapped, in the reference frame of the tracking system, on to the actual detected position of the patient's bone as graphically illustrated by 754 in FIG. 35C. This can be achieved as there is a known position of the imaging plane of the X-ray detectors in the reference frame of the tracking system. Hence the result of method 720 is registration of the 3D model of the patient's bone with the actual position of the patient's bone in the reference frame of the tracking system.

In an alternate embodiment, the implantable bone markers are provided in an X-ray opaque form so that an image of the bone marker or markers is present in the captured X-ray images. Hence the position of the image in the reference frame of the tracking system is known and so an appropriate mapping can be determined and carried out so as to map the 3d bone model derived from the X-ray images on to the position of the patient's bones.

After the auto-registration procedure 720 has completed at step 734, the method returns to step 690 at which a registered surgical plan is generated. In an embodiment in which a pre-operative plan was created, then the pre-operated surgical plan is merged with the registered model of the body part so as to provide a registered surgical plan. In another embodiment, an intra-operative surgical plan is created on the already registered model of the body part.

FIG. 36C shows a flow chart illustrating a method 920 of using the knee replacement planning software and corresponding generally to step 690 of method 680. The planning software application is used to allow the femur and tibia implants to be correctly positioned with respect to each other to minimise implant stress and maximise contact area. A 3d visualisation of the moved joint (kinematic) is provided with superimposed design limits for relative positioning.

A pre-operative assessment of the patient's joint is conducted by extending and flexing the joint and recording the relative locations of the bones using the implanted markers and the tracking system. Having recorded the bone positions, the surgeon then uses the planning application to choose the implants that best the fits the patient's bones. This typically requires balancing anterior/posterior sizing and medial/lateral sizing. The best implant location is then a compromise of size versus best functional position according to the implant design characteristics. The surgeon can then view a virtual model of the flexion and extension positions (kinematic) of the bones versus external/internal rotation of the tibia to femur and select the best compromise for the patient.

As illustrated in FIG. 36C, the femur and tibia are already registered with the system and at step 922 the size of the femur and the size of the tibia are determined the planning program. Then at step 924, the surgeon articulates the knee joint and the positions of the bones are tracked and captured so that the range of motion of the patient's knee joint is captured. The original range of motion is then stored at step 926.

At step 928, the sizes of the tibial and femoral implants are selected and their positions are planned.

One embodiment of the planning process can include the following. Initially, the position of the centre of the femoral head is defined together with the position of the midpoint of the maleolar axis, which between them define the leg mechanical axis. Then the following positions are defined: (i) the epicondylar axis on the femur, (ii) the local distal anatomical femur axis direction, (iii) the distal point of the femur mechanical axis, (iv) the highest and lowest distal points on the femur, (v) the posterior condyle point, (vi) the anterior femur cortex, (vii) the true anterior-posterior direction, (viii) the lowest condylar position on the tibia, (ix) the true anterior posterior direction, and (x) the anterior cruciate ligament point. The mid point of the maleolar axis at the ankle and (ix) define the tibial mechanical axis.

The position of the tibial component can be determined based on: height in relation to the lowest condyle point; anterior/posterior position in relation to (x); anterior/posterior rotation in relation to (ix); medial-lateral position in relation to (ix); and posterior and medial/lateral tilt in relation to the tibia mechanical axis.

The position of the femoral component can be determined based on: height in relation to the highest distal condyle point; anterior/posterior position in relation to the anterior cortex; anterior/posterior rotation in relation to the epicondylar axis, (vi) and in relation to the location of the tibia plan cut; medial-lateral position in relation to (iii); medial-lateral tilt in relation to tibia cut plan and (ii); and posterior tilt in relation to (ii).

FIG. 35E shows a screen shot 750 from a knee replacement surgical planning application as displayed on display device 12 of the tracking system control computer. As can be seen, the 3D model of the patient's bone 752 is displayed to the user together with 3d images of the orthopaedic implants, e.g. image 754 of tibial component 454. The surgeon can vary the position of the implant components relative to the model of the patient's bone and a part of the graphical user interface provided by screen display 750 displays quantitative measures of the position and orientation of the implant 756. Using the planning application, the surgeon can vary the size of the orthopaedic implants and the position of the orthopaedic implants relative to the patient's bone in a number of ways. For example, FIG. 35F illustrates varying the longitudinal axis of the femoral component and FIG. 35G illustrates varying the anterior-posterior axis of the femoral component 452. As well as displaying a graphical representation of the patient's bone, a graphical representation of the current planned position of the orthopaedic implant 758 can be displayed together with graphical representations of a theoretical or preferred position of the implant based on modelling the intended orthopaedic performance of the patient's bones.

When the knee implant sizes have been selected and their positions determined, then at step 930, a virtual range of motion analysis is carried out for the models of the patients bones and using the planned implant sizes and positions. Then at step 932, the virtual range of motion of the patient is compared with the actual range of motion captured previously and at step 934, the surgeon can determine whether the implant sizes and/or positions are appropriate. If not, and further planning is required the processing returns to step 928 as indicated by line 936 and the size and/or positions of the implants can be changed. Steps 928, 930, 932, 934 and 936 can be repeated as often as necessary until the surgeon is satisfied with the surgical plan. Then at step 938, the surgical plan can be saved if surgery is to be carried out later on, or alternatively surgery can be commenced.

After the orthopaedic plan has been completed in step 690, then at step 692, the surgeon carries out an initial incision. In one embodiment, the initial incision is carried out in a navigated manner. The surgical site display device 7 is positioned over the patient's knee and displays an image of the patient's knee to the surgeon. The surgical planning software can then overlay a graphical indication of the position and form of the incision required in order to execute the planned orthopaedic procedure. After having viewed the planned incision position and shape overlayed over the patient's knee, the surgeon can then remove the surgical site display device and make the incision. Using only a single incision helps to make the procedure a minimally invasive one. In one embodiment, the scalpel or incision device bears a trackable marker and the position of the scalpel is displayed on the control screen 12 together with the position of the incision and an image of the patient's knee and these images are used to guide the surgeon to make the appropriate incision.

After having made the navigated incision, at step 694, the surgical site display can be repositioned over the opened surgical site and/or the surgical camera system 6 can be used to capture real time images of the surgical site which the surgeon can select to display on wall display unit 8 and/or on the control unit display 12. The surgeon can also select to display previously captured images of the patient's knee, e.g. CT scan, X-ray, ultrasound or X-ray fluoroscopy images. The surgeon can also display surgical planning information, such as the preferred or planned location of the implants and can overlay and combine these and other images mentioned previously as appropriate for the surgeon's purposes.

Then at step 696, the surgeon begins the implantation procedure during which the positions of instruments, implants and other elements used by the surgeon are tracked by the tracking system and graphical representations of the implants, instruments and other elements are displayed so as to provide a visual guide to the surgeon. The surgeon can select what images and/or combinations of images to display on whichever of the display devices he finds most convenient as indicated by step 698. At step 700, if the surgical procedure has not been completed, then as schematically indicated by line 702, the tracking system continues to track the positions of the instruments, implants and bones at step 696 and the displays are continuously updated to provide a real time display of the elements within the tracking system.

FIG. 37 shows a further embodiment of the method for carrying out a computer aided knee replacement surgical procedure 770, however using a different registration procedure. A number of the steps are the same as those in FIG. 34 and only the different steps will be described. In this embodiment, a bone morphology registration procedure is used rather than a bone image based registration procedure. At step 772, after the surgical site has been opened, the surgeon uses a tracked pointer to capture a plurality of points on the surface of the patient's bone. The surgeon can capture some specific anatomical landmark points and a plurality of points in order to form a network extending over a part of the bone having a characteristic shape. This process is sometimes referred to as digitisation.

Then at step 774, a generic 3D model appropriate for the size of the patient's bone is selected based on the captured points. The model is then aligned with the patient's bone using the captured points which define a characteristic anatomical feature by which the model and bone can be aligned so as to provide a registered 3D model representing of the patient's bone. As the points on the patient's bone have been captured by the tracking system, the position of the patient's bone in the reference frame of the tracking system are known and the image of the patient's bone is automatically registered in the reference frame of the tracking system. Then at step 776, the implant planning application is used to plan the surgical procedure using the registered model of the patient's bone so as to provide the registered surgical plan. The remaining steps are similar to those described previously with reference to FIG. 34.

FIG. 38 shows a flowchart illustrating the navigated and image guided surgical steps carried out by the surgeon in order to implant the prosthetic knee. FIG. 35H shows a screen shot 780 of the navigated surgical procedure application illustrating the display of the patient's bone together with an indication of the position at which a cut should be made in order to implant the prosthetic implant at the planned position. The surgical procedure application is used together with the tracked instruments to allow the instrument positions to be navigated so that the surgeon can accurately position the instruments using the displayed images of the body parts, instruments and planned positions together with video images of the surgical site.

With reference to FIG. 38, there is shown a flowchart illustrating a surgical method for fitting implant 450 to the knee of a patient. FIGS. 39A to 39D show the femur 512 and tibia 514 of the patient and various tools, guides and the implants being used at various stages of method 490. Method 490 is a computer aided surgical method. Prior to the surgical method 490, the patient has a marker percutaneously implanted in the femur and a further marker percutaneously implanted in the tibia. Using the planning software, the surgeon determines the appropriate positions at which to locate the femoral and tibial components of the implant. Navigation and image guided software applications are then used during the surgical procedure in which the positions of the patient's bones, the prosthetic implants and various tools and instruments are tracked by the tracking system and visually displayed to the surgeon.

The surgical procedure begins at step 492 and at step 494 the navigated incision is made in the skin surrounding the patient's knee so as to expose the surgical site. At step 496, the patient's knee joint is opened and the knee is subluxed or otherwise distracted so as to allow access to the top of the tibia. At step 498, a cutting guide 516, bearing a marker, is navigated into position and attached to the tibia at a position to allow a part of the tibia 514 to be resected in accordance with the position determined by the planning software. A cutting tool 518 is then used with guide 516 so as to make the tibial cut and resect a part of the surface of the tibia as illustrated in FIG. 39A. The tensor device is inserted in the knee between the resected tibial surface and the femur using the compression tool as described previously.

At step 500, as illustrated in FIG. 39B, a further marked guide 520 is navigated into the correct position as determined by the planning software and an initial femoral cut of an inferior part of the femur is carried out at step 500 using cutting tool 518. As illustrated in FIG. 39B, the knee joint is in extension.

The femur is then positioned with the knee joint in flexion and at step 502 marked 522 guide is navigated on to the resected part of the femur and attached to the resected part of the condyle by pins 524. Cutting tool 518 is then used to make three femoral angle cuts to remove a posterior part of the condyle 526, a bone part 528 between the resected surface and a posterior surface and an anterior part 530 as illustrated in FIG. 39C using three guide channel parts of guide 522.

After the femoral angle cuts have been made at step 502, at step 504, the tibial and femoral implants are fitted. Using navigated guides and/or marked drills, reamers, broaches and other surgical tools, a channel in the anterior-posterior direction is created in the resected parts of the femur to receive keel 462. The hole is drilled in the resected part of the femur to accept location pin 468. A cavity is created in the resected surface of tibia 514 to accept tibial keel part 482. The tibial and femoral orthopaedic parts are then fitted to the prepared femur and tibia respectively and secured in place, e.g. using bone cement.

Various conventional surgical steps can then be carried out in order to complete the knee reconstruction and to close the incision and then the method is completed at step 506. After the surgical procedure completes at step 506, at step 704 of methods 680 or 770, the surgeon can evaluate the success of the procedure for example by comparing the actual positions of the implants with the planned implant positions and/or articulating the joint and comparing the actual movement of the patient's limbs with a planned or theoretical movement or pre-operative range of motion of the patient's limbs. This can be carried out with the surgical wound still open or with the surgical wound closed. After the surgical wound has been closed, then at step 706 the computer aided surgical procedure ends and then the bone markers can be removed as illustrated in FIG. 35J and corresponding to general method step 672 of method 650.

With reference to FIGS. 40A to 40C, there is shown a prosthetic hip 540 bearing markers to allow the prosthetic hip implanted as part of a computer aided surgical procedure 580 illustrated by the flowchart shown in FIG. 28. The marked prosthetic orthopaedic implant 540 includes a femoral component 542 and a pelvic component 544. FIG. 40A shows a perspective view of the prosthetic hip joint, FIG. 40B shows an elevation of the prosthetic hip joint 540 in a lateral to medial direction and FIG. 40C shows a cross-section along line AA of FIG. 40B.

The pelvic component 544 has a generally concave or cup shape. The pelvic component 544 has a body part 546 with an outer shell part 458 generally in the shape of a part of a sphere and treated to encourage bone ongrowth. A substantially circular aperture 550 is provided in an outer part at the apex of cup 544 for receiving a marker including at least a sensor coil, RF induction power coil, antenna and associated circuitry so that the marker can receive power and transmit its identifier, and position and orientation data to the tracking system. The marker is described in greater detail with reference to FIGS. 41A-41D below. The inner surface of acetabular cup 544 is highly polished and provides an articulate surface having a shape corresponding to a part of a sphere.

The femoral component 542 includes a body part 552 generally in the form of a shoulder having a stem or tail part 554 toward an inferior part of the body and a neck part 556 toward a superior part of the body. A marker 558, similar to marker 470, is provided in a cavity toward a superior part of the shoulder of body 552. Neck 556 tapers slightly toward a free end. A head part 560 is attached to neck 556 by a collar or sleeve member 562. Sleeve 562 has a generally annular shape and provides an adapter by which head 560 is secured to body 552 in a tight push fit manner.

Head 560 has a highly polished surface 562 generally corresponding to a part of the surface of a sphere. An annular channel 564 extends around a longitudinal axis of head 560 and an inner wall 566 defines a cavity 568 within which sleeve 562 and neck 556 are received. Body 552 has an outer surface or shell part 570 extending there around which is configured to encourage bone on growth.

A cavity 572 having a substantially v-shape is provided in an upper part of the shoulder of body 552. Cavity 572 provides a connector by which an impactor tool can be engaged or otherwise attached to femoral component 542 to aid in fitting the implant.

With reference to FIG. 41A there is shown a magnified cross section through the apex of acetabular cup 544 showing an acetabular marker 571 received within cavity 550. FIG. 41B shows a perspective view of the acetabular marker 571, FIG. 41C a transverse cross sectional view of acetabular marker 571 and FIG. 41D a cross sectional view along line AA of FIG. 41D. Acetabular marker 571 has a housing 572 having a convex upper surface and a concave lower surface. The marker surfaces are configured to smoothly continue the surfaces of the surrounding parts of the acetabular cup 544. Housing 572 has a screw threaded portion 573 extending around its periphery which engages with a thread within an inner wall of acetabular cup 544 defining cavity 550. This provides an attachment mechanism by which the marker can be secured to the acetabular cup. In other embodiments, the marker can be attached by an adhesive, brazing welding or by using a mechanical connection such as a push-fit or snap-fit formations.

The housing 571 can be made from an assembly of a ceramic material and a metal or alloy material. Suitable ceramic materials included YTZP (Yttria partially toughened zirconia), Alumina or Zirconia toughened Alumina. Suitable alloys include titanium alloys, such as Ti6Al4V. The join between the ceramic and metal/alloy components can be provided by a combination of a high temperature braze (before assembly of the electronic components) and a laser or electron beam weld (with the electronics in situ). The ceramic parts allow for RF transmission therethrough.

A marker is 577 is provided in the housing. The housing 571 includes three cavities 574, 575, 576 in which the location coil 72, circuitry 78 and power coil 74 of the marker are located. The transmission antenna and connections between the electronics components are also provided in the housing. The electronic modules 72, 74, 78 are substantially the same as those described above for the implantable marker and provide the same functions but configured in a different geometry. Each or all of the marker electronic modules can be pre-encapsulated in an encapsulant material 578, such as an epoxy.

The complete acetabular marker 571 is inserted into the acetabular cup. This can be carried out pre-operatively, during assembly of the acetabular cup, or intra-operatively just prior to, or after, implanting the acetabular cup.

With reference to FIG. 42 there is shown a flowchart of a method 780 for planning the implementation of the hip prosthesis 540 shown in FIGS. 40A to C. This method corresponds to various of the steps of the general method illustrated in FIG. 27. The planning method begins at step 782 and, if images of the patient's body part are not already available, then CT, X-ray, X-ray fluoroscopy or ultrasound images of the body part can be captured at step 784. Then at step 786, 3D models of the patient's body parts, in this instance the pelvis and femur are derived from the images of the pelvis and femur using a process similar to that described previously. That is a generic 3D model of the body part is morphed so as to more closely resemble the actual shape of the patient's body part as determined from the captured images.

Based on the models of the patient's pelvis and femur, the surgeon determines the appropriate implant system to use. As will be indicated below, in some embodiments, other prosthetic hip implant parts, different to prosthetic hip 540, can be used. At step 788, the surgeon selects an initial size of cup implant and stem implant in order to start the planning procedure. At step 790, the surgeon can plan the position of a virtual model of the acetabular cup implant relative to the model of the patient's pelvis. An image of the model of the patient's pelvis and an image of the acetabular cup are displayed to the surgeon together with information indicating the orientation of the cup relative to the pelvis and other useful surgical planning information similar to that illustrated in FIGS. 35E to G in connection with the knee implant. The position of the cup can be based on the inclination and anteversion angles with reference to the sagittal, frontal and transverse planes of the pelvis. The locations of the sagittal, frontal and transverse planes of the pelvis are obtained from the 3D model of the patient's pelvis and an indication of the inclination and anteversion angles, as the orientation and position of the cup is varied, are displayed to the surgeon.

At step 792, the surgeon can consider whether the initially selected cup is appropriate and if not at step 794, the surgeon can select a different cup and plan the position of the differently sized cup at step 790. Steps 790, 792 and 794 can be repeated a number of times in an interactive process until the surgeon has settled on an appropriate cup size that best fits the patient's anatomy.

Planning the position of the cup can involve defining a rotation centre of the acetabulum and an outer diameter of the cup. This can be achieved by identifying multiple points inside the acetabulum of the model of the patient's pelvis and calculating the centre of rotation and outer diameter of the cup based on the acquired points. In an alternate embodiment, the surgeon can digitise the positions of the points on the acetabular cup of the actual patient's pelvis using a tracked pointer. FIG. 42B shows a pictorial representation of the model of the patient's pelvis 791 illustrating the collection of a plurality of points on the surface of the acetabulum and the centre of rotation 793 defined therefrom. FIG. 42C shows a pictorial representation of the pelvis 791, the anatomical centre of rotation of the acetabulum 793, an image of the acetabular implant 795 and the centre of rotation of the acetabular implant 797. Typically, the inclination angle of the acetabular implant would be in the range of approximately 35E to 50E and the anteversion angle in the range of approximately 15E to 30E with respect to the pelvic frontal, sagittal and transverse planes.

At step 796, the position of the stem component 542 is planned. The planning of the position of the stem component 542 is illustrated in FIG. 42D. FIG. 42D shows an image of the model of the patient's femur with an image of the stem implant 542 overlaid thereon. The position of the stem 542 is planned with respect to the axis of the femoral neck and the stem axis obtained from the femoral image data. The stem is located at a position to fit within the medial and lateral flares of the femur and so as to obtain the required varus/valgus, antetorion, anterior/posterior position with respect to the patient's anatomy. In particular, the axis of the femoral shaft 799 is defined in the image of the patient's femur 801 and the long axis of the stem, the stem neck axis and the centre of the head to be fitted to the stem are all defined. The intended resection level 803 is planned and the stem is positioned such that the stem antetorsion follows the natural femoral antetorsion.

The position of the stem is calculated with its long axis co-axial with the longitudinal axis of the femur. A display of any angular difference between these axes can be provided. The stem is also positioned with the medial and lateral flares pressing against the femoral cortex and with the depth of the stem as required such that the leg length will be the same for both of the legs of the patient. The calculated stem antetorsion can be displayed. Step 798 includes planning the position of the stem relative to its depth in the femur in order to provide the required leg length.

Then at step 800, the leg length provided by the planned stem and acetabular cup position is calculated and compared with the pre-operative leg length and the leg length for the other leg of the patient at step 800. Also, the hip offset is calculated and again compared with the pre-operative hip offset of the patient and the hip offset for the patient's other hip. The calculation of the patient's leg length and calculation of the hip offset are illustrated schematically in FIG. 42E. At step 802, the stem size and/or offset provided by the stem can be changed and as illustrated by line 804, any of steps 796 to 800 can be repeated in an interactive process until the surgeon is satisfied with the planned sizes and positions.

At step 806, the range of motion provided by the planned implants can be checked by moving the virtual representation of the patient's femur with respect to the pelvis using the planned implant sizes and positions. The separation between the implants, the separation between fixed points on the bones and the separation between a bone and an implant can be calculated. Any collisions can be looked for by varying the positions of the bones through a number of degrees of freedom, including flexion, abduction, adduction, extension, extrotation, introtation and introtFlexion. After a virtual range of motion analysis of the planned joint has been carried out, then at step 808 the plan can be saved if surgery is not immediately going to follow the planning procedure. In another embodiment, if surgery is to be carried out immediately, then the plan need not be saved and surgery can proceed.

FIG. 43 shows a flowchart illustrating a computer aided surgical method 820 for carrying out the hip implantation. The method begins at step 822 with the surgeon instructing the tracking control system to begin the image guided surgery operation. Then at step 824, using the surgical site display device the surgeon carries out a navigated single incision at the hip of the patient so as to provide access to the surgical site. Use of a single incision helps to provide a minimally invasive method. At step 826, the hip is distracted or otherwise separated in order to provide the surgeon with the required access to the surgical site. If the auto-registration procedure has previously been used, then the body part images are already registered. Alternatively, an intra-operative registration can be used similar to that described with reference to FIG. 37.

Irrespective of how registration is carried out, at step 828, a reamer or drilling device bearing a marker trackable by the tracking system is used to drill the acetabulum in a navigated manner so as to provide a cavity for receiving the acetabular cup implant at the planned position. At step 830, a trackable trail impactor tool is used to place a trail cup in the acetabular cavity in order to check the actual position of the cup relative to the planned position. If it is determined that the acetabular cavity is suitable, then at step 832, a trackable impactor tool is used to position the acetabular cup implant in the acetabulum and to position and orient the cup in accordance with the planned position which is graphically displayed as part of a navigated cup positioning procedure. The position and orientation of the implanted cup is detected and used to display an indication of the position and orientation of the cup so that the implanted position and orientation of the cup can be compared with the planned position and orientation and its position verified.

At step 834, a guide bearing a marker is attached to the femur to allow a navigated neck resection of the femur to be carried out at step 834. At step 836 reaming of a cavity in the femur is begun and at step 838, a broach with a marker in its handle is used to broach the cavity in the femur in a navigated manner. After the cavity has been completed, at step 840, a stem inserter tool bearing a marker is used to implant the femoral component within the femoral cavity and impact the femoral component into position. The position and orientation of the stem component is displayed and in particular the varus/valgus position, the anterior/posterior tilt, the anteversion, the depth and any deviation from the planned axis of the implant in the femur. At step 842, the hip resulting from the actual positions of the implants can be checked and the surgical plan can be updated using the detected positions of the implants to verify that the leg length and offset requirements have been met.

Then at step 844, an immediate assessment of the performance of the hip can be carried out. The alignment of the implanted orthopaedic implants can be displayed and the influence of the positions of the implants on the leg length, the offset and the range of motion can be displayed to the surgeon. Immediate post-operative assessment of the orthopaedic performance of the patient can be carried out by articulating the limbs and hip joint and observing a graphical representation of the position of the bones and/or implant components. Also the movement of the bones and/or implant components can be compared with a theoretical or model performance, with a pre-operative performance or assessed based on the surgeon's skill and experience. The surgical procedure then ends at step 846.

With reference to FIGS. 44A and 44B, there are shown side and transverse cross-sectional views of a further embodiment of an acetabular cup implant component 850. Although cup 850 as illustrated does not include a trackable marker, it can still be used in a navigated surgical procedure by implanting it using a marked impactor tool, which itself is navigated, as the position and orientation of the cup relative to the impact tool can have a fixed known relationship. In alternate embodiments a marker is provided at the apex of cup 850 in a manner similar to that described above with reference to FIGS. 40 and 41.

Acetabular cup 850 is particularly suited for use in an orthopaedic procedure in which only the articulate surfaces of the hip are replaced. The outer surface 852 of the cup is roughened to facilitate bone in growth. A preferred outer coating for the acetabular cup is that provided under the trade name Porocoat by DePuy International Ltd of the UK. The inner surface 854 of the acetabular cup, which provides the articulate surface of the hip joint, is highly polished. The cup 850 is made of a suitable bio-compatible material, such as a metal or alloy. In one embodiment, the cup is made of a cobalt chrome alloy. FIG. 44B is a cross-section along line AA of FIG. 44A.

FIG. 45A shows a perspective view of a femoral head implant 860. FIG. 45B shows a side elevation and FIG. 45C shows a transverse cross-section along line AA of FIG. 45B. Femoral head implant 860 can be used to replace the articulate surface of the femoral head. Implant 860 has a highly polished outer surface 861 in the general shape of a part of a sphere. Implant 860 has a stem or positioning pin 862 extending along an axis passing through the centre 864 of the sphere defined by the surface 861. A substantially annular cavity is defined by the wall of the implant and extends around the stem 862. The femoral head implant 860 can be made of a single unitary piece of material. The implant can be made of any suitable bio-compatible material, such as a metal or alloy. In one embodiment, the femoral head implant is made of a cobalt chrome alloy. Implant 860 can either be implanted using a navigable tool or can include a marker detectable by the tracking system, e.g. in stem 864 or within the wall of the implant.

FIG. 46A shows a perspective view of a further embodiment of a prosthetic femoral head implant 870. FIG. 46B shows a cross-section through femoral head implant 870. Implant 870 has the general shape of a part of a sphere and has a highly polished outer surface 871. A substantially annular cavity 872 extends around a longitudinal axis of the prosthetic head implant between an outer wall and an inner annual wall part 874 of the femoral head implant 870. Inner wall 874 defines a slightly tapering cavity 876 therein with a circular cross-section. Implant 870 can either be implanted using a navigable tool or can include a marker detectable by the tracking system, e.g. within the wall of the implant.

In use, prosthetic femoral head 870 can be used to replace the articulate surface of a femur. Prosthetic head implant 870 can be made of any suitable bio-compatible material, such as a metal or alloy. In one embodiment, it is made of a cobalt chrome alloy.

Images of the implants 850, 860, 880 and details of their geometry, and the same for any associated implanting tools or instrument, are provided in the planning and IGS software so that the positions of the implants can be planned and so that they can be implanted using an IGS procedure.

With reference to FIG. 47, there is shown a flowchart illustrating a computer aided method 880 for implanting prosthetic head implant 860. A number of method steps proceed and follow the described method steps as have already been described above.

Method 880 relates to the navigated surgical steps carried out by the surgeon. A virtual model of the implant 860 is used during planning the position of the implant.

In use, implant 860 is attached to the femoral neck via stem locating pin 862. At step 882, a trackable guide is positioned on the femoral head with a guide drilling axis coincidental with an axis of the femoral head/neck along which the implant stem 862 is eventually to lie. After the guide has been positioned and fixed to the femoral head, at step 884, a pilot hole can be drilled using the guide. In an alternate embodiment, a hole for receiving the stem 862 can be drilled at step 884. At step 886, using the pilot hole in the femoral head, the femoral head is resected into a shape to engage in cavity 863 in the implant. An image of a desired resected head shape can be displayed to the surgeon to guide the surgeon during this step. At step 888, if not already done so, then a hole for receiving the stem 862 is drilled using a navigated instrument to ensure that the hole is drilled along the correct axis and to the correct depth.

Then at step 890, the head implant, or a trial head, can be attached to the resected femoral head. The position of the implant can be compared with a planned position and when it is determined that the position is acceptable, then the head implant can be cemented in place. Alternatively, a trial head can be used prior to attaching the actual implant head 860 to check the actual position of the head compared to the planned position.

With reference to FIG. 48, there is shown a flowchart illustrating a computer aided surgical method 892 for implanting prosthetic femoral head implant 870 as shown in FIG. 46A. At step 894, the guide is attached to the femoral head at a planned position defined by the planning program. Then at step 896, the femoral head, and neck, if required, are resected to provide a tapered femoral neck section to engage within cavity 876. A trial implant can then be attached to the resected neck and a visual display of the actual position of the implant compared to the planned position of the implant can be displayed to the surgeon. If the actual position is acceptable, then at step 898, the prosthetic head 870 can be attached to the stem using a trackable instrument and the prosthetic head can be fixed to the femoral stem.

With reference to FIG. 49, there is shown a dummy part or virtual part of a human body 900 for use in training and teaching surgical procedures. The dummy is particularly suitable for use within the orthopaedic operating room. The dummy body includes an outer layer made of a material which mimics the behaviour of human skin. Outer skin layer 902 can be made of a polyurethane elastomer. Within the dummy body there are provided a number of dummy or synthetic bones made of a material which mimics an actual human bone. For example a synthetic femur 904 is provided as well as a pelvis, tibia and fibula, and parts of the ankle and knee joint.

Regions within the outer skin, not corresponding to joint regions are filled with a volume of material mimicking the performance of soft body tissue, e.g. volume 906 surrounding the femur. In a region surrounding a joint, e.g. the knee joint and the hip joint, a material which differs to the soft tissue material is used to mimic the behaviour and performance of a human joint. A volume of material is provided around and enclosing the joint. For example volume 908 surrounds the knee joint. A suitable material is a polyurethane elastomer. A further volume of joint material 910 is provided around the hip joint.

A synthetic or dummy ankle part 912 is also provided attached to the end of a synthetic tibia and/or fibula and enclosed within a volume of soft body tissue mimicking material. The dummy ankle part 912 can be made of a two part polyurethane resin. The dummy bones can be made of a solid foam which mimics the properties of dense cancellous bone. A suitable material is a solid foam, such as that provided by Synbone. A suitable material for the soft tissue mimicking material would be a two part expanding foam. A suitable polyurethane elastomer for the skin and joint enclosing parts would be the polyurethane elastomer provided under the trade name Smooth-On. A suitable two part polyurethane resin is that provided under the trade name Fast-Cast.

The particular materials used to provide the dummy body part 900 have been found to provide a particularly realistic dummy on which the orthopaedic procedures described herein, and other orthopaedic surgical procedures can be practised by a surgeon.

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

FIG. 50 illustrates a typical computer system that, when appropriately configured or designed, can serve as an image analysis apparatus of this invention. The computer system 1000 includes any number of processors 1002 (also referred to as central processing units, or CPUs) that are coupled to storage devices including primary storage 1006 (typically a random access memory, or RAM), primary storage 1004 (typically a read only memory, or ROM). CPU 1002 may be of various types including microcontrollers and microprocessors such as programmable devices (e.g., CPLDs and FPGAs) and unprogrammable devices such as gate array ASICs or general purpose microprocessors. As is well known in the art, primary storage 1004 acts to transfer data and instructions uni-directionally to the CPU and primary storage 1006 is used typically to transfer data and instructions in a bi-directional manner. Both of these primary storage devices may include any suitable computer-readable media such as those described above. A mass storage device 1008 is also coupled bi-directionally to CPU 1002 and provides additional data storage capacity and may include any of the computer-readable media described above. Mass storage device 1008 may be used to store programs, data and the like and is typically a secondary storage medium such as a hard disk. It will be appreciated that the information retained within the mass storage device 1008, may, in appropriate cases, be incorporated in standard fashion as part of primary storage 1006 as virtual memory. A specific mass storage device such as a CD-ROM 1014 may also pass data uni-directionally to the CPU.

CPU 1002 is also coupled to an interface 1010 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1002 optionally may be coupled to an external device such as a database or a computer or telecommunications network using an external connection as shown generally at 1012. With such a connection, it is contemplated that the CPU might receive information from the network, or might output information to the network in the course of performing the method steps described herein.

Although the above has generally described the present invention according to specific processes and apparatus, the present invention has a much broader range of applicability. In particular, aspects of the present invention is not limited to any particular kind of orthopaedic procedure and can be applied to virtually any joint or body structure. One of ordinary skill in the art would recognize other variants, modifications and alternatives in light of the foregoing discussion.

It will also be appreciated that the invention is not limited to the specific combinations of structural features, data processing operations, data structures or sequences of method steps described and that, unless the context requires otherwise, the foregoing can be altered, varied and modified. For example different combinations of structural features can be used and features described with reference to one embodiment can be combined with other features described with reference to other embodiments. Similarly the sequence of the methods step can be altered and various actions can be combined into a single method step and some methods steps can be carried out as a plurality of individual steps. Also some of the structures are schematically illustrated separately, or as comprising particular combinations of features, for the sake of clarity of explanation only and various of the structures can be combined or integrated together or different features assigned to other structures.

It will be appreciated that the specific embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description.

1. An integrated surgical system for use in an orthopaedic: operating room to enable a surgeon to carry out a computer aided surgical procedure on a subject, the integrated surgical system comprising:
a subject support on which the subject can be positioned;
a first wireless magnetic tracking system, the tracking system generating a magnetic field defining a working volume of the tracking system, the subject support being located at least partially within the working volume, and the tracking system including a tracking control system configured to track the position of a marker detectable by the tracking system within the working volume and generate a signal indicative of the position of the marker within a reference frame of the tracking system;
a registration system configured to register the position of the body part of the subject with an image of the body part of the subject within the reference frame of the tracking system;
at least a first display device configured to display a registered image of the body part of the subject and at least an image representative of a trackable implant during the computer aided surgical procedure;
a control system configured to integrate the functionalities of a plurality of the parts of the surgical system; and
a surgeon interface operable by the surgeon to control operation of the plurality of parts of the integrated surgical system.

2. The system of claim 1, further comprising a second wireless tracking system, the second wireless tracking system being an infrared wireless tracking system and being in communication with the control system and configured to generate a signal indicative of the position of a tracked element in the reference frame of the second wireless tracking system.

3. The system claim 1, wherein the first display device is a touch sensitive display and comprises a part of the surgeon interface.

4. The system of claim 1, wherein the surgeon interface includes an orientation sensitive device operable by a surgeon to enter control commands.

5. The system of claim 1, wherein the surgeon interface includes heads up display wearable by the surgeon and configured to display at least a one of the images selected from the group comprising: a captured image of the body part; an image of a model of the body part; a registered image of the body part; a video image of the body part; a representation of an implant; a representation of an instrument; an indication of the planned position of an implant, instrument or incision; and any combination of the preceding.

6. The system of claim 1, further comprising a wall display unit, the wall display unit being configured to provide a plurality of image regions, each image region being capable of displaying a different image.

7. The system of claim 6, wherein the different images are selected from the group comprising: a captured image of the body part; an image of a model of the body part; a registered image of the body part; a video image of the body part; a representation of an implant; a representation of an instrument; an indication of the planned position of an implant, instrument or incision; and any combination of the preceding.

8. The system of claim 1, device, the surgical site display device including an image display portion and a support, and wherein the image display portion is positionable over the surgical site of the patient in use.

9. The system of claim 8, wherein the surgical site display device includes an image capturing device having a field of view including the surgical site and generating a surgical site image, and wherein the surgical site image is displayed in the image display portion.

10. The system of claim 9, wherein the surgical site image is a real time video image of the surgical site.

11. The system of claim 9, wherein a further image is overlayed on the surgical site image and the further image and the surgical site image are displayed in the image display portion at the same time, and wherein the further image is selected from the group comprising: a captured image of the body part; an image of a model of the body part; a registered image of the body part; a video image of the body part; a representation of an implant; a representation of an instrument; an indication of the planned position of an implant, instrument or incision; and any combination of the preceding.

12. The system and further comprising an image capturing device which captures real time video images, and wherein the real time video images are displayed in real time on at least one display device of the system.

13. The system of claim 12, further comprising a surgical light, the surgical light being suspended and being movable to different positions and orientations with respect to the operating table, and wherein the image capturing device is provided as a part of the surgical light.

14. The system of claim 1, further comprising an image storage device storing a plurality of captured images of the body part of the subject, the images of the body part being selected from the group comprising: X-ray images; CT scan images; and X-ray fluoro images.

15. The system of claim 1, further comprising a body part model storage device, storing a plurality of generic 3-d models of different body parts.

16. The system of claim 1, further comprising an implant image storage device storing 3d images of a plurality of implants useable in the computer aided surgical procedure.

17. The system of claim 1, further comprising an instrument image storage device storing 3d images of a plurality of instruments useable in the computer aided surgical procedure.

18. The system of claim 1, wherein the registration system is an X-ray or X-ray fluoroscopy registration system.

19. The system of claim 18, wherein the registration system is configured to capture at least a first image and a second image of the body part from different directions with the patient on the operating table.

20. The system of claim 19, wherein the registration system includes a first x-ray source and a second x-ray source, a first detector positioned to capture the first image of the body part resulting from the first x-ray source and a second detector positioned to capture the second image of the body part resulting from the second x-ray source.

21. The system of claim 20, wherein the first detector and the second detector are positioned above the subject support and the first x-ray source and the second x-ray source are positioned below the subject support.

22. The system of claim 19, wherein the control system includes computer program instruction executable:
to generate a 3d image of the body part from the first image and second image;
to determine the position of the body part in the reference frame of the tracking system; and
to register the 3d image of the body part with the position of the body part in the reference frame of the tracking system.

23. The system of claim 1, wherein the tracking system includes a magnetic field generating subsystem and wherein the position of the magnetic field generating subsystem or subject support is movable so as to change the position or orientation of the working volume relative to the subject support.

24. The system claim 23, wherein a part of the subject support is movable and/or a part of the magnetic field generating subsystem is movable.

25. The system claim 21, wherein the first x-ray source and the second x-ray source are provided in a floor on which the subject support is located.

26. The system of claim 1, a video mixing and control subsystem which controls the display of images on a plurality of different image display parts of the system.

27. The system of claim 1, wherein the control system includes computer program instructions providing an orthopaedic surgery workflow program.

28. The system of claim 27, wherein the control system includes computer program instructions providing an orthopaedic implantation planning program.

29. The system of claim 28, wherein the control system includes computer program instructions providing an orthopaedic image guided surgery program for implementing the orthopaedic procedure at least partially planned by the orthopaedic planning program.

30. The system claim 27, wherein the tracking system passes data indicating the identity of a marker being tracked by the tracking system to the control system, and wherein the control system determines whether the marker is associated with the position of a bone, an implant or an instrument.

31. The system of claim 1, further comprising at least one marker wirelessly trackable by the tracking system.

32. The system of claim 31, wherein the marker is attached to an implant.

33. The system of claim 31, wherein the marker is attached to an instrument.

34. The system of claim 31, wherein the marker has a housing including a bone anchor for retaining the marker within the bone of the subject and wherein the marker is hermetically sealed in the housing.

35. The system of claim 34, wherein the housing is configured to be percutaneously implantable within the bone of a subject.

36. The system of claim 1, further comprising a prosthetic joint, the prosthetic joint comprising a first orthopaedic implant bearing a first marker wirelessly trackable by the tracking system and a second orthopaedic implant bearing a second marker wirelessly trackable by the tracking system.

37. The system of claim 36, wherein the prosthetic joint is a prosthetic knee joint, the first orthopaedic implant is a femoral component and the second orthopaedic component is a tibial component, and the femoral component includes a locating pin which in use is located within the femur and the first marker is located at least partially within the locating pin, and the tibial component includes a keel which in use is located within the tibia and the second marker is located at least partially within the keel.

38. The system of claim 36, wherein the prosthetic joint is a prosthetic hip joint, the first orthopaedic implant is an acetabular component and the second orthopaedic component is a femoral component, and the acetabular component is a cup and the first marker is located within a wall of the cup at an apex of the cup, and the femoral component includes a body and the second marker is located at least partially within the body.

39. The system of claim 1, wherein the system includes at least three markers wirelessly trackable by the wireless magnetic tracking system, and wherein:
a first of the three markers is configured to be powered by RF induction and is implantable in the bone of the subject;
a second of the three markers is configured to be powered by RF induction and is attachable to an orthopaedic implant for implanting in the body of the subject; and
a third of the three markers has a battery and is attachable to an instrument for use in the surgical procedure of implanting the orthopaedic implant in the body of the subject.

40-95. (canceled)
